(19) United States
(12) Patent Application Publication
(10) Pub. No.: US 2023/0054039 A1
(43) Pub. Date: Feb. 23, 2023

(54) METHOD OF TREATING HER2-POSITIVE BREAST CANCER

(71) Applicant: University of Cincinnati, Cincinnati, OH (US)

(72) Inventors: Jun-Lin Guan, Cincinnati, OH (US); Mingang Hao, Cincinnati, OH (US)

(21) Appl. No.: 17/815,849

(22) Filed: Jul. 28, 2022

Related U.S. Application Data
(60) Provisional application No. 63/226,210, filed on Jul. 28, 2021.

(51) Int. Cl. A61K 31/7105; A61P 35/02; A61K 45/06; A61K 38/46

(52) U.S. Cl. CPC A61K 31/7105; A61P 35/02; A61K 45/06; A61K 38/465

(57) ABSTRACT

A method of treating human epidermal growth factor receptor 2 (HER2)-positive breast cancer in a subject in need thereof is provided, including administering to the subject an effective amount of a therapeutic agent that inhibits a nucleic acid that encodes FAK family-interacting protein of 200 kDa (FIP200). Also provided is a method of inhibiting metastasis of human epidermal growth factor receptor 2 (HER2)-positive breast cancer in a subject.

### CROSS-REFERENCE TO RELATED APPLICATIONS

This application claims priority to U.S. Provisional Application Ser. No. 63/226,210, filed Jul. 28, 2021, the entire contents of which are incorporated herein by reference.

### STATEMENT REGARDING FEDERALLY-SPONSORED RESEARCH OR DEVELOPMENT

This invention was made with government support under CA211066, awarded by the National Institutes of Health. The government has certain rights in the invention.

### TECHNICAL FIELD

The present disclosure relates to the field of cancer therapy. Specifically, the disclosure relates to methods of treating human epidermal growth factor receptor 2 (HER2)-positive breast cancer by inhibiting FAK family-interacting protein of 200 kDa (FIP200)-mediated autophagy.

### SEQUENCE LISTING

A Sequence Listing XML having file name Sequence_Listing_CIN0371PA.xml, created on Jul. 27, 2022 (109,000 bytes), is incorporated herein by reference. The nucleic and amino acid sequences listed in the accompanying sequence listing are shown using standard abbreviations as defined in 37 C.F.R. 1.822.

### BACKGROUND

Breast cancer is the most common malignancy for women in the United States and remains a major health threat with high incidence and lethality. It is a heterogeneous disease with different subtypes characterized by various driver mutations, transcriptome profiles, metastatic potentials, and responses to different treatments. HER2 is a member of the ErbB tyrosine kinase receptor family. Amplification of HER2 is a driver mutation for approximately 25% of human breast cancers, which have high metastasis incidence and poor prognosis. Targeted therapy with anti-HER2 monoclonal antibodies or dual EGF receptor/HER2 tyrosine kinase inhibitors have significantly improved outcomes for patients of HER2-enriched breast cancer. Nevertheless, resistance to these HER2-targeted therapies in some patients and relapse for others remain a significant challenge for effective treatment for the disease.

Autophagy is an evolutionarily conserved catabolic process that mediates the clearance of cytosolic proteins and organelles for maintaining cellular homeostasis. Defective autophagy has been implicated in multiple human diseases including neurodegeneration and cancer. Due to its impact on many cellular functions of both tumor cells and stromal cells in the tumor microenvironment, autophagy has been shown to play both tumor suppressive and tumor promoting functions in different contexts and models. Interestingly, compared to other subtypes of breast cancer, HER2-enriched breast cancer displays a lower autophagy gene signature. Indeed, previous studies have shown that HER2 can suppress autophagic flux by physically tethering and dissociating Beclinl from the Vps34-Vps15 complex. Another study reported that increasing autophagic flux by genetically disrupting interaction of Beclin 1 with Bcl-2 inhibited mammary tumorigenesis in the MMTV-Neu mouse model of breast cancer (Vega-Rubin-de-Celis, et al., Increased autophagy blocks HER2-mediated breast tumorigenesis, Proc. Nat'l Acad. Sci. USA 115(16): 4176-81 (2018)), supporting a tumor suppressive function of autophagy in HER2-enriched breast cancer. However, these results were in contrast to the tumor promoting functions of autophagy shown in many earlier studies (Poillet-Perez, et al., Autophagy maintains tumour growth through circulating arginine, Nature 563(7732): 569-737 (2018)); Strohecker, et al., Autophagy sustains mitochondrial glutamine metabolism and growth of BrafV600E-driven lung tumors, Cancer Discovery 3(11): 1272-85 (2013)).

Deletion of an essential autophagy gene, Fip200 (FAK-family Interacting Protein of 200 kDa; also called RB1CC1), has been shown to decrease mammary tumor development, growth, and metastasis driven by polyomavirus middle T-antigen (PyMT) oncoprotein. Wei, et al., Suppression of autophagy by FIP200 deletion inhibits mammary tumorigenesis, Genes Dev. 25(14): 1510-27 (2011). This study provided evidence for a pro-tumorigenesis role for autophagy in animals with intact immune systems. While it is possible that autophagy may play opposing roles in different subtypes of breast cancer driven by various driver mutations, an alternative and emerging hypothesis is that some of the effects of inactivation of various autophagy genes are due to the loss of their non-autophagy functions. For example, besides its well-characterized role in autophagy, FIP200 has been shown to interact with other proteins to regulate diverse cellular functions (Gan and Guan, FIP200, a key signaling node to coordinately regulate various cellular processes, Cell Signal. 20(5): 787-94 (2008)). Likewise, Beclinl has been shown to control p53 levels through regulation of the deubiquitination activity of USP10 and USP13, which may contribute to its tumor suppressive function independent of or in addition to its role in autophagy (Liu, et al., Beclin1 controls the levels of p53 by regulating the deubiquitization activity of USP10 and USP13, Cell 147(1): 223-34 (2011)). Moreover, disrupting the Beclinl interaction with Bcl-2 may affect tumor development through other mechanisms, given the multiple and paradoxical roles of Bcl-2 in cancer (Adams and Cory, Bcl-2-regulated apoptosis: mechanism and therapeutic potential, Curr. Opin. Immunol. 19(5): 488-96 (2007)). Indeed, other previous in vitro studies suggest that autophagy can facilitate the development of resistance of HER2-positive breast cancer cells to anti-HER2 antibody, although such a tumor promoting role has not been examined in vivo.

Small extracellular vesicles (sEVs) are a group of extracellular vesicles ranging in size of 40-150 nm that are generated in multi-vesicular bodies (MVBs) and released from various cells. Research studies indicate an increasing importance of sEVs as an essential mechanism for the communication of tumor cells with both surrounding stromal cells and distal organs. sEVs are now understood to have widespread influence on tumor growth, invasion and metastasis, and drug resistance. sEVs are initially formed as intraluminal vesicles (ILVs) by inward budding of the limiting membrane of MVBs. Upon fusion of MVBs with the plasma membrane, ILVs are released from MVBs as sEVs that can enter other cells to regulate various cellular functions. Recent studies suggest regulation of sEVs, including their biogenesis and secretion, by a number of autophagy genes through both autophagy and non-canonical autophagy functions in different cells. However, the role and mechanisms of sEVs in mediating autophagy in the regulation of tumorigenesis and progression have not been investigated in mouse models of breast or other cancers in vivo.

A need exists for improved methods of inhibiting tumorigenesis and metastasis of HER2-positive breast cancer.

### SUMMARY

It has now been found that inhibition of FIP200-mediated autophagy blocks tumorigenesis of HER2-positive breast cancer by diverting HER2 from the surface of breast cancer cells to sEVs, which are then released from the breast cancer cells. Ablating FIP200-mediated autophagy has been shown to arrest tumor growth and metastasis in a mouse model of HER2-positive breast cancer. Accordingly, provided herein are methods of treating HER2-positive breast cancer by inhibiting FIP200-mediated autophagy.

In one embodiment, a method of treating human epidermal growth factor receptor 2 (HER2)-positive breast cancer in a subject in need thereof is provided, the method comprising administering to the subject an effective amount of a therapeutic agent that inhibits a nucleic acid that encodes FAK family-interacting protein of 200 kDa (FIP200).

In another embodiment, a method of inhibiting metastasis of human epidermal growth factor receptor 2 (HER2)-positive breast cancer in a subject in need thereof is provided, the method comprising administering to the subject an effective amount of a therapeutic agent that inhibits FAK family-interacting protein of 200 kDa (FIP200)-mediated autophagy.

These and other objects, features, embodiments, and advantages will become apparent to those of ordinary skill in the art from a reading of the following detailed description and the appended claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A. Kaplan-Meier tumor-free survival plot of Ctrl (n=32), cKO (n=34) and cKI (n=20) mice. Log-rank (Mantel-Cox) test, P<0.0001.

FIG. 1B. Tumor lysates from Ctrl and cKO mice were analyzed by immunoblotting for Fip200, p62 and GAPDH.

FIG. 1C. Representative whole-mount staining of the inguinal mammary glands from Ctrl, cKO and cKI mice at 30 weeks of age. Tumor lysates from Ctrl and cKO mice were analyzed by immunoblotting for Fip200, p62 and GAPDH. Scale bar: 500 μm. n=3.

FIG. 1D. Representative hematoxylin and eosin (H&E) staining of the mammary glands of 30-weeks old Ctrl, cKO and cKI mice. Scale bars: 200 μm, 50 μm for insets. n=3.

FIG. 1E. Mammary gland sections of Ctrl, cKO and cKI mice at 30 weeks were immunostained with antibodies against Ki-67 or p62. Quantification of the mean value for each immunoreactivity score (IRS) is shown at the right panel. Error bars indicate mean±SEM. n=3 replicates. **P<0.01, 2-tailed Student's t test. Scale bar: 50 μm.

FIG. 1F. Mammary gland sections of Ctrl, cKO and cKI mice at 30 weeks or 50 weeks of age were immunostained with antibodies against HER2.

FIG. 1G. Mammary gland sections of Ctrl and cKO mice at 63 weeks of ages were immunostained with antibodies against HER2, p62, and p-Akt (S473).

FIG. 2A. Schematics for the preparation of Fip200+/+ and Fip200−/− mammary tumor cells from tumors developed in Ctrl (i.e., MMTV-Neu; fip200f/f) mice.

FIG. 2B. Immuno-blots showing levels of Fip200, HER2 and GAPDH in Fip200+/+ and Fip200−/− cells (prepared from mammary tumors in two different mice N148 and N418). 2C.

FIG. 2C. Kaplan-Meier tumor-free survival plot in FVB mice transplanted with Fip200+/+ and Fip200−/− cells. n=5.

FIG. 2D. Tumor growth curves for indicated genotypes of N418 cells. n=5 mice. *P<0.05, **P<0.01, 2-tailed Student's t test.

FIG. 2E. Tumor weight at endpoint for indicated genotypes. n=5 mice. **P<0.01, 2-tailed Student's t test.

FIG. 2F. Immuno-blots showing levels of HER2, Atg5 and GAPDH in mammary tumor cells from MMTV-Neu; fip200f/f mouse N148 with or without Atg5 shRNA knockdown.

FIG. 2G. Immuno-blots showing levels of HER2, Atg5 and GAPDH in mammary tumor cells from MMTV-Neu; fip200f/f mouse N418 with or without Atg5 shRNA knockdown.

FIG. 2H. Immuno-blots showing levels of HER2, FIP200, ATG13 and GAPDH in WT, FIP200 KO and Atg13 KO HeLa cells.

FIG. 2I. Immuno-blots showing levels of HER2 and GAPDH in HeLa cells treated by HBSS starvation, chloroquine (50 μM), bafilomycin (200 nM), 3-methyladenine (3-MA) (5 mM) and spautin-1 (10 μM) for 48 hrs.

FIG. 2J. Immuno-blots showing levels of HER2, FIP200 and GAPDH in WT and FIP200 KO MEFs.

FIG. 3A. Immuno-blots showing levels of HER2 and GAPDH in Fip200+/+ (upper panels, short exposure) and Fip200−/− (lower panels, long exposure) cells after incubation with cycloheximide (CHX; 25 mg/ml) for different times as indicated.

FIG. 3B. Quantification of immunoblots in FIG. 3A (error bars indicate mean±SEM). n=3 replicates **P<0.01, 2-tailed Student's t test.

FIG. 3C. Immunoblots showing levels of HER2, ubiquitin and GAPDH in Fip200+/+ and Fip200−/− cells treated with DMSO and MG132 (10 μM) for 6 hours.

FIG. 3D. Immunoblots showing levels of HER2, ubiquitin and GAPDH in WT, FIP200 KO and Atg13 KO HeLa cells treated with DMSO and MG132 (10 μM) for 6 hours.

FIG. 3E. Mammary glands sections of 30-week-old Ctrl, cKO and cKI mice were analyzed by immunofluorescent staining using anti-HER2 antibody (top panels) and DAPI staining (bottom panels). Scale bar: 20 μm. Graph at right panel shows quantification of fluorescence density of plasma membrane versus cytoplastic HER2, n=3 replicates, **P<0.01, ***P<0.001, 2-tailed Student's t test.

FIG. 3F. Flow cytometry analysis of mammary tumor cells from 63-week-old Ctrl and cKO mice using antibodies for HER2, EpiCAM and Cd24, as indicated. Error bars indicate mean±SEM. n=3 replicates. *P<0.05.

FIG. 3G. Fip200+/+ and Fip200−/− cells from two independent mice (N148 and N418) were analyzed by immunofluorescent staining using anti-HER2 antibody and DAPI staining. Scale bar: 10 μm. Graph at right panel shows quantification of fluorescence density of plasma membrane versus cytoplastic HER2, n=3 replicates, **P<0.01, ***P<0.001, 2-tailed Student's t test.

FIG. 3H. WT, FIP200 KO and ATG13 KO HeLa cells were analyzed by immunofluorescent staining using anti-HER2 antibody (top panels) and DAPI staining (bottom panels). Scale bar: 20 μm. Graph at bottom panel shows quantification of fluorescence density of plasma membrane versus cytoplastic HER2, n=3 replicates, **P<0.01, ***P<0.001, 2-tailed Student's t test.

FIG. 4A. Fip200+/+ and Fip200−/− cells were analyzed by co-immunofluorescent staining using anti-HER2 and anti-Rab7 antibodies, and DAPI staining. Scale bars: 10 μm (left panels) and 2.5 μm (right panels). Graph shows quantification of co-localization of intracellular HER2 (green) and Rab7 (red) by Pearson's coefficients, n=3 replicates, ***P<0.001, 2-tailed Student's t test.

FIG. 4B. WT, FIP200 KO and ATG13 KO HeLa cells were co-transfected with expression vectors encoding GFP-HER2 and mCherry-Rab7 and examined under a fluorescent microscope. Scale bars: 10 μm. Graph shows quantification of co-localization of intracellular HER2-GFP and mCherry-Rab7 by Pearson's coefficients. n=3 replicates, ***P<0.001, 2-tailed Student's t test.

FIG. 4C. WT, FIP200 KO and ATG13 KO HeLa cells were co-transfected with expression vectors encoding GFP-HER2 mCherry-Rab5Q79L and examined under a fluorescent microscope. Scale bars: 5 Graph shows line-scan profile of fluorescence intensity of intracellular HER2-GFP and mCherry-Rab5Q79L (top right) and quantification of mean intensity of GFP within circular mCherry (bottom panels) n=3 replicates, ***P<0.001, 2-tailed Student's t test.

FIG. 4D. WT, FIP200 KO and ATG13 KO HeLa cells were transfected with an expression vector encoding GFP-HER2. Cells were stained by anti-Golgi-97 and DAPI. Scale bars: 5 μm; and for enlarged bottom panels: 2.5 Graph (far right) shows quantification of co-localization of intracellular HER2 (green) with Golgi-97 (red) or Turquoise2 (blue) by Pearson's coefficients, n=3 replicates, ***P<0.001, 2-tailed Student's t test.

FIG. 4E. WT, FIP200 KO and ATG13 KO HeLa cells were co-transfected with expression vectors encoding GFP-HER2 and Turquoise2-Golgi. Cells were examined directly by a fluorescent microscope. Scale bars: 5 Graph (bottom panel) shows quantification of co-localization of intracellular HER2 (green) with Golgi-97 (red) or Turquoise2 (blue) by Pearson's coefficients, n=3 replicates, ***P<0.001, 2-tailed Student's t test.

FIG. 5A. Mammary glands sections of 30-week-old Ctrl, cKO and cKI mice were analyzed by co-immunofluorescent staining using anti-HER2 and anti-CD81 antibodies and DAPI staining. Scale bar: 2 Graph (bottom panel) shows quantification of co-localization of intracellular HER2 with indicated markers by Pearson's coefficients, n=3 replicates, **P<0.01, ***P<0.001, 2-tailed Student's t test.

FIG. 5B. Fip200+/+ and Fip200−/− cells were analyzed by co-immunofluorescent staining using anti-HER2 and anti-CD63 antibodies, and DAPI staining. Scale bars: 5μm (left panels) and 2.5 μm (right panels). Graph (far right) shows quantification of co-localization of intracellular HER2 with indicated markers by Pearson's coefficients, n=3 replicates, **P<0.01, ***P<0.001, 2-tailed Student's t test.

FIG. 5C. WT, FIP200 KO and ATG13 KO HeLa cells were transfected with an expression vector encoding GFP-HER2, and then stained by anti-CD63 antibodies and DAPI, and examined under a fluorescent microscope. Scale bar: 10 Graph (far right) shows quantification of co-localization of intracellular HER2 with indicated markers by Pearson's coefficients, n=3 replicates, **P<0.01, ***P<0.001, 2-tailed Student's t test.

FIG. 5D. WT, FIP200 KO and ATG13 KO HeLa cells were co-transfected with an expression vector encoding GFP-HER2 and RFP-CD81 and then stained by DAPI, and examined under a fluorescent microscope. Scale bar: 10 Graph (far right) shows quantification of co-localization of intracellular HER2 with indicated markers by Pearson's coefficients, n=3 replicates, **P<0.01, ***P<0.001, 2-tailed Student's t test.

FIG. 6A. Immunoblot showing levels of HER2, Grp94 and Alix in the sEVs isolated from equal numbers of Fip200+/+ and Fip200−/− cells. n=5 replicates. *P<0.05.

FIG. 6B. Protein contents in sEVs isolated from equal numbers of Fip200+/+ and Fip200−/− cells. n=5 replicates. *P<0.05.

FIG. 6C. Immunoblot showing levels of HER2, Grp94 and Alix in the sEVs isolated from equal amounts of protein lysates from Fip200+/+ and Fip200−/− cells cells. n=5 replicates. *P<0.05.

FIG. 6D. Immunoblot showing levels of HER2, Grp94 and Alix in the sEVs isolated from equal numbers of WT, FIP200 KO and ATG13 KO HeLa cells. n=5 replicates. ***P<0.001.

FIG. 6E. Immunoblot showing levels of HER2, Grp94 and Alix in the sEVs isolated from equal number of sEVs. n=5 replicates. ***P<0.001.

FIG. 6F. Number of sEVs isolated from equal numbers of WT, FIP200 KO and ATG13 KO HeLa cells. n=5 replicates. ***P<0.001.

FIG. 6G. WT, FIP200 KO and ATG13 KO HeLa cells were co-transfected with expression vectors encoding GFP-CD63 and mCherry-Rab5Q79L, and examined under a fluorescent microscope. Scale bar: 5 Graphs (middle) show the line-scan profile of fluorescence intensity of intracellular CD63-GFP and mCherry-Rab5Q79L. Graph (far right) shows quantification of mean intensity of GFP within circular mCherry. n=3 replicates, **P<0.01, 2-tailed Student's t test.

FIG. 6H. Immunoblot showing levels of HER2, Rab27a and GAPDH in HeLa WT, FIP200 KO and ATG13 KO cells transfected with scramble control or siRNAs targeting Rab27a.

FIG. 6I. Illustration of models normal (left) or altered (right) intracellular trafficking of HER2 and its release from tumor cells upon autophagy blockade.

FIG. 7. Minimal levels of apoptosis in mammary glands of 30 week old mice. Mammary gland sections of 30-weeks-old Ctrl, cKO and cKI mice were immunostained by anti-Caspase-3. Scale bar: 50 μm.

FIG. 8A. Equal amounts of HER2 Fip200+/+ and Fip200−/− cells plated into 96-well plate were monitored by Incucyte every hour to compare the percentage of phase confluence. Error bars indicate mean±SEM. n=3 replicates. * denotes P<0.05.

FIG. 8B. Representative images (left, middle) and quantification (right) of migrated cells in trans-well migration assays for indicated cells. Error bars indicate mean±SEM. n=3 replicates. * denotes P<0.05. scale bar: 100

FIG. 9A. Quantitative-RT-PCR analysis of HER2 mRNA levels in total RNAs from Fip200+/+ and Fip200−/− cells. Error bars indicate mean±SEM. n=3 replicates. *P<0.05, ***P<0.001.

FIG. 9B. Quantitative-RT-PCR analysis of HER2 mRNA levels in total RNAs from Fip200+/+ cells treated with or without Atg5 shRNA knockdown. Error bars indicate mean±SEM. n=3 replicates. *P<0.05, ***P<0.001.

FIG. 9C. Quantitative-RT-PCR analysis of HER2 mRNA levels in total RNAs from WT, FIP200 KO and ATG13 KO HeLa cells (9C). Error bars indicate mean±SEM. n=3 replicates. *P<0.05, ***P<0.001.

FIG. 10A. Immuno-blot analysis of FIP200 and GAPDH in WT and FIP200 KO MCF-7 cells.

FIG. 10B. Representative immunofluorescent images of ectopic expression of GFP-HER2 fusion protein in living WT and FIP200 KO MCF-7 cells. Scale bar: 10 μm.

FIG. 11. Quantification of the number of intracellular HER2-GFP punta. WT, FIP200 KO and ATG13 KO HeLa cells were transfected with an expression vector encoding GFP-HER2 and examined under a fluorescent microscope. Scale bar: 10 Graph shows the number of intracellular HER2-GFP puncta per cell in each genotype, **P<0.01, 2-tailed Student's t test.

FIG. 12. Co-localization of HER2 and Rab5a in HeLa cells. Representative immunofluorescent micrographs of GFP-HER2 and RFP-Rab5 fusion protein in HeLa WT, FIP200 KO and ATG13 KO cells. Scale bar: 10 Graphs shows quantification of colocalization of intracellular HER2 and RFP-Rab5 by pearson's coefficients, n=3 replicates, **P<0.01, 2-tailed Student's t test.

FIG. 13A. Live WT, FIP200 KO and ATG13 KO HeLa cells were incubated with Alexa Fluor® 647 anti-HER2 at 37 ° C. for 3 hrs. They were examined at the beginning (top panels) or end (bottom panels) of the incubation by confocal microscopy. Scale bar: 10 μm.

FIG. 13B. Quantification of internalized HER2 as number of puncta per cell in bottom panels of A. Error bars indicate mean±SEM. n=11.

FIG. 13C. Immuno-blots showing levels of HER2 and GAPDH in WT, FIP200 KO and ATG13 KO HeLa cells with or without treatment of 20 μM Dynasore for 24 hours, as indicated.

FIG. 14A. Quantification of sEV number in the CMs from equal numbers of HER2 Fip200+/+ and Fip200−/− cells. Error bars indicate mean±SEM. n=5.

FIG. 14B. Nanoparticle tracking analysis (NTA) of sEVs derived from HER2 Fip200+/+ and Fip200−/− cells indicating the size of vesicles.

FIG. 14C. Quantification of sEVs diameter (nm) in FIG. 14B. Error bars indicate mean±SEM. n=5 replicates.

FIG. 15A. Nanoparticle tracking analysis (NTA) to measure size of sEVs derived from HeLa WT, FIP200 KO and ATG13 KO cells.

FIG. 15B. Quantification of sEVs diameter (nm) in FIG. 15A. Error bars indicate mean ±SEM. n=5 replicates.

FIG. 16A. ELISA assay showing the concentration of Neu protein in the sEVs from equal volume of serum of mice bearing Fip200+/+ or Fip200−/− orthotopic tumor. *P<0.05.

FIG. 16B. Representative images of the lung lobes of mice inoculated with Fip200+/+ and Fip200−/− tumor cells through tail vein injection.

FIG. 16C. Representative H&E staining of the lung lobes of mice inoculated with Fip200+/+ and Fip200−/− tumor cells through tail vein injection. Scale bar: 1 mm. *P<0.05.

### DETAILED DESCRIPTION

The following description of particular embodiment(s) is merely exemplary in nature and is in no way intended to limit the scope of the invention, its application, or uses, which may, of course, vary. The invention is described with relation to the non-limiting definitions and terminology included herein. These definitions and terminology are not designed to function as a limitation on the scope or practice of the invention but are presented for illustrative and descriptive purposes only.

The terminology used herein is for the purpose of describing particular embodiments only and is not intended to be limiting. As used herein, the singular forms “a,” “an,” and “the” are intended to include the plural forms, including “at least one,” unless the content clearly indicates otherwise. “Or” means “and/or.” As used herein, the term “and/or” includes any and all combinations of one or more of the associated listed items. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof. The term “or a combination thereof” means a combination including at least one of the foregoing elements.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, pH, size, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Unless otherwise defined, all terms (including technical and scientific terms) used herein have the same meaning as commonly understood by one of ordinary skill in the art to which this disclosure belongs. It will be further understood that terms such as those defined in commonly used dictionaries should be interpreted as having a meaning that is consistent with their meaning in the context of the relevant art and the present disclosure, and will not be interpreted in an idealized or overly formal sense unless expressly so defined herein. The term “subject,” as used herein, means any mammalian subject, including mice, rats, rabbits, pigs, monkeys, humans, and the like. In a specific embodiment, the subject is a human patient.

The terms “treat,” “treatment,” and “treating,” as used herein, refer to a method of alleviating or abrogating a disease, disorder, and/or symptoms thereof. In a specific embodiment, the disease or disorder is breast cancer. In a more specific embodiment, the disease to be treated is HER2-positive breast cancer.

An “effective amount,” as used herein, refers to an amount of a substance (e.g., a therapeutic compound and/or composition) that elicits a desired biological response. In some embodiments, an effective amount of a substance is an amount that is sufficient, when administered to a subject suffering from or susceptible to a disease, disorder, and/or condition, to treat, diagnose, prevent, and/or delay and/or alleviate one or more symptoms of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of a formulation to treat a disease, disorder, and/or condition is the amount that alleviates, ameliorates, relieves, inhibits, prevents, delays onset of; reduces severity of and/or reduces incidence of one or more symptoms or features of the disease, disorder, and/or condition. Furthermore, an effective amount may be administered via a single dose or via multiple doses within a treatment regimen. In some embodiments, individual doses or compositions are considered to contain an effective amount when they contain an amount effective as a dose in the context of a treatment regimen. Those of ordinary skill in the art will appreciate that a dose or amount may be considered to be effective if it is or has been demonstrated to show statistically significant effectiveness when administered to a population of patients; a particular result need not be achieved in a particular individual patient in order for an amount to be considered to be effective as described herein.

In the present disclosure, genetic approaches were used to delete the essential autophagy gene Fip200 as well as specifically block its autophagy functions with a newly generated mutant knock-in allele in an MMTV-Neu mouse model. Experiments were carried out to investigate the role and functions of autophagy in HER2-driven breast cancer. Results showed that autophagy blockade abolished mammary tumorigenesis by directly regulating oncogenic driver HER2 in this model. Autophagy inhibition, while not affecting HER2 mRNA levels or protein degradation through either proteasome or lysosomal pathways, altered intracellular trafficking of HER2 to the ILVs within MVBs and subsequent releases through sEVs. Thus, the present disclosure demonstrates a new mechanism of autophagy regulation mediated by sEVs to reduce the plasma membrane expression of HER2 as an oncogenic driver in the tumor cells, to decrease breast cancer development in vivo. The present disclosure provides a new therapeutic strategy for the treatment of HER2-positive breast cancer based on distinct mechanisms that differ from the current targeted therapies.

The present inventors hypothesized that at least part of the reasons for the conflicting outcomes in the current literature for the various studies by knockout of essential autophagy genes could be due to the loss of their non-canonical autophagy functions. Thus, a rigorous genetic approach was taken to compare the effects of deleting an essential autophagy gene (i.e. FIP200 cKO) with that of specifically blocking its autophagy functions (i.e. FIP200 cKI) in vivo. In the present disclosure, results show that both FIP200 cKO and cKI virtually abolished mammary tumorigenesis in the MMTV-Neu mouse model of breast cancer, providing compelling genetic evidence for a tumor promoting function of autophagy in HER2-positive breast cancer. Further, autophagy promotes HER2-driven tumorigenesis by maintaining HER2 localization on the plasma membrane; blocking autophagy diverted HER2 trafficking to MVBs, and then HER2 was released from tumor cells in sEVs, leading to decreased levels of HER2 on the cell surface for tumorigenesis. These results reveal a regulatory mechanism of autophagy in cancer cells by controlling the oncogenic driver HER2 directly, supporting autophagy inhibition as a distinct treatment strategy that may synergize with current anti-HER2 agents.

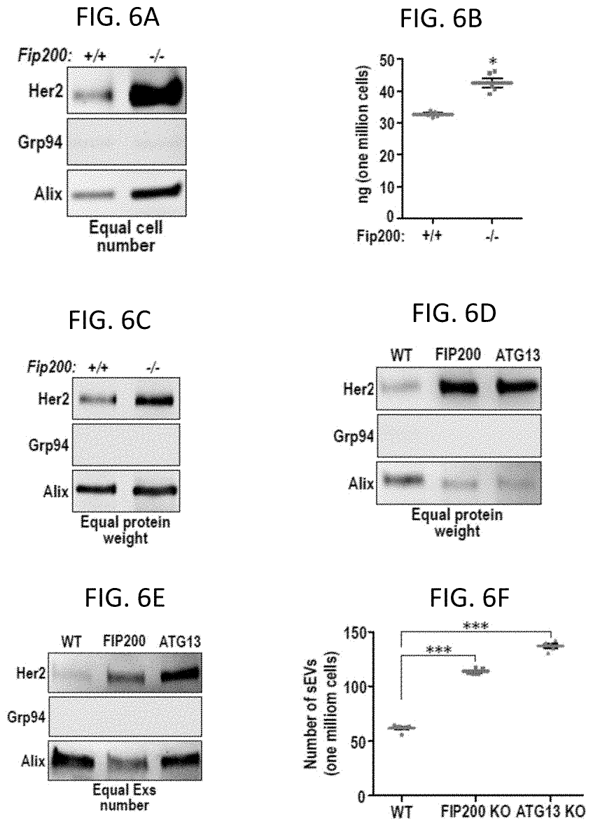

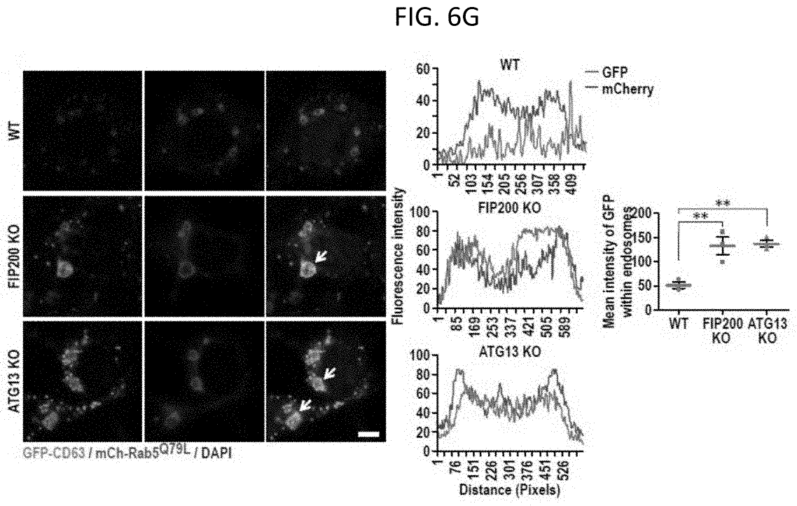

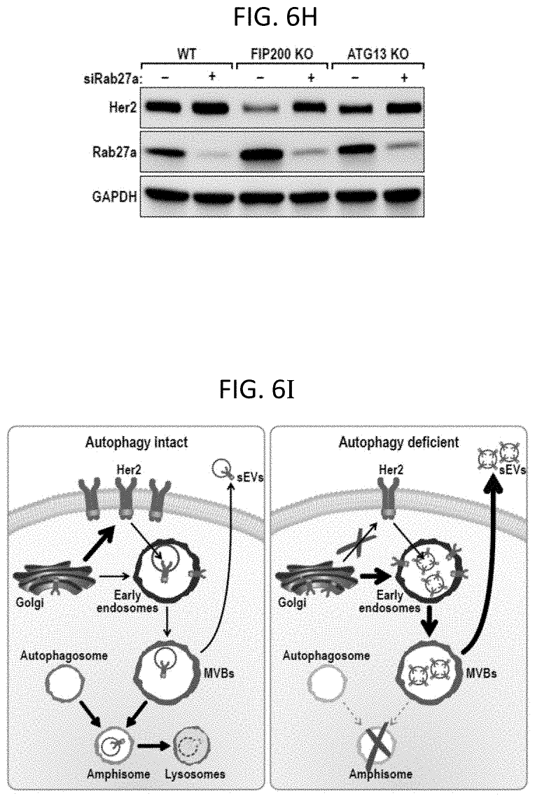

The present disclosure employed using both mouse models in vivo and multiple cell systems in vitro to elucidate a new role for sEVs to regulate tumor cell functions by releasing oncogenic driver HER2 from cells to inhibit their tumorigenic activity. This is distinct from the well-established classic function of sEVs to deliver various bioactive molecules to recipient cells in inter cellular communication for cancer development and progression. Results showed significantly increased HER2 association with both ILVs in MVBs in tumor cells and sEVs released from the cells, in correlation with the reduced levels on the plasma membrane, in autophagy-deficient cells. In addition to the increased incorporation of HER2 for the same amount of sEVs, an increased amount of sEVs upon autophagy inhibition was also observed. While not desiring to be bound by theory, it is believed that autophagy blockade diverts HER2 trafficking from the Golgi through endosomes for release from tumor cells in sEVs (FIG. 6I). The present disclosure demonstrates a tumor promoting function for autophagy in HER2-positive breast cancer model, providing further support for targeting autophagy in multiple subtypes of breast cancer. Importantly, FIP200 deletion exhibited a greater inhibitory effect by substantially blocking tumorigenesis in MMTV-Neu model compared to the other models, which is believed to be due to its direct effect on the cell surface expression of the oncogenic driver HER2.

As described herein, the increased association of HER2 with sEVs and release from tumor cells correlated with reduced HER2 expression on the tumor cell surface and reduced tumorigenicity in cKO mice. These results evidence a novel mechanism of HER2 trafficking diverted to sEVs to compromise HER2 oncogenic signaling in tumor cells upon autophagy inhibition to decrease tumorigenicity in vivo.

In one embodiment, a method is provided for treating human epidermal growth factor receptor 2 (HER2)-positive breast cancer in a subject in need thereof, the method comprising administering to the subject an effective amount of a therapeutic agent that inhibits a nucleic acid that encodes FAK family-interacting protein of 200 kDa (FIP200).

In another embodiment, a method is provided for inhibiting metastasis of human epidermal growth factor receptor 2 (HER2)-positive breast cancer in a subject in need thereof, the method comprising administering to the subject an effective amount of a therapeutic agent that inhibits FAK family-interacting protein of 200 kDa (FIP200)-mediated autophagy.

As used herein, “HER2-positive” or “HER2+” refers to a breast cancer tumor or breast cancer cell that has higher than normal HER2 protein levels. In embodiments, a HER2-positive breast cancer has a HER2 immunohistochemistry (IHC) score of 3+. According to the 2018 guidelines of the American Society of Clinical Oncology (ASCO) and the College of American Pathologies (CAP), a HER2-positive IHC score of 3+ is assigned when circumferential membrane staining is complete, intense, and present in >10% of tumor cells. HER2 status may also be determined via in situ hybridization assays (ISH). According to the 2018 guidelines of ASCO/CAP, HER2 is considered amplified if (a) the HER2/chromosome enumeration probe 17 (CEP17) ratio is ≥2.0 and average HER2 copy number is ≥4.0 (group 1); (b) the HER2/CEP17 ratio is ≥2.0 and average HER2 copy number is <4.0 (group 2) with concurrent IHC 3+; (c) the HER2/CEP17 ratio is <2.0 and average HER2 copy number is ≥6.0 (group 3) with concurrent ICH 2+ (equivocal); (d) the HER2/CEP17 ratio is <2.0 and average HER2 copy number is ≥6.0 (group 3) with concurrent IHC 3+; or (e) the HER2/CEP17 ratio is <2.0 with average HER2 copy number ≥4.0 and <6.0 (group 4) with concurrent IHC 3+.

In embodiments, the HER2-positive breast cancer is metastatic breast cancer. As used herein, “metastatic” breast cancer refers to a cancer that has spread beyond the breast and nearby lymph nodes to a different area of the body. Metastasis occurs when cancer cells penetrate the circulatory or lymph systems and travel to distant locations of the body. Metastatic breast cancer may spread to any part of the body. Most often, metastatic breast cancer spreads to the bones, liver, lungs, or brain of the patient. Metastatic breast cancer is also referred to as Stage IV breast cancer.

FIP200, also referred to as RB1CC1, is an autophagy gene that regulates autophagosome formation in cells (Genbank Accession NG_015833.2). In embodiments, human FIP200 comprises a nucleic acid sequence according to SEQ ID NO: 18, or a nucleic acid sequence having at least about 90%, at least about 95%, at least about 98%, at least about 99%, or 100% sequence identity with SEQ ID NO: 18.

In embodiments, human FIP200 protein (Uniprot Ref. Q8TDY2) comprises an amino acid sequence according to SEQ ID NO: 19, or an amino acid sequence having at least about 90%, at least about 95%, at least about 98%, at least about 99%, or 100% sequence identity with SEQ ID NO: 19.

In embodiments, the therapeutic agent that inhibits a nucleic acid that encodes FIP200 is a gene editing agent. Suitable gene editing agents are selected from the group consisting of a Clustered Regularly Interspaced Short Palindromic Repeats (CRISPR)-Cas system, a transcription activator-like effector nuclease (TALEN), and a zing finger nuclease (ZFN).

CRISPR-Cas systems are well known in the art. See, for example, U.S. Pat. No. 10,781,444, issued Sep. 22, 2020 to Zhang, et al.; and U.S. Pat. No. 8,697,359, issued Apr. 15, 2014, to Zhang, et al., each of which is incorporated herein by reference in its entirety. CRISPR-Cas systems include a guide RNA (sgRNA) and a CRISPR-associated endonuclease (Cas protein, such as Cas9, Cas12a, and the like)). The sgRNA is a short synthetic RNA composed of a scaffold sequence necessary for Cas-binding and a user-defined ˜20 nucleotide spacer that defines the genomic target to be modified. CRISPR-Cas systems may be used to knock out a target gene, selectively activate or repress a target gene, purify a region of DNA, image DNA in live cells, and edit DNA and RNA. In embodiments, the sgRNA targets a region of FIP200 in a cancer cell, either in vivo or in vitro. In embodiments, the target region of FIP200 is adjacent to a protospacer adjacent motif (PAM), which serves as a binding signal for the selected Cas protein. In embodiments, CRISPR-Cas systems that target FIP200 may be delivered to a cell via lipid particles, viral vectors, or as messenger RNA (mRNA). See, for example, U.S. Pat. No. 11,352,647, issued Jun. 7, 2022, to Zhang, et al., incorporated herein by reference. In a specific embodiment, the sgRNA comprises SEQ ID NO: 1, or has at least about 90%, at least about 95%, at least about 98%, at least about 99%, or 100% sequence identity with SEQ ID NO: 1.

Transcription activator-like (TAL) effector nucleases (TALENs) are restriction enzymes that can be engineered to cut specific sequences of DNA. Use of TALENs for gene editing is well known in the field. See, for example, U.S. Pat. No. 10,172,880, issued Jan. 8, 2019, to Osborn, et al.; U.S. Pat. No. 8,440,431, issued May 14, 2013, to Voytas, et al.; and U.S. Pat. No. 9,758,775, issued Sep. 12, 2017, to Voytas, et al., each of which is incorporated by reference herein. TALENs have been used to generate non-homologous end joining (NHEJ)-mediated mutations in organisms with high efficiencies, and may also be used to introduce specific insertions in human somatic and pluripotent stem cells using double-stranded donor templates. Various kits and websites are available to assist in identifying TAL effector targets and designing TALENs. See, for example, REAL Assembly TALEN Kit, available from addgene.org. In embodiments, TALENs are designed to target and disrupt a region of FIP200 in a cancer cell, either in vivo or in vitro. In embodiments, TALENs may be delivered to a cell a variety of techniques, including viral vectors, lipid particles, polymers, liposomes, cell-penetrating peptides, and the like.

Zinc finger nucleases (ZFNs) are engineered DNA-binding proteins that facilitate targeted editing of the genome by creating double-strand breaks in DNA at targeted locations. ZFNs leverage endogenous DNA repair machinery to alter genomes. The DNA-binding domains of individual ZFNs typically contain between three and six individual zinc finger repeats and can each recognize between 9 and 18 basepairs. In embodiments, a ZFN targets FIP200 in a cancer cell, either in vivo or in vitro. In embodiments, ZFNs may be delivered to a cell via a variety of techniques, including viral vectors, lipid particles, polymers, liposomes, cell-penetrating peptides, and the like.

In embodiments, the therapeutic agent is a small interfering RNA (siRNA). siRNAs are double-stranded RNA that interfere with the expression of specific genes or complementary nucleotide sequences by degrading mRNA after transcription, thereby preventing translation. siRNAs typically comprise from about 20 to about 24 base pairs of double-stranded RNA, with phosphorylated 5′ ends and hydroxylated 3′ ends and two overhanging nucleotides. In embodiments, an siRNA targets FIP200 in a cancer cell, either in vitro or in vivo.

In embodiments, the therapeutic agent is a short hairpin RNA (shRNA). shRNAs are artificial RNA molecules comprising a hairpin turn that can be used to silence a target gene of interest via RNA interference. In embodiments, an shRNA targets FIP200 in a cancer cell, either in vitro or in vivo. In a specific embodiment, the shRNA comprises SEQ ID NO: 15, SEQ ID NO. 16, or SEQ ID NO: 17, or has at least about 90%, at least about 95%, at least about 98%, at least about 99%, or 100% sequence identity with SEQ ID NO: 15, SEQ ID NO: 16, or SEQ ID NO: 17.

siRNAs and shRNAs may be delivered to a target cell via a variety of techniques, including viral vectors, lipid particles, plasmids, liposomes, nanocarriers, and the like. Such techniques are known in the art and readily available to the skilled person. See, for example Xin, et al., Nano-based delivery of RNAi in cancer therapy, Molecular Cancer 16, 134 (2017).

In embodiments, the methods disclosed herein inhibit FIP200-mediated autophagy in a HER2-positive breast cancer cell of the subject. Specifically, in embodiments, the presently disclosed methods reduce expression of HER2 on the plasma membrane of a HER2-positive breast cancer cell of a subject. In embodiments, the methods decrease HER2 expression on the plasma membrane of the breast cancer cell by diverting HER2 from the plasma membrane to small extracellular vesicles (sEVs), which are then released from the cancer cell. In embodiments, inhibiting FIP200-mediated autophagy shifts the expression of HER2 in the breast cancer cell from the plasma membrane of the cancer cell to sEVs, which are released from the cell. Advantageously reduction of HER2 expression on the surface of the cancer cell via the disclosed methods is effective to mitigate or block metastasis of HER2-positive breast cancer in a subject.

In embodiments, the method further comprises administering to the subject an effective amount of a second therapeutic agent. In embodiments, the second therapeutic agent is a HER2-specific agent. In other embodiments, the second therapeutic agent is an anti-cancer agent. In a specific embodiment, the second therapeutic agent is selected from the group consisting of ado-trastuzumab emtansine (Kadcyla®), fam-trastuzumab deruxtecan (Enhertu®), trastuzumab (Herceptin®), trastuzumab/hyaluronidase (Herceptin Hylecta®), lapatinib (Tykerb®), margetuximab (Margenza®), neratinib (Nerlynx®), pertuzumab, pertuzumab/trastuzumab/hyaluronidase (Phesgo®), tucatinib (Tukysa®), tamoxifen, anastrozole, letrozole, doxorubicin, epirubicin, paclitaxel, radiation therapy, and combinations thereof.

### EXAMPLES

The following examples are given to illustrate various features of the present disclosure and are not intended to be limiting.

### Example 1. Materials and Methods

Mice: fip200f/f, fip200f/KI, MMTV-Cre mice have been described previously. MMTV-Neu mice were obtained from Jackson Lab. The cohorts were maintained on congenic FVB/N genetic background after at least seven generations of backcrossing. For transplantation experiments, 2×10⁵ cells were prepared in PBS and were injected into the inguinal mammary gland fat pad. Tumors were measured by calipers and volume was calculated as (1/2) (length)(width)2. At the endpoint of observation, mice were euthanized and the tumors were excised and weighted.

Cell lines and culture: Primary HER2 tumor cells from FIP200f/f; MMTV-Neu mice (N148 and N418 cells) were isolated and cultured in DMEM/F12 supplemented with 10% FBS, 10 ng/mL EGF, 20 mg/mL insulin, and 50 units/mL penicillin-streptomycin. HER2 tumor cells were then infected with recombinant retroviruses encoding CreERT2 and deletion of Fip200 was induced by culturing with 100 nmol/L 4-hydroxytamoxifen (4-OHT). HeLa and MCF-7 cells were obtained from ATCC and maintained in DMEM supplemented with 10% FBS. FIP200 KO and control WT MEF cells have been described previously.

Antibodies, plasmids and reagents: Antibodies used for immunoblotting were GAPDH (Cell Signaling #2118), FIP200 (Cell Signaling #12436), ATG13 (Cell Signaling #13273), ATGS (Proteintech #10181-2-AP), HER2 (Cell Signaling #2165; Santa Cruz #sc-33684), CD63 (Santa Cruz #sc-5275), ALIX (Cell Signaling #2171), CD81 (Cell Signaling, #10037), p62 (Cell Signaling #5114) and ubiquitin (Santa Cruz #sc-8017). For immunohistochemistry, antibodies used were p62 (Enzo life Sciences #BML-PW9860), cleaved caspase3 (Cell Signaling #9661), Ki67 (Spring Bioscience #m3062). For flow cytometry, antibodies used were HER2-Alexa Fluor-647 (BioLegend #324412), CD24-PerCP (BioLegend #311113), EpCAM-FITC (BioLegend #324203). For gene silencing experiments, siRab27a used were from Santa Cruz (#sc-41834). The plasmids for imaging were purchased from Addgene: DsRed-Rab7 (#12661), mCherry-Rab5 (Q79L) (#35138), mRFP-Rab5 (#14437), mCherry-CD81 (#55012), perbB2-EGFP (#39321), pmTurquoise2-Golgi (#36205) and CD63-pEGFP (#62964). 4-hydroxy-tamoxifen (4-OHT) (#579002), Cycloheximide (CHX) (#C7698), MG132 (#M7449), Bafilomycin A1 (#B1793), 3-Methyladenine (3-MA) (#M9281), chloroquine (CQ) (#C6628) and Dynosore (#D7693) were purchased from Sigma.

CRISPR/Cas9-mediated knockout of FIP200 or ATG13 in HeLa and MCF-7 cells and knockdown of Atg5 in HER2 tumor cells: The pX458 plasmid, pSpCas9(BB)-2A-GFP (PX458) was a gift from Feng Zhang, Addgene plasmid # 48138 was used as the cloning backbone for expressing sgFIP200 and sgATG13. The sgRNA sequences of FIP200 and ATG13 are as follows:

| | | |
|---|---|---|
| SEQ ID NO: 1 | sgFIP200 | CAGGTGCTGGTGGTCAATGG |
| SEQ ID NO: 2 | sgATG13-l | TCACCCTAGTTATAGCAAGA |
| SEQ ID NO: 3 | sgATG13-2 | CAGTCTGTTGTACACCGTGT |
| SEQ ID NO: 4 | sgATG13-3 | GACTGTCCAAGTGATTGTCC |

shRNA mediated knock-down of Atg5 was achieved by lenti-virus transfection into HER2 tumor cells followed by puromycin selection. Lenti-virus were generated by the transfection of psPAX2, pMD2.G and lentiviral plasmids into HEK293T cells. The plasmids of mouse shAtg5 (#1: TRCN0000099432; #2: TRCN0000099430) and scramble control (pLKO.1) were purchased from Sigma.

Western blot: Cells were cultured in 3.5 cm diameter plates (80-90% confluence), washed by PBS buffer, and lysed for 15 min on ice using a RIPA buffer (#C2978, Sigma) containing an anti-protease mix (#PI78415, Thermo Scientific). Protein concentration was measured by BCA assay (#23225, Thermo Scientific). Equal amounts of proteins were subjected to SDS-PAGE and immunoblotting as described previously (Wei et al., 2011).

Immunohistochemistry (IHC) and Immunofluorescence (IF) staining: For histological analysis of tissues, samples were fixed overnight in 4% paraformaldehyde, dehydrated in alcohol gradients, xylene and paraffin before being embedded. Then, they were sectioned (4-μm) and subjected to immuno-histochemistry as previously described (Wei et al., 2011). Inguinal mammary glands were excised at 30 weeks after birth, and whole mounts stained with carmine alum were analyzed, as described previously (Luo et al., 2013). For immuno-cytochemistry, cells cultured on glass coverslips were fixed with methanol at 4° C. for 10 min and blocked with PBS containing 5% goat serum for 1 h. Coverslips were incubated overnight at 4° C. with primary antibody dilutions prepared in PBS containing 5% goat serum. After 3 washes with PBS, coverslips were incubated with Rhodamine-conjugated anti-rabbit and/or FITC-conjugated anti-mouse antibody (Invitrogen) for 2 hrs at room temperature. Cells were washed 3 times with PBS under low light conditions and stained with 1 μg/ml DAPI (Vector Laboratories). All images were generated on Zeiss LSM 710 confocal laser scanning microscope. Quantification of fluorescence intensity and puncta number was performed manually in ImageJ.

Flow cytometry: Unattached dead cells and attached cells were collected after brief trypsinization and stained using antibodies as per manufacturer's protocol. Stained cells were analyzed using FACSAria. Flow cytometry data were analyzed using FlowJo software.

Internalization assay: For live-cell surface HER2 staining, ice-cold culture medium containing Alexa Fluor antihuman Her 2 or mouse IgG1 (control) antibodies were added, and plates incubated in the dark for 1 h on ice and then allowed to internalize at 37 ° C. The level of intracellular HER2 at the end of the assay (3 h) was quantified on individual cells using confocal fluorescence microscopy.

Realtime qPCR: Total RNA was isolated from cells using an RNAeasy kit (Qiagen, #74004) per the manufacturer's instructions. Equal amounts of RNA were then reverse-transcribed using iScript cDNA Synthesis Kit (Bio-rad, #1708891). cDNA samples were then subjected to qRT-PCR analysis with SYBR Green (BioRad, #1725121) in a BioRad CFXConnect (Bio-Rad, Hercules, CA, USA) thermo-cycler. List of primers used are listed as follows:

| | | |
|---|---|---|
| SEQ ID | Atg5 (mouse), | TGTGCTTCGAGATGTGTGGTT |
| NO: 5 | fwd | |
| SEQ ID | Atg5 (mouse), | GTCAAATAGCTGACTCTTGGCAA |
| NO: 6 | rev | |
| SEQ ID | Her2 (rat and | TCCCTGCCAGTCCTGAGACC |
| NO: 7 | mouse), fwd | |
| SEQ ID | Her2 (rat and | GTTGTGAGCGATGAGCATGTA |
| NO: 8 | mouse), rev | |
| SEQ ID | β-Actin, fwd | GGCTGTATTCCCCTCCATCG |
| NO: 9 | | |
| SEQ ID | β-Actin, rev | CCAGTTGGTAACAATGCCATGT |
| NO: 10 | | |
| SEQ ID | HER2 (human), | TGCTGGACATTGACGAGACAGAGT |
| NO: 11 | fwd | |
| SEQ ID | HER2 (human), | AGCTCCCACACAGTCACACCATAA |
| NO: 12 | rev | |
| SEQ ID | GAPDH (human), | CTCCTCCTGTTCGACAGTCAGC |
| NO: 13 | fwd | |
| SEQ ID | GAPDH (human), | CCCAATACGACCAAATCCGTT |
| NO: 14 | rev | |

In vitro cell growth assay: 2,000 tumor cells were plated per well in a 96 well plate. Cell confluency was imaged per hour for 40 hours utilizing the live cell imaging instrument, Incucyte (Essen Biosciences).

sEV isolation and characterization: sEV were isolated from cell culture conditional media followed by serial differential ultracentrifugation as described previously (Wu et al., 2020). sEV size and concentration were determined using Nanoparticle tracking analysis (NTA).

Statistical analysis: Data were plotted as means ±SEM and statistical significance was determined using a two-tailed t-test. For Kaplan—Meier survival plot, a Log-Rank test (Mantel-Cox) was performed. The threshold for significance was p<0.05.

### Example 2. Disruption of FIP200-Mediated Autophagy Abolishes Mammary Tumorigenesis in MTVNeu Mice

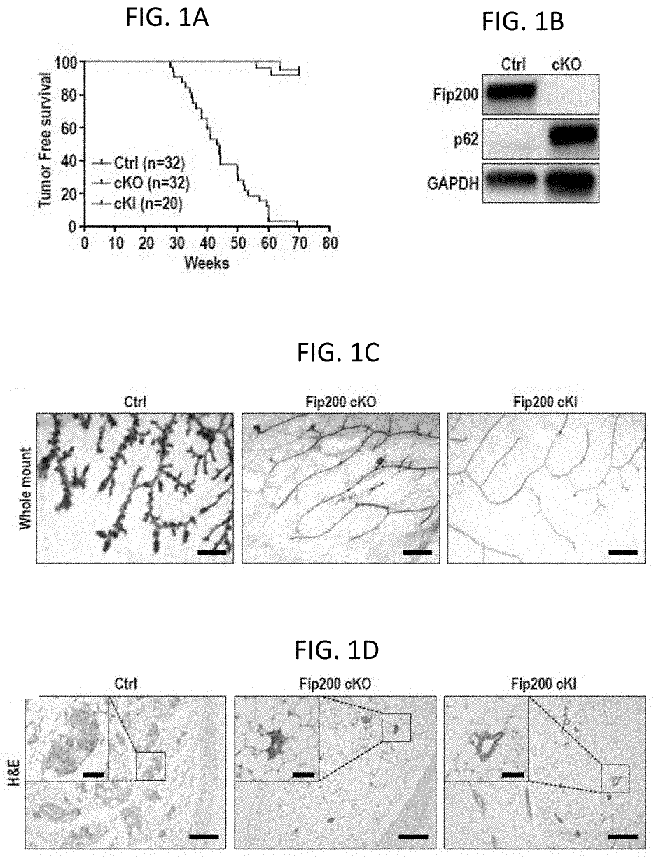

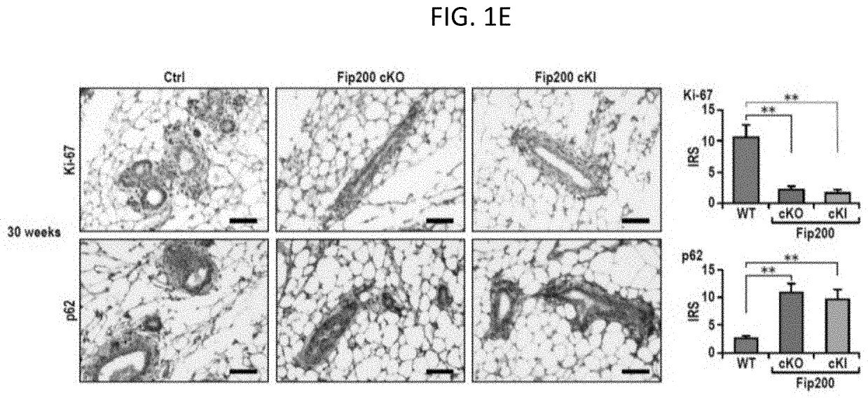

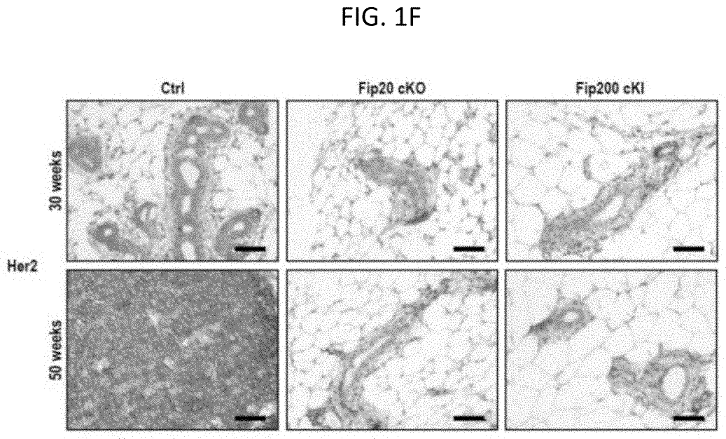

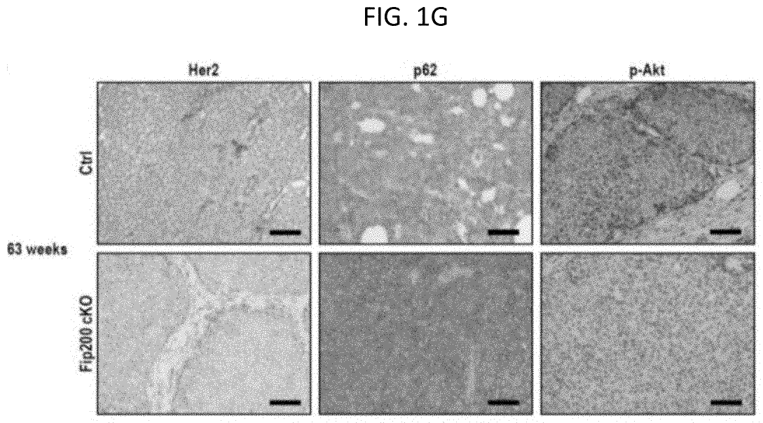

To investigate the role of FIP200 and its canonical autophagy functions in HER2-driven mammary tumors, a mammary epithelial-specific FIP200 knockout was generated in the MMTV-Neu mouse model of breast cancer (fip200f/f; MMTV-Cre; MMTV-Neu, designated as cKO mice) by crossing fip200f/f; MMTV-Cre and MMTV-Neu mice. These mice were then crossed with fip200f/KI mice to prepare mammary epithelialspecific FIP200-4A mutant knock-in mice in the MMTV-Neu model (fip200f/KLMMTVCre; MMTV-Neu, designated as cKI mice), in which mammary tumor cells express only the FIP200-4A mutant allele lacking interaction with Atg13 for autophagy induction. Cohorts of female cKO and cKI mice were compared to litter mate controls (fip200f/+; MMTV-Cre; MMTV-Neu and fip200+/+; MMTV-Cre; MMTV-Neu, designated as Ctrl mice) for mammary formation monitored by physical palpation. FIG. 1A shows that about half of Ctrl mice developed palpable tumors by the age of 43 weeks (T1/2=43 weeks), with tumors developing as early as 29 weeks of age, and all 32 out of 32 (100%) mice having tumors by 70 weeks. In contrast, only 2 out of 32 (6.25%) cKO mice and 1 out of 20 (5%) cKI mice developed mammary tumors by 70 weeks of age. Thus, unlike previous reports that FIP200 ablation moderately delayed tumor onset for MMTV-PyMT (T1/2=9 weeks vs 8 weeks) (Wei et al., 2011) and BRCA1-deficient model (T1/2=27 weeks vs 22 weeks) (Yeo et al., 2018), FIP200 ablation or blocking FIP200-mediated autophagy substantially abolished mammary tumorigenesis in MMTV-Neu model. Tumor lysates were prepared from cKO mice and Ctrl mice at 63 weeks of age for Western blotting to verify deletion of FIP200. As expected, FIP200 expression was diminished in tumors from cKO mice compared to Ctrl mice (FIG. 1B).

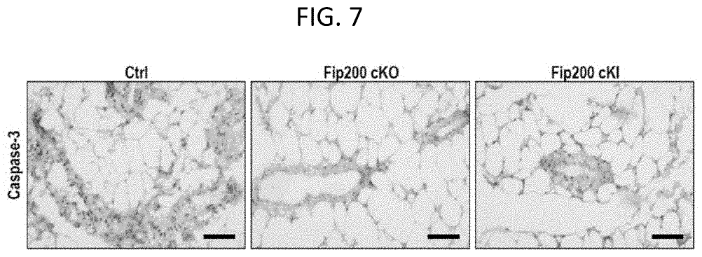

Further, increased accumulation of p62 in cKO tumors was observed relative to Ctrl tumors, consistent with reduced autophagy activity. Collectively, these results suggest that FIP200 and specifically its canonical autophagy function is required for HER2-driven mammary tumorigenesis. Whole mount staining of mammary glands from mice at an earlier age (i.e. 30 weeks) was carried out to evaluate the effect of blocking FIP200-mediated autophagy on hyperplasia of MMTV-Neu mice. The epithelial surfaces displayed extensive lobulo-alveolar formation throughout the mammary ductal trees (FIG. 1C). In contrast, mammary glands from cKO and cKI mice at the same age showed apparently normal ductal structures with minimal alveolar budding. Moreover, H&E staining of mammary gland sections showed expanded lobulo-alveolar structures in Ctrl mice, whereas ductal structures were prevalent in cKO and cKI mice (FIG. 1D). Tumor cell proliferation was examined by Ki-67 staining of the sections. A high fraction of mammary epithelial cells showed positive Ki-67 staining in Ctrl mice, which was significantly decreased in both cKO and cKI mice, suggesting that their proliferation was suppressed upon blockade of autophagy in these cells (FIG. 1E, top panels). However, no appreciable level of apoptosis was detected in any of the samples by IHC using cleaved caspase 3 (FIG. 7). As expected, higher p62 staining was observed in the mammary epithelial cells of cKO and cKI mice relative to those in Ctrl mice, consistent with the decreased autophagy in these cells (FIG. 1E, bottom panels). Together, these results suggest that disruption of FIP200-mediated autophagy reduced mammary tumor cell proliferation to inhibit mammary tumorigenesis in MMTV-Neu mice.

### Example 3. Autophagy Blockade Decreases HER2 Levels in Mammary Tumor Cells

To explore potential mechanisms by which blockade of FIP200-mediated autophagy abolished HER2-driven mammary tumorigenesis in vivo, the effect of FIP200 deletion on HER2 expression by IHC was explored (FIG. 1F). As expected, HER2 was detected in the mammary epithelia of Ctrl mice at 30 weeks of age, and throughout mammary tumors in 50-week-old Ctrl mice. In contrast, lower levels of HER2 were found in mammary epithelial cells of cKO or cKI mice at 30 and 50 weeks of age, suggesting that blocking FIP200-mediated autophagy decreased HER2 levels, which may compromise mammary tumorigenesis in MMTV-Neu mice. Comparison of mammary tumors from Ctrl and cKO mice at 63 weeks of age verified the significantly decreased HER2 levels upon FIP200 deletion even for the tumors developed in the small fraction of cKO mice (i.e., 2 out of 32 mice) (FIG. 1G). Consistent with Western blotting results (see FIG. 1B), p62 levels are increased in cKO tumors compared to Ctrl tumors. Moreover, analysis of phosphorylated Akt showed that the activation of a major HER2 downstream pathway Akt signaling was significantly decreased in cKO tumors relative to Ctrl tumors, consistent with reduced HER2 levels after FIP200 deletion.

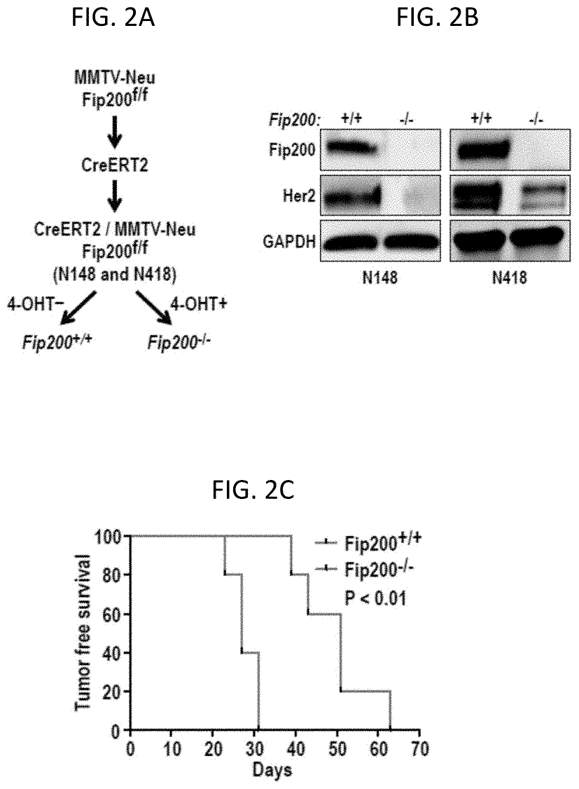

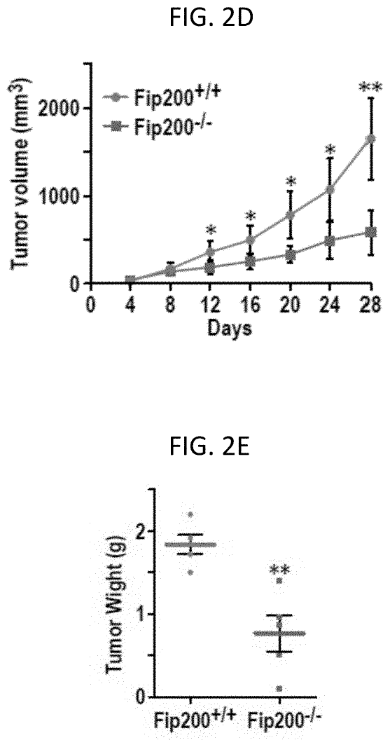

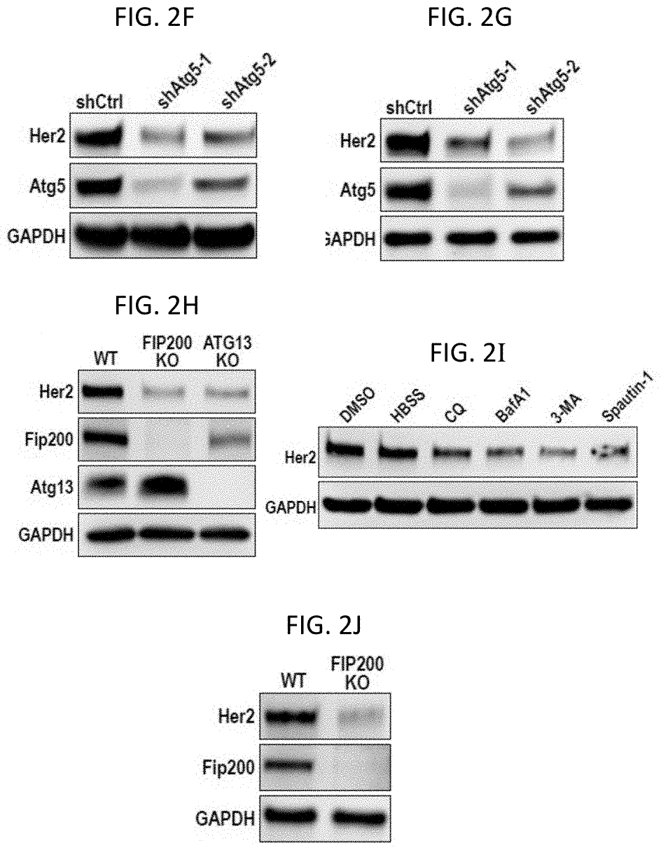

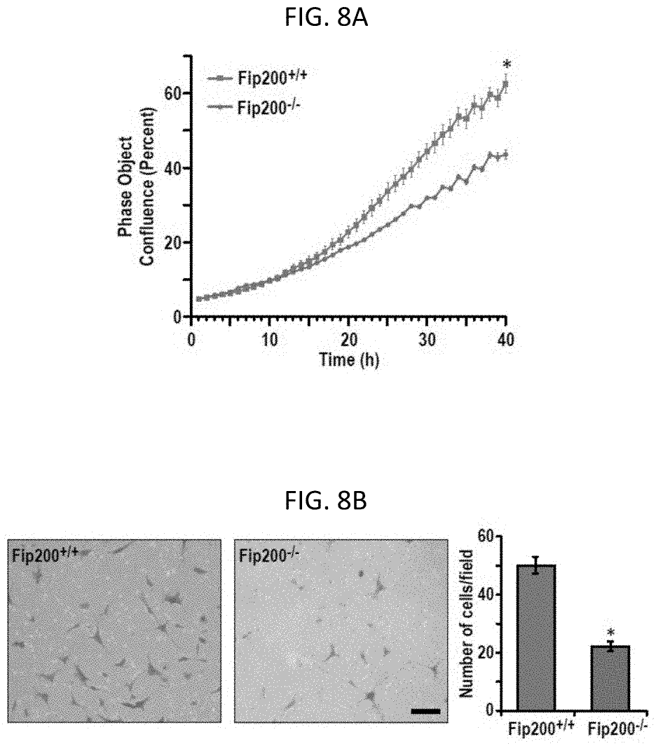

The extremely small fraction of cKO mice developing tumors in the time frame (i.e. >70 weeks) that the majority of Ctrl mice were still viable limit further mechanistic studies requiring sufficient numbers in cohorts for statistical significance. To overcome this limitation, two independent spontaneously immortalized mammary tumor cells were developed from Fip200f/f; MMTV-Neu mice, and then infected with recombinant retroviruses encoding CreERT2 and a luciferase marker (N148 and N418 cells, collectively designated as HER2 tumor cells) (FIG. 2A). Upon treatment with 4-hydroxytamoxifen (4-OHT) to induce Cre recombinase expression and consequent deletion of the floxed Fip200 allele, FIP200 expression was substantially reduced in both lines of HER2 tumor cells (FIG. 2B). These cells are designated as Fip200−/− and Fip200+/+ tumor cells (with and without 4-OHT treatment), respectively, and used for further studies. Consistent with results in tumor cells from cKO mice (see FIG. 1G), FIP200 deletion also led to the reduced HER2 levels in both Fip200−/− tumor cells. Moreover, in vitro assays showed that Fip200−/− tumor cells exhibited decreased cell proliferation and migration compared to Fip200+/+ tumor cells (FIGS. 8A, 8B). To further validate Fip200−/− tumor cells as a good model mimicking the interesting in vivo phenotypes observed for cKO mice in vivo, equal number of Fip200+/+ and Fip200−/− tumor cells were orthotopically transplanted into syngeneic FVB mice and observed for tumor development and progression. Similar to our observations in cKO mice, tumor formation in recipient FVB mice transplanted with Fip200−/− tumor cells was significantly compromised compared with those transplanted with Fip200+/+ tumor cells (FIG. 2C). Furthermore, Fip200−/− tumor cells also showed significantly decreased growth relative to Fip200+/+ tumor cells in the recipient mice (FIGS. 2D and 2E). Together, these results suggest that FIP200 ablation reduced HER2-driven mammary tumor development and progression by decreasing HER2 levels in tumor cells.

To further evaluate the role of autophagy in the regulation of HER2 levels, shRNA was used to knockdown expression of another essential autophagy gene, Atg5, in the two lines of HER2 tumor cells (FIGS. 2F and 2G). Similar to FIP200 deletion, autophagy inhibition by Atg5 knockdown also reduced HER2 levels in both of these mammary tumor cells. Disruption of autophagy by CRISPR-Cas9 mediated knockout of FIP200 or another essential autophagy gene ATG13 also reduced HER2 levels in HeLa cells (FIG. 2H). Consistent with these genetic manipulations, blocking autophagy with several different inhibitors also reduced levels of HER2 in HeLa cells (FIG. 2I). FIP200 KO MEFs showed reduced HER2 levels compared to control MEFs (FIG. 2J). These results suggest that autophagy inhibition reduced HER2 levels in a variety of cells. Further, decreased HER2 levels upon FIP200 deletion was due to autophagy blockade, rather than the loss of other activities of FIP200, in HER2-driven mammary tumor cells, which was consistent with the similar phenotypes of cKO and cKI mice.

### Example 4. Intracellular Accumulation of HER2 and its Reduced Levels on the Plasma Membrane in Autophagy-Deficient Tumor Cells

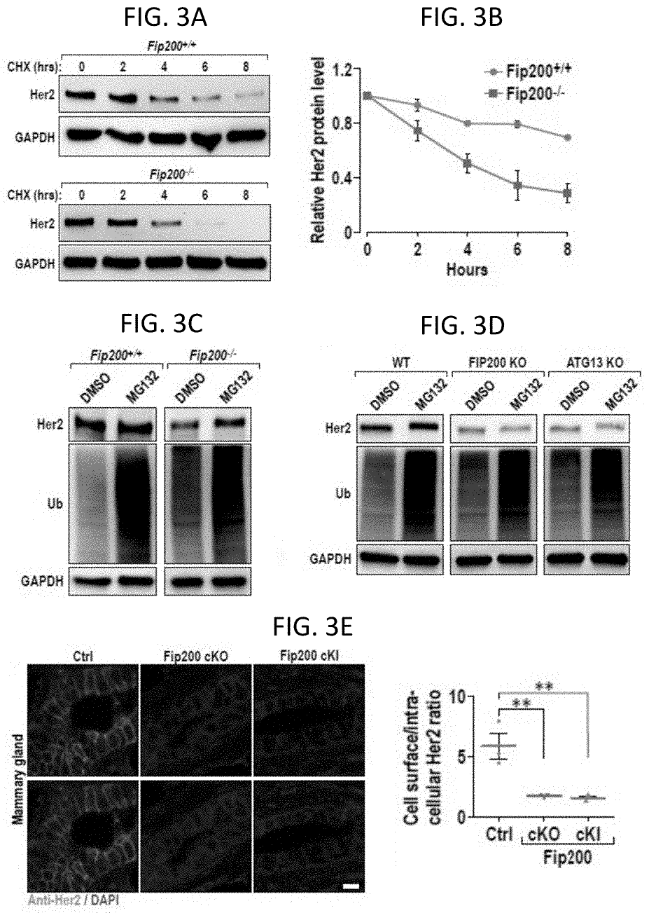

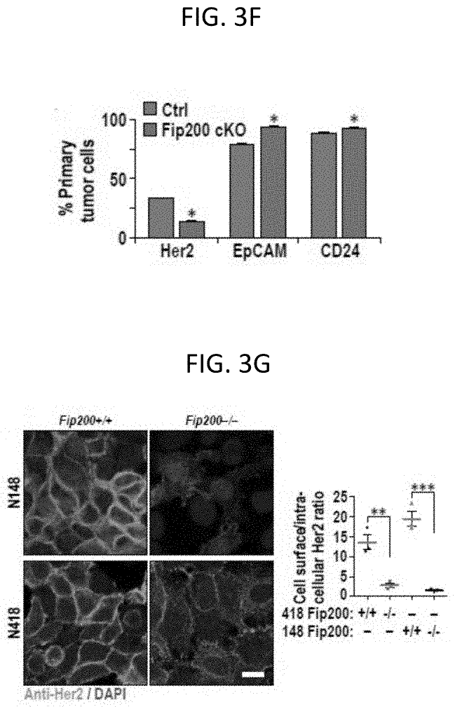

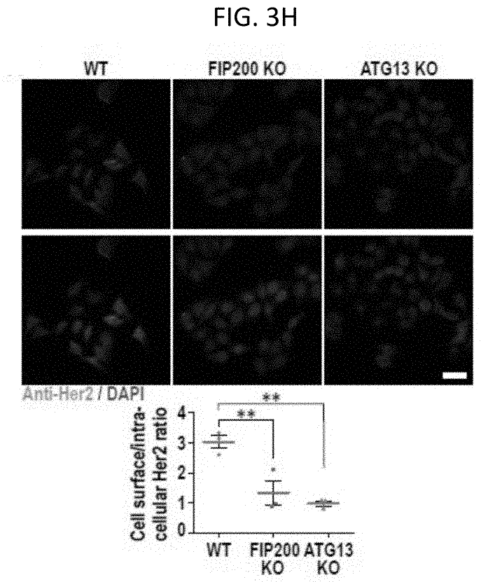

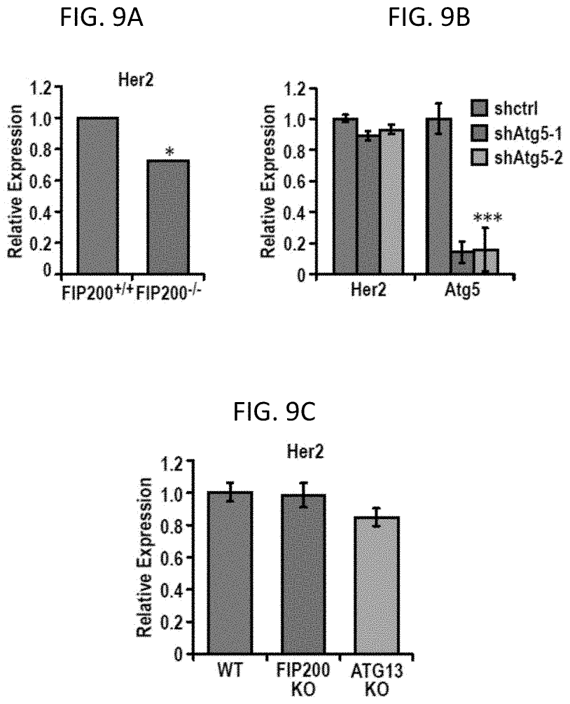

To further investigate regulation of HER2 by autophagy, mRNA levels of HER2 in mammary tumor and HeLa cells with or without autophagy inhibition were examined. A slight reduction of HER2 mRNA level in Fip200−/− tumor cells was observed compared to Fip200+/+ tumor cells (FIG. 9A). However, knockdown of Atg5 in HER2 tumor cells did not affect HER2 mRNA level in these cells (FIG. 9B). Moreover, deletion of FIP200 or ATG13 did not reduce mRNA levels of HER2 in HeLa cells (FIG. 9C). Thus, autophagy inhibition unlikely reduced HER2 expression at transcriptional or mRNA stability levels. Next, HER2 protein stability was analyzed and a decreased half-life of HER2 protein in Fip200−/− tumor cells (˜3 hrs) relative to Fip200+/+tumor cells (˜5 hrs) (FIGS. 3A and 3B) was observed. MG132 was used to inhibit proteasome-mediated protein degradation and its effect on HER2 protein levels in autophagy deficient HER2 tumor and HeLa cells was determined. Surprisingly, however, MG132 did not significantly reverse the reduced levels of HER2 protein in Fip200−/− tumor cells (FIG. 3C) or FIP200 and ATG13 KO HeLa cells (FIG. 3D). These results suggest that autophagy inhibition reduced tumor cell-associated HER2 by mechanisms other than inducing its increased degradation through proteasomes.

To explore such potential alternative mechanisms for the reduced HER2 in autophagy deficient cells, cell surface expression of HER2 was examined, given that its oncogenic activity relies on its complex formation with other HER family members to trigger downstream signaling pathways such as Akt activation (which was compromised in cKO tumors, see FIG. 1G). Immunofluorescent staining of mammary gland sections from 30-week-old mice showed that HER2 was predominantly localized on the cell surface in Ctrl mice, but much reduced in the plasma membrane of cKO and cKI mice (FIG. 3E), which were consistent with their significantly decreased tumorigenesis (see FIG. 1A). Interestingly, despite the decreased amount of HER2 protein (see FIG. 1F) as well as the corresponding reduction on the cell surface, we found some diffuse presence of HER2 in the cytoplasm of mammary gland cells from cKO and cKI mice, suggesting potentially altered intracellular trafficking of HER2 upon autophagy inhibition. Flow cytometry analysis of mammary tumor cells isolated from 63-week-old cKO and Ctrl mice verified the reduced HER2 expression on the surface of tumor cells after Fip200 deletion, while the levels of epithelial markers Cd24 and EpCAM were slightly increased in cKO tumor cells (FIG. 3F).

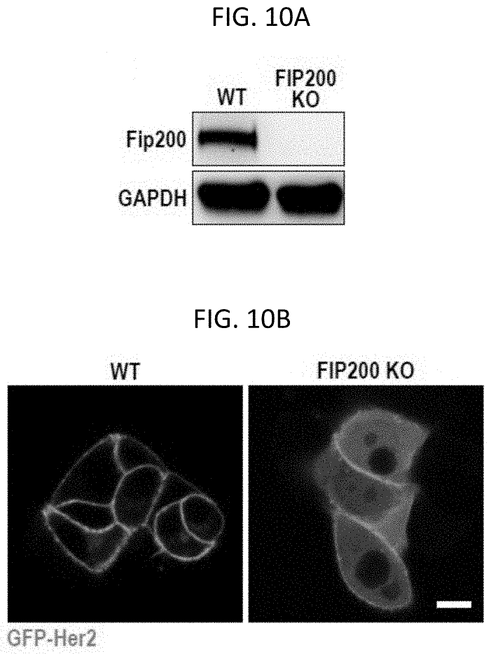

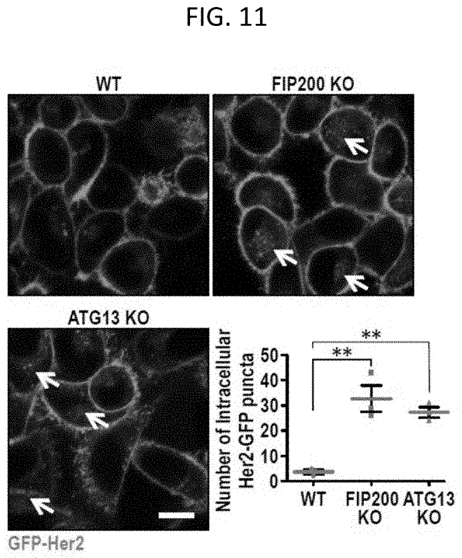

To further examine possible changes in intracellular trafficking of HER2 upon autophagy inhibition, HER2 distribution was examined in established mammary tumor cells and HeLa cells by both immunofluorescent staining and ectopic expression of GFP-HER2 fusion protein. Similar to results from cKO mice, both Fip200−/− tumor cells showed reduced HER2 on the cell surface, but some level of intracellular accumulation, relative to Fip200+/+ tumor cells (FIG. 3G). Similarly, HeLa cells with FIP200 or ATG13 KO showed reduced HER2 on the plasma membrane (FIG. 3H). Moreover, ectopic expression of GFP-HER2 also showed intracellular accumulation in MCF-7 mammary tumor cells with FIP200 KO, as well as HeLa cells with FIP200 or ATG13 KO, but not respective control MCF-7 and HeLa cells (FIGS. 10 and 11). Together, these results indicate that autophagy inhibition may change HER2 intracellular trafficking to decrease its levels on the plasma membrane and signaling for tumorigenesis.

### Example 5. Autophagy Inhibition Alters HER2 Intracellular Trafficking to Divert its Transport from the Golgi to Endosomes Instead of the Plasma Membrane

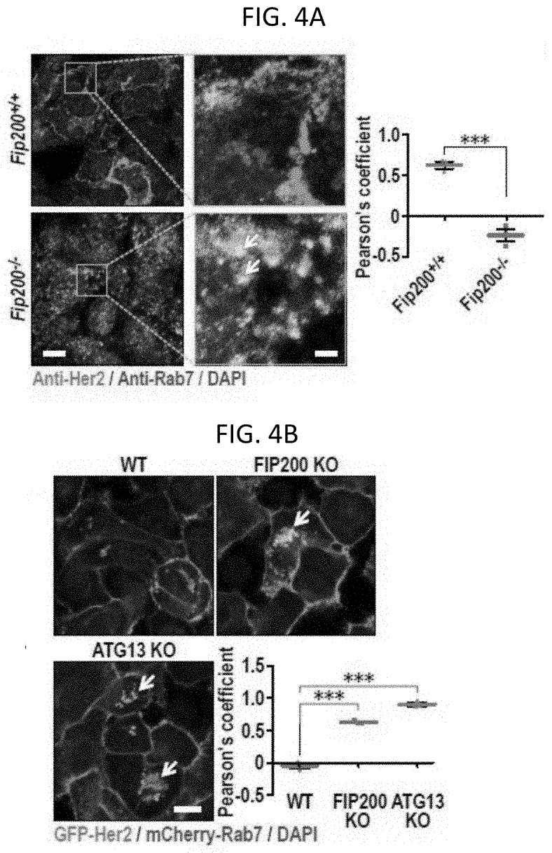

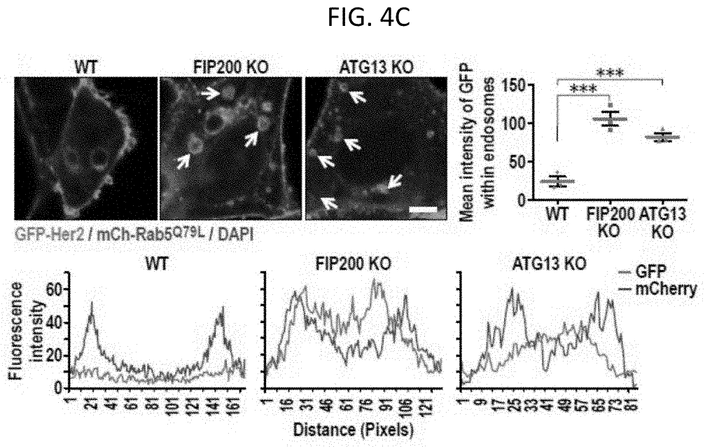

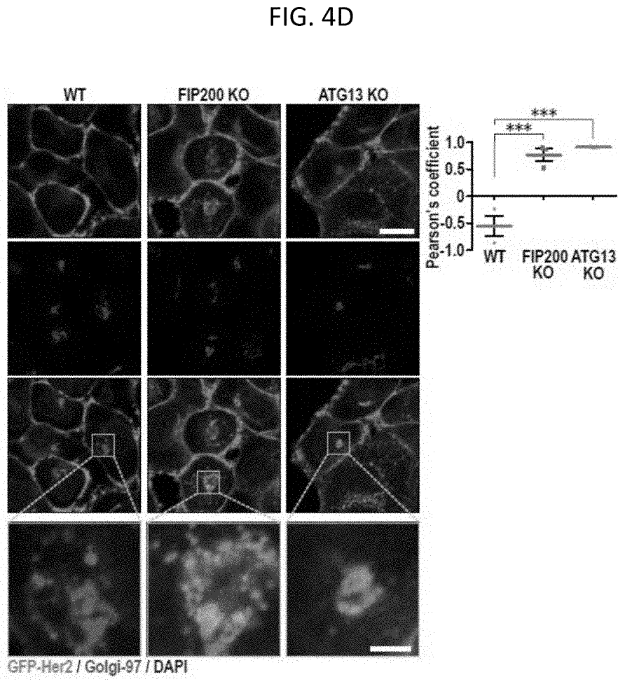

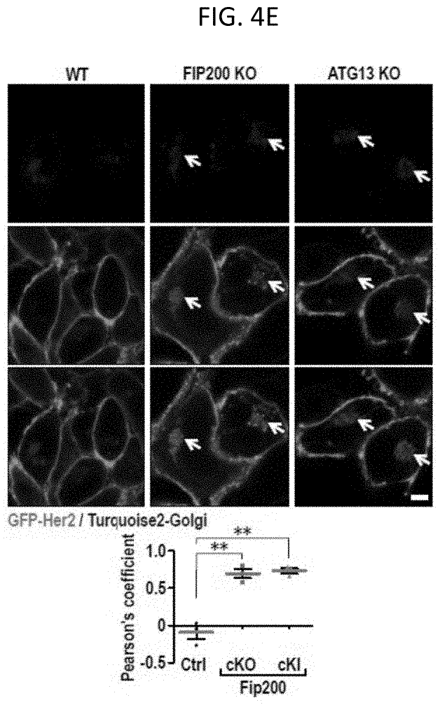

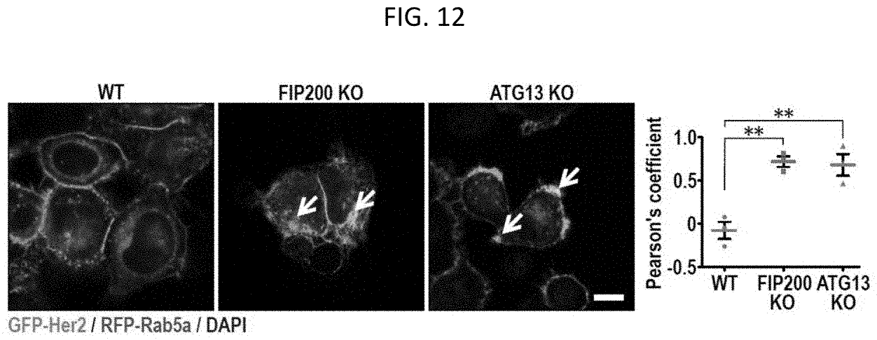

Endocytosis is a major mechanism for the regulation of cell surface levels of many receptor tyrosine kinases including HER2. Experiments were carried out to determine whether the increased intracellular accumulation of HER2 in autophagy deficient tumor cells was localized in endosomes using markers for early and late endosomes. Double label immunofluorescent staining showed co-localization of intracellularly accumulated HER2 with a late endosome marker Rab? in Fip200−/− tumor cells but not Fip200+/+ tumor cells (FIG. 4A). Similarly, co-expression of GFP-HER2 and mCherry-Rab7 showed co-localization of HER2 in late endosomes of HeLa cells with FIP200 or ATG13 KO, but not control HeLa cells (FIG. 4B). Co-expression of mCherry-Rab5, an early endosome marker, with GFP-HER2 showed some co-localization of HER2 in this compartment in HeLa cells with FIP200 or ATG13 KO (FIG. 12). To further evaluate increased HER2 in the endocytic pathway upon autophagy block, GFP-HER2 was co-expressed with mCherry-Rab5Q79L encoding a mutant constitutively active Rab5, which impairs early to late endosome transition and causes enlarged early endosomes in these cells. Interestingly, more extensive trapped GFP-HER2 was observed in the enlarged early endosomes marked by mCherry-Rab5Q79L in autophagy-deficient HeLa cells with FIP200 or ATG13 KO, but not control HeLa cells (FIG. 4C). Together, these results demonstrate accumulation of HER2 in endocytic vesicles after autophagy blockade, suggesting increased endocytosis of HER2, which may contribute to reduced HER2 levels on the cell surface.

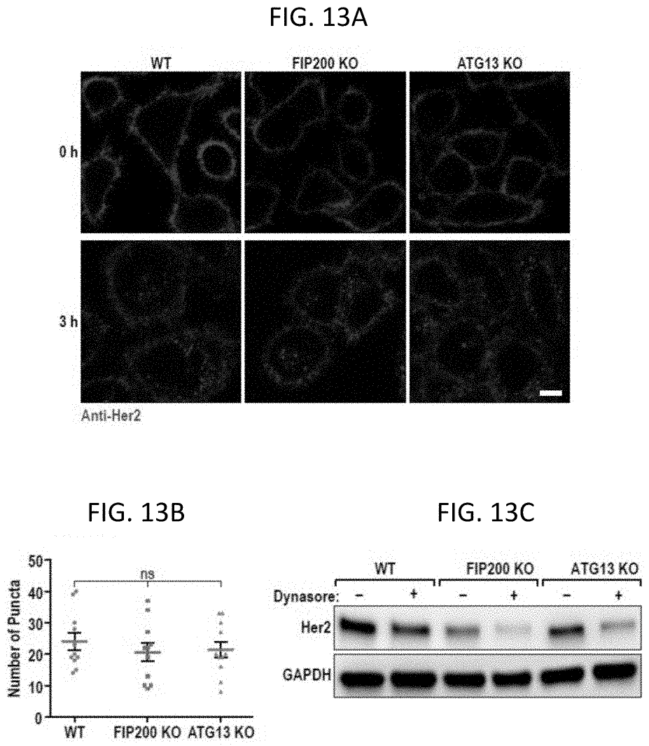

To test the above possibility directly, endocytosis of HER2 in HeLa cells with or without autophagy blockade was examined. Labeling of un-permeabilized live cells showed reduced HER2 on the surface of FIP200 and ATG13 KO HeLa cells relative to control HeLa cells (FIG. 13A, top panels). Interestingly, however, similar amount of cell surface HER2 were endocytosed after 3 hrs, as measured by the number of internalized HER2 puncta (i.e., endosomes) between autophagy deficient FIP200 or ATG13 KO HeLa cells and control HeLa cells (FIG. 13A, bottom panels, FIG. 13B). Moreover, inhibition of endocytosis by Dynasore did not rescue the decreased level of HER2 in FIP200 and ATG13 KO HeLa cells (FIG. 13C). In addition to endocytosis from the plasma membrane, proteins from the Golgi could be transported through endosomes to the lysosome or other compartments. Interestingly, intracellular accumulation of GFP-HER2 was detected in the Golgi of FIP200 and ATG13 KO HeLa cells, but not control HeLa cells, as measured by co-staining with Golgi-97 as a marker (FIG. 4D). Co-expression of mTurquoise2-beta-1,4-galactosyltransferase 1 (which constitutively expresses in the Golgi) in these cells further supported accumulation of HER2 in the Golgi of FIP200 and ATG13 KO HeLa cells (FIG. 4E). Together, these results indicate that autophagy deficiency alters HER2 trafficking for accumulation in early and late endosomes caused by diverting its transport from the Golgi to endosomes rather than to the plasma membrane as in autophagy-competent cells.

### Example 6. Autophagy Inhibition Increases HER2 Release Through Small Extracellular Vesicles from Tumor Cells Leading to Reduced Cell Surface Levels and Tumorigenesis

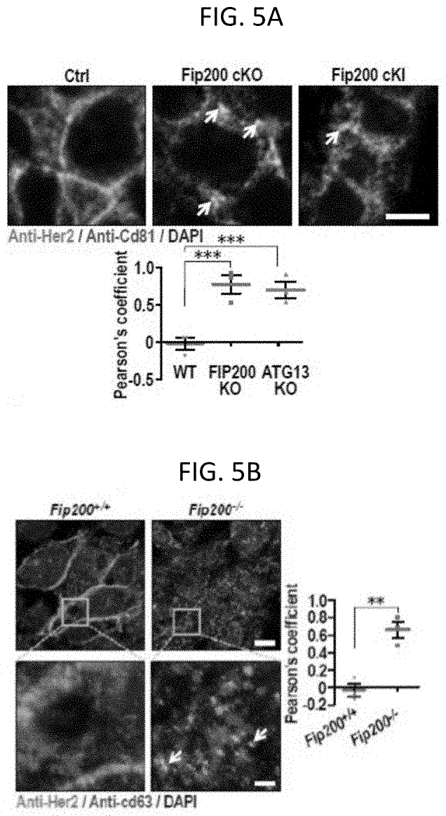

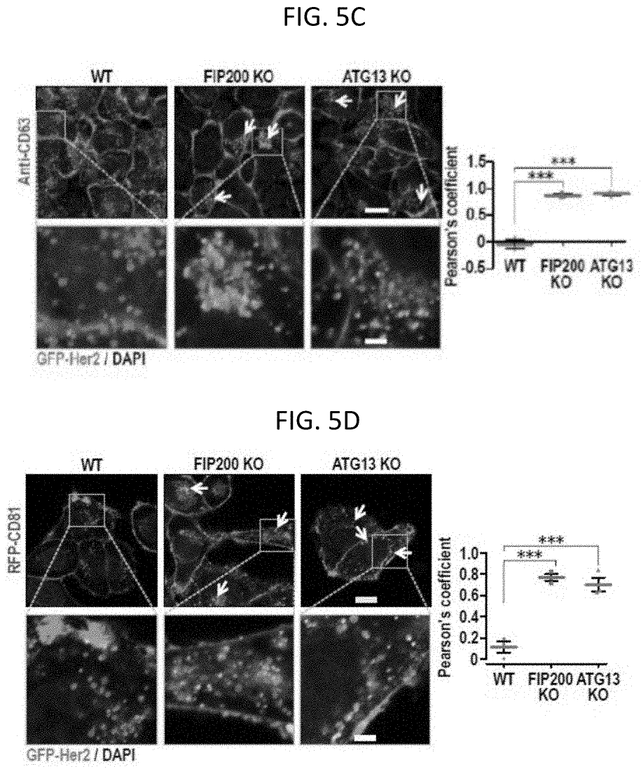

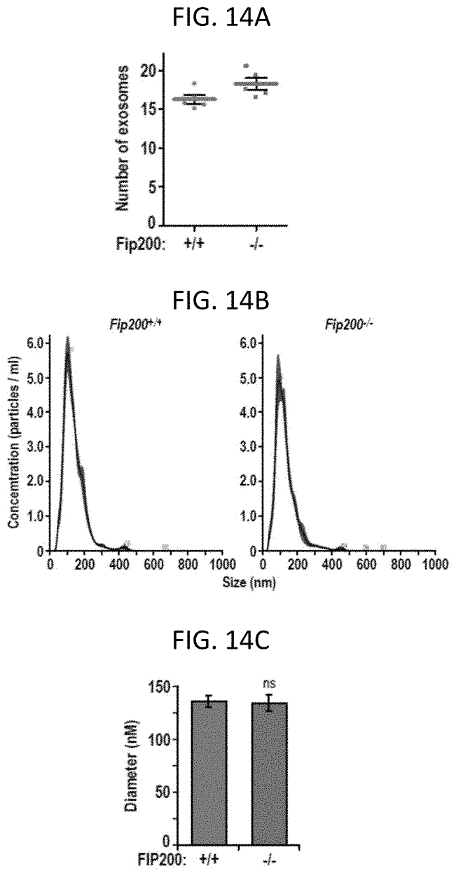

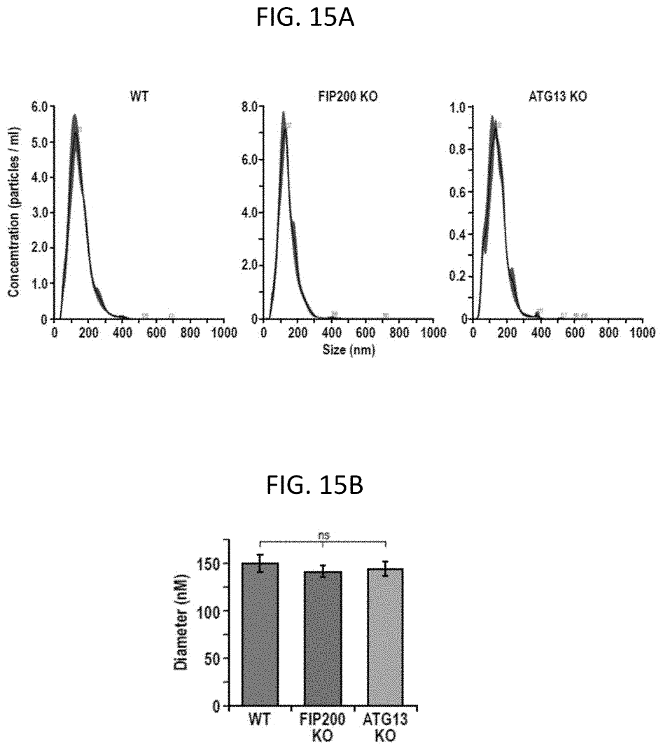

In addition to fusion with autophagosomes/lysosomes for cargo degradation, some late endosomes are also called multivesicular bodies (MVBs) characterized by the presence of intraluminal vesicles (ILVs) that can be released from the cells as small extracellular vesicles (sEVs) by exocytosis. Because the data suggests that inhibition of lysosomal degradation by chloroquine or bafilomycin Al also reduced HER2 levels rather than increased or maintained its level (see FIG. 2I), experiments were carried out to determine whether blocking autophagy leads to HER2 loss through exocytosis after FIP200 deletion, given the increased intracellular accumulation in early and late endosomes (see FIGS. 4B and 4C). Indeed, double label immunofluorescent staining showed co-localization of accumulated HER2 with CD81, a marker for ILV, in mammary epithelial cells of cKO and cKI mice (FIG. 5A). Similarly, co-localization of intracellularly accumulated HER2 was detected with another ILV marker CD63 in Fip200−/− tumor cells (FIG. 5B). Moreover, ectopically expressed GFP-HER2 showed extensive co-localization with both CD63 and co-expressed RFP-CD81 in HeLa cells with FIP200 or ATG13 KO, but not control HeLa cells (FIGS. 5C and 5D). Collectively, these data indicate that intracellular HER2 in autophagy deficient cells was transported through endosomal compartments and accumulated within ILVs. While not desiring to be bound by theory, from ILVs, it is possible that HER2 was subsequently released from these cells in sEVs. To test directly the hypothesis that deceased HER2 in autophagy impaired tumor cells was due to its diversion to MVBs and released in sEVs, culture supernatants from Fip200+/+ and Fip200−/− tumor cells were subjected to serial differential ultra-centrifugation to collect sEVs (at 100,000g). Western blot analysis of lysates from sEVs showed a significant increase of HER2 in sEVs from Fip200−/− tumor cells relative to that from Fip200+/+ tumor cells (FIG. 6A). Some increase of Alix (FIG. 6A), a marker for sEV, as well as total amount of proteins (FIG. 6B) in sEV lysates from Fip200−/− tumor cells were noted, suggesting a possible increased amount of sEV from the same number of cells after autophagy inhibition. However, direct measurement of sEV showed only a slight increase in the number of sEV from Fip200−/− tumor cells and no difference in their size between Fip200+/+ and Fip200−/− tumor cells (FIG. 14), suggesting a preferential enrichment of HER2 in sEV for release from tumor cells after autophagy blockade. Indeed, an increased amount of HER2 in sEV lysates from Fip200−/− tumor cells was observed compared to that from Fip200+/+ tumor cells, when equal amount of proteins was examined (FIG. 6C). Similar analysis of HeLa cells showed that FIP200 and ATG13 KO also increased HER2 release from cells through sEVs in comparison to control HeLa cells, as measured by both equal amount of protein lysates (FIG. 6D) or equal number of sEVs (FIG. 6E). Moreover, increases of the number of sEV in HeLa cells with FIP200 and ATG13 KO were observed relative to control HeLa cells (FIG. 6F), although no difference in sEV size was detected for these cells after autophagy blockade (FIG. 15). Consistent with this, a significantly increased number of ILVs were found within MVBs marked by CD63 in FIP200 and ATG13 KO HeLa cells compared to control HeLa cells (FIG. 6G), further supporting that both increased sEV biogenesis and preferential enrichments of HER2 into sEV may contribute to the increased release of HER2 and their decrease in tumor cells after autophagy blockade. Lastly, siRNA was used to knockdown Rab27a, which is required for fusion of MVBs with the plasma membrane to release ILVs from cells as sEVs. Blocking sEV release by silencing Rab27a restored HER2 levels in both FIP200 and ATG13 KO HeLa cells (FIG. 6H). Taken together, these results demonstrate that autophagy inhibition increased HER2 release through sEVs by altering its intracellular trafficking, leading to reduced HER2 expression on the surface of tumor cells and tumorigenesis in cKO mice.

### Example 7. Effects of Autophagy Inhibition on Neu Levels from Serum sEVs and Metastatic Colonization

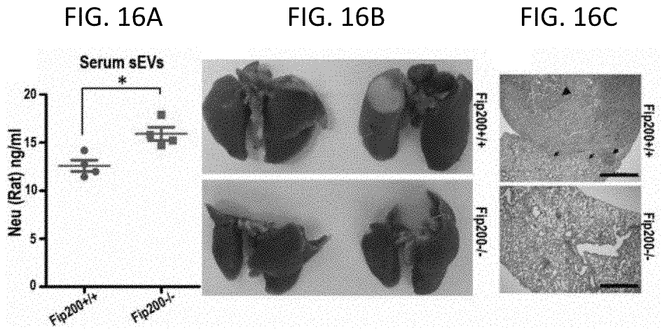

Blood samples from recipient FVB mice were collected at 28 days after transplantation with Fip200+/+ or Fip200−/− tumor cells. sEVs prepared from the blood samples were examined for Neu protein (i.e., HER2 oncogenic driver in MMTV-Neu mice model from which these tumor cells were derived). Results showed increased Neu released to the blood via sEVs from tumors induced by Fip200−/− tumor cells (FIG. 16A), consistent with results from analysis of these tumor cells in vitro. Experiments were carried out to determine the effect on experimental metastatic activity of mammary tumor cells using tail vein injection by FIP200 deletion and altered HER2 trafficking. Results indicated that Fip200−/− tumor cells exhibited significantly reduced metastatic activity compared to Fip200+/+ tumor cells (FIGS. 16B, 16C). Together, these results demonstrate a new function of autophagy in the regulation of HER2 trafficking for mammary tumorigenesis and metastasis, supporting autophagy inhibition as a distinct treatment strategy that may synergize with current anti-HER2 agents.

### Example 8. Targeted FIP200 Silencing Using shRNAs

A lipid nanoparticle carrier, a plasmid, or a viral vector is used to systemically administer a therapeutic shRNA that silences FIP200 to a human subject having HER2-positive breast cancer or to a mouse model of HER2-positive breast cancer, such as an MMTV-Neu mouse model. Exemplary shRNAs are set forth as follows:

| | | |
|---|---|---|
| SEQ ID | FIP200 | CCGGCCAAGGATTATTCGACCATTTCTCG |
| NO: 15 | shRNA | AGAAATGGTCGAATAATCCTTGGTTTTT |
| | (human) | |
| SEQ ID | FIP200 | GCTGAATTTCAGTGCTTAGAA |
| NO: 16 | shRNA | |
| | (mouse) | |
| SEQ ID | FIP200 | CCAACTTTAACACAGTCTTAA |
| NO: 17 | shRNA | |
| | (mouse) | |

Treatment with the therapeutic shRNA inhibits FIP200-mediated autophagy, reduces the amount of HER2 expressed on the plasma membrane of cancer cells, increases the release of sEVs carrying HER2 on their vesicular membranes, and inhibits breast cancer metastasis and tumorigenesis in the treated subject.

Patents, applications, and publications mentioned in the specification are indicative of the levels of those skilled in the art to which the invention pertains. These patents and publications are incorporated herein by reference to the same extent as if each individual application or publication was specifically and individually incorporated herein by reference.

The foregoing description is illustrative of particular embodiments of the invention, but is not meant to be a limitation upon the practice thereof. The following claims, including all equivalents thereof, are intended to define the scope of the invention.

1. A method of treating human epidermal growth factor receptor 2 (HER2)-positive breast cancer in a subject in need thereof, the method comprising administering to the subject an effective amount of a therapeutic agent that inhibits a nucleic acid that encodes FAK family-interacting protein of 200 kDa (FIP200).

2. The method according to claim 1, wherein the HER2-positive breast cancer is metastatic.

3. The method according to claim 1, wherein the therapeutic agent is a gene editing agent.

4. The method according to claim 3, wherein the gene editing agent is selected from the group consisting of a CRISPR-Cas system, a transcription activator-like effector nuclease (TALEN), and a zing finger nuclease (ZFN).

5. The method according to claim 1, wherein the therapeutic agent is an siRNA or an shRNA,

6. The method according to claim 5, wherein the shRNA is selected from the group consisting of SEQ ID NO: 15, SEQ ID NO: 16, and SEQ ID NO: 17.

7. The method according to claim 1, wherein the method inhibits FIP200-mediated autophagy in a HER2-positive breast cancer cell of the subject.

8. The method according to claim 1, wherein the method reduces expression of HER2 on a plasma membrane of a HER2-positive breast cancer cell of the subject.

9. The method according to claim 8, wherein the method stimulates release of small extracellular vesicles (sEVs) from a HER2-positive breast cancer cell of the subject.

10. The method according to claim 9, wherein a vesicular membrane of the sEV comprises HER2.

11. The method according to claim 1, further comprising administering to the subject an effective amount of a second therapeutic agent.

12. The method according to claim 12, wherein the second therapeutic agent is selected from the group consisting of ado-trastuzumab emtansine, fam-trastuzumab deruxtecan, trastuzumab, trastuzumab/hyaluronidase, lapatinib, margetuximab, neratinib, pertuzumab, pertuzumab/trastuzumab/hyaluronidase, tucatinib, tamoxifen, anastrozole, letrozole, doxorubicin, epirubicin, paclitaxel, radiation therapy, and combinations thereof.

13. A method of inhibiting metastasis of human epidermal growth factor receptor 2 (HER2)-positive breast cancer in a subject in need thereof, the method comprising administering to the subject an effective amount of a therapeutic agent that inhibits FAK family-interacting protein of 200 kDa (FIP200)-mediated autophagy.

14. The method according to claim 13, wherein the therapeutic agent inhibits a nucleic acid that encodes FIP200.

15. The method according to claim 14, wherein the therapeutic agent is a gene editing agent.

16. The method according to claim 15, wherein the gene editing agent is selected from the group consisting of a CRISPR-Cas system, a transcription activator-like effector nuclease (TALEN), and a zing finger nuclease (ZFN).

17. The method according to claim 16, wherein the CRISPR-Cas system comprises a CRISPR-associated endonuclease and a guide RNA (sgRNA), wherein the sgRNA targets FIP200.

18. The method according to claim 14, wherein the therapeutic agent is an siRNA or an shRNA,

19. The method according to claim 17, wherein the shRNA is selected from the group consisting of SEQ ID NO: 15, SEQ ID NO: 16, and SEQ ID NO: 17.

20. The method according to claim 13, wherein the method reduces expression of HER2 on a plasma membrane of a HER2-positive breast cancer cell of the subject.

21. The method according to claim 20, wherein the method stimulates release of small extracellular vesicles (sEVs) from a HER2-positive breast cancer cell of the subject.

22. The method according to claim 21 wherein a vesicular membrane of the sEV comprises HER2.

23. The method according to claim 13, further comprising administering to the subject an effective amount of a second therapeutic agent.

24. The method according to claim 23, wherein the second therapeutic agent is selected from the group consisting of ado-trastuzumab emtansine, fam-trastuzumab deruxtecan, trastuzumab, trastuzumab/hyaluronidase, lapatinib, margetuximab, neratinib, pertuzumab, pertuzumab/trastuzumab/hyaluronidase, tucatinib, tamoxifen, anastrozole, letrozole, doxorubicin, epirubicin, paclitaxel, radiation therapy, and combinations thereof.


SEQUENCE LISTING (19 sequences)

SEQ ID NO: 1 - DNA, 20 bp, synthetic construct
  source 1..20: mol_type = other DNA
caggtgctggtggtcaatgg

SEQ ID NO: 2 - DNA, 20 bp, synthetic construct
  source 1..20: mol_type = other DNA
tcaccctagttatagcaaga

SEQ ID NO: 3 - DNA, 20 bp, synthetic construct
  source 1..20: mol_type = other DNA
cagtctgttgtacaccgtgt

SEQ ID NO: 4 - DNA, 20 bp, synthetic construct
  source 1..20: mol_type = other DNA
gactgtccaagtgattgtcc

SEQ ID NO: 5 - DNA, 21 bp, synthetic construct
  source 1..21: mol_type = other DNA
tgtgcttcgagatgtgtggtt

SEQ ID NO: 6 - DNA, 23 bp, synthetic construct
  source 1..23: mol_type = other DNA
gtcaaatagctgactcttggcaa

SEQ ID NO: 7 - DNA, 20 bp, synthetic construct
  source 1..20: mol_type = other DNA
tccctgccagtcctgagacc

SEQ ID NO: 8 - DNA, 21 bp, synthetic construct
  source 1..21: mol_type = other DNA
gttgtgagcgatgagcatgta

SEQ ID NO: 9 - DNA, 20 bp, synthetic construct
  source 1..20: mol_type = other DNA
ggctgtattcccctccatcg

SEQ ID NO: 10 - DNA, 22 bp, synthetic construct
  source 1..22: mol_type = other DNA
ccagttggtaacaatgccatgt

SEQ ID NO: 11 - DNA, 24 bp, synthetic construct
  source 1..24: mol_type = other DNA
tgctggacattgacgagacagagt

SEQ ID NO: 12 - DNA, 24 bp, synthetic construct
  source 1..24: mol_type = other DNA
agctcccacacagtcacaccataa

SEQ ID NO: 13 - DNA, 22 bp, synthetic construct
  source 1..22: mol_type = other DNA
ctcctcctgttcgacagtcagc

SEQ ID NO: 14 - DNA, 21 bp, synthetic construct
  source 1..21: mol_type = other DNA
cccaatacgaccaaatccgtt

SEQ ID NO: 15 - DNA, 57 bp, synthetic construct
  source 1..57: mol_type = other DNA
ccggccaaggattattcgaccatttctcgagaaatggtcgaataatccttggttttt

SEQ ID NO: 16 - DNA, 21 bp, synthetic construct
  source 1..21: mol_type = other DNA
gctgaatttcagtgcttagaa

SEQ ID NO: 17 - DNA, 21 bp, synthetic construct
  source 1..21: mol_type = other DNA
ccaactttaacacagtcttaa

SEQ ID NO: 18 - DNA, 91978 bp, Homo sapiens
  source 1..91978: mol_type = genomic DNA
ggagtcgacaataacaaaccaagccgcggcggtgtccgcggccctgccgagccctcggcgttgcctcagaatcccccagtcgcctgggcccctcggctctgacaggccgcggccttctgtcccccggccccagacccagagccgaggggcctgctcgcgtccttgtccgcccggacccctccctgcctcctagagttcggggccgcggcgggcgggcgcccgggacgccggcggttgtgtcggcttagcggtgccgaatgggcggttggtaaccgctgccgaggactaggcggcggcggaagatggtgccgggggtcgctggctctgctgctgccgccggcgaaggaggaggcgttgccgggtaagtgtcgccgccgcccgcgtccccttcccccatctggcacagcggtcgcgcggcgggccctggcacggtgggccggggtcgcatggtccagggttccggtcggcctgagcccggcccacgcgcaggtgtgtgcacgggagggccccggcgcctcccgccccggcaggtgttgcccactggccgctttcccggcgcgacagcccctgtggagacaaaagcggttcgagtcgatgctccactaggctgccggaggtgtgggtcggactcgtgctgcggggctgcctgcggcttcctcggccggagccttgggatccctccctctgcgctgccgcttagtacccggcctagttgccggagtggccgagggcgagggacgtccgaacctcccaagacgcatcttgacttgtgctcatcgctgccagggatcagcccttaattctgtgttggactcgagaaacgggagagtggagtttggccgaggccgaacgaagctcaccgaatgggaggaacggagagatgatgtctttgtcccgaccccagagtgcgaggatcggtataaagttactcctaggaatgactcagcctgcgcccgccccacggatagatttgggatgggtgtgcacgcggatttcacagcttccttggtagaaacaaaaatacattaaccaacagtagaaatagcagcttctgaatggaatatctctatagtttcttccaccgcctggtggatgtgctgtgtttactgtttatggtttcgtaatctagtcgtttccatcatgactctttaaagtgtggtcataccttgaagtagtgtagacttggctgccaaggaagactcatgtgctcactgttgataaagagtcacgttactacgagacattcagccagatgggaatgttctcaagtctgctgcaaacatttaaagtgtacctatttgctaactattttattgtgtagtttcttaagtttttgtgttttggtgaagcttgtatgatttatacataagtttggacagttagcaatttaggaagagacttaagtagacttaattgtgtgtttaatcaagaatgttattacattgtgatagacgataaaaagagggtccttccacttctgatcagataaaactgtgagaaaccaaggaactactgagggaggtggctgattttagtttttggattattggatttataagcagaccattgctaggaatgtgaaaatatgttcgctttttatatttgttgtaacagaatatggccctaatgaagatattttctcaataacattcctgtcatgtcttttaatgtttgtgattgtgttcaaaacctggataaagttttatgtcagggatgctttaggcacataatgcttacgttgagaaacctctgttgatggaaagtggatattgacatagttcatatttttaaagtaattttatttagtctattttgaactttttatggtaatcaagatatgattatttatgtacctaatcctttatatgtttgttattggacgacagggaagacaatgaagcggtacttttgtccctgagttttttttttttttttttttggctcttgctgccaaacacaagatcaggatgcatggcagatttactttagcaaatcaagttcataaaggcagaagacacaagacttttgttttcgaaattttgaacctaaattttgattatgttagcctgagaaaagtttttagactctaatattaacttaaaattttatatcatacattcagcagatatttgagcacctacatgctttactttgcagatgtgtatgtcttatgaatatctttttaaaagaatggctgataaaattttctcattcaggtaatgatattttctgactccagttacacttttcttttgtagtaatactggcctaagacactttgaaaatctagcagtccctttcaacacactttcacgattcttttattatctttttaccactgtgaggtttttgaactagttacatgaggaacataaaaatgtttaagtaattgttcttgagtagagacggggttttaccatgttggccaggctggtcttgaactcctgacctcaggtgatctgcctgcctcggactcccaaagcccggccactgattgccttttgaataaaggcccaagagaagtaaggggtgtaagtgtgtgactctcgaaaggaatttttggcagaagaaacagtaaatggaaaggctctgatatacatggcatataccaagaaaaatgaagttactgaagctggactggtatgagagaggcgatagtaatggtagattgtggggctctacatattcaggtttggaagtcatagtaaggaaactaatgtgctctaactcacaaaggtctacagatgaattcaagtaaacccagcatggctggaaagcagttgaagccaaattcacttcatttattcctgaaatgctaattgaacccctgcagcatactcttttagcttcagcaatgcaacagtgaacaagaaagacatatctgtctatgtatatatatttatttataggaaatgggtaaccactggaaattttcatggaggtaaatgacaaacgcagattactttgaggatgtagagtagataaaaggctagctaagccgattgtatagtgctaggttgatgagtttttgcttaactcacatagataaaaggatgtgaagattacttgtctctgattatttattatggaccaaatcaacttaatttgactgtaaactgtacccccattgtttagggtcctgagaggatggcatttctttctatccctttgtaaagttccggttctgttggattactttttgtaagtgcagtacttgaatgcctccaagtaccctctttccttggctaatttgtttctgctttcatggctgtttagtgagtgctttgaggattgtggaataacaatcctcgcccattttttttcatccatgatttttttctacccgttagtttgctcttttgaactctcatattttattggcttgtaattgtgctaggagataaaaataattgtgttccatttcctttttttgtcactgatttgagactgtccactcatttttccttcacattccacacattatacttgttcagtaactgtccagcatacctgttgaaatattaattcaataccatattatattacagtaatataacggcttttagtaattgttttactgcaaataccagatttgggcaggctggtttaataatttggttatatgtttatagataatcttggtttaatagaatgcagctaattctacattttcttttttttccccattttccctgtcttatataattctatatttgctatatagataattttggttttactgttagtttccgttataaaataaaacacaactagaaaattttcactaaaatcatgcattttatgtagtttttataaacgatagggacaccaaaatgtgtaatgcatttatatgtactgatacagaaagatttctgttattttatgcagttcaagcaaagtccatagtagtatgtagggtgtcacctcatttttataaaaccatattgcctgaatatacacatatttcctgtacacctagggaaaaaaacctgaagggtgtactccaaagtatttttcatttttgttttaaaagtattcctgttctgctatttttacctcatttgctgtctttgggcactacctttctaaatgctcgcttcattcttttggcgaagttgcttgtctagtttgtagacaaggcgtatatttaggaagtctgaacttttatgaaaaaggttttctttcagggaaatgtgaggaaaatgcctgacctactcatctttcattctttttcttggcagagaaggggtaggatcagattagaatacatctgatttggggagactgatgcccagttttcagtccctgggcaacctagacactccgtttgtggtggacattatctccattgatgcagtagcccgtgacccctcaatgagggagaattgctaataattgcagtttacagaattagagatcaggcaaaattatttccaatcatcttttctctgaagcttttagtccactttagggttatctgagtctcatgtgttctattcagctggaaagcatcttgaagatatttgtattccaggcacctagcagtgtctggcttatgtttagatgcccccaaaatagtggatgaataatgaatgtgtccagttttgaaggtcagagattgtcattggggtgtccccctttcaccttatcatataaggaagcgaattctgttttgtttgtttgtttttgttttttaaggcagagtcttactttgtcgcccaggcaggcgacaggcaatggtgtgatctaggctcactacaaccactgcctcgtgggctcagatgatcctcccacttcagcctcccagtaggtgggattacaggcatgcactaccactgccaactaattcttgtattcttagtagagacggggtttcaccatgtttcccaggctggtcttgaactcctggcctgaagtgagccacccacctcggcctcccaaagtgctgggattacaggcaggagccactgtgtctggcctaattctaagttttgactgtattagaatagttgacagtaaggctgggataattgactttatcatcatgataatctctcgagaaaattgtaaatctccacttgataagttggatagattttaatgagtatgtgagatactttctggttcttttcctcaaatatggatatatatgggttcagttgttattcctgtacatctggaaacaaatgcagtgggatcaatatatttttatttttatttatttttttgagatggagtttcactcttgttgcccaggctggagtgcaatggcatgatctcagctgaccgcaacctctgactcccgggttcaagcgagtctcctgcctcagcctcctgagtagctgggattacaggcatgtgctaccacactcagataattttgtgtttttagtagaaatggggtttatccatgttggtcaggctggtctcaaactcgcgacctcaggtgatctgtccgcctctgcctcccaaagtgctgggtggcatataaagtggactaaaagcttcagagagaaaagatgattagaaataattttgcctgatccctagttctgtaaactgcagttattagcaattctccctcattgaggggtcacgggctactaccgcatcaatggagataatgtccaccaccctgtcggatcaatattttttaaaatacagctctcttctccccttcatttatagctgactgtatcctaggaggatttaggaggaccagatgtcctcataatgagagataggtttttccccccagataagagaattcctgctaccttatattaactacttaagagttctgtaaattcagagaaaggagaaagaatactataggccacctgtgataagggaagacttcccagaagtggtaataggacaatacttggacttttgtttgtttgtttgtttgtttgtttttgagacgagtctcgctctgttgcccaggctggagtgcagtggtgtgatctcaacttactgcaagctccgcctcctgggttcacgccattctcccacctcagcctcccaagcagctgggactacaggcatccgccaccatgcccggctaatttttttgtatttttagcagagacggggtttcactgtgttagccaggatggtctcgatctccttaccttgtgatccacccgcctcagcctcccaaagtgctgggattacaggcgtgagccagtgtgcccggcaatactttgacttttaaagatgagtaaaattcagatcagcaaagatgggggtattttacctggcaggagtatgtaaagtgagggtataggtttgtcatgtaagggagtagttgatgatactgctacaaaaataaactggggttaattatgaagttcttgagtgctggagtttaattaaagtcataattgccaccatttttaaagcttttagtatattcctagcaccgtaaaggactctgaagtacattattgctatccttctcatctctgtgaaaggtatcgttgcaatttatagattgtcaactgatatgtggaggttacctgagcctcagtgagatagatggtattcaaatacaagtaaattttgttcctaagtctgccatttttatgttaatctagcctatatcaagttggcaaattcacatgctaagaggggtgggcagatagtacaattaagtgaaactgtctgagtactgaaccggaggtgacaaagagagtgtagctgtaaagaaactacagatttgtgccctgctgtaggcaacagtaaggtgctagggaagatacttgagggaagcagcctgactaccttggttgtaactggtagggagtatttaggaaggattgaagccggatagagatttaaagacagagagaagaggttgcagtttcctaagtaagggttatgtcattggaaaatgaggggttggattagaaaggaaaaaaatgggtaggacttggacagaaataactgaagcctggtccattgtcactttggaaagtttgtaaaaaaaaaaaaaaaaaaagaaagaaagaaaaagaaaagaaaaataactgaagtttggggcaatagtagaatgatagttcattgacagtgaataaattagggaatagatttaaaggaaaagcaccgaatttaaatttggacatgttgagtttgaggtaacagtaggatttccagctggaaatatgaagcatcagttactgctagctctaaactcagaacagagatctggattagttaataacagtaattgatgtttgtatttcactttatggtttacataacttccatatatgtatagattattcgttagatcctgaccacaaccctgtgagacaggtacttttactttttgtaatcaaatgaaaaaacttgttaggtgaagtcagttcaggtcacattgggccaggacttgaatatacttcttttgattccaaatctcatgttcttttcacagttctgttctccctcttatgtttacataaagataacaattggggcgtggtgactcatgcctgtattcccagaactttgggaggctgaggcagacagatcacttggggtcaggagtttgagaccagcctggccagcatggcgaaaccctgtctctactaaaaatacaaaaaaaattagctgggtgtggtggtgggtgcctataatcccagctactctggaggctgaggagagagaatctctctctctcttttttttttttttttgaaacagagggagtctcgttctgtagcccaggctagagtgcagtggcgagatctcggcttactgcaagctccgcctcctgggttcacgccattctcctgcctcagcctcccgagtagctgggactacaggtgcctgccaccacgcccggctaattttttgtatttttagtagagacggggtttcactgtgttagccaggatggtctcaatctcctgacctcatgatccgcctgcctcggcctcccaaaggagaatctcttgaacctgggaggcggaggctacaacgagctgaaggctgcagtgagcggagatcgcaccactgcacccagcccctaggtgactgagtgaaagtccatctcaaaaaaaaaaaaaaagataacagttggaagactaaacatgaatgaccatttagggaaggctatatttagaatgggagtggggcagaactcccctaggacctcagattgggactgaaatagggaaggctggtgggaatctttgtcttgctctcttggtgggactaactcttttggtgcaacaaattcaggttctttatttctttgtaatgtatagggtgtcatcttttttttttaatggatatgtgtttttcccctctaattataaatgtgatttttaaaagaactgggagaaagtaaaagtctggcatccagagatagccagtgttaaaaagattgctattaaattgatccagactttttaagtgcttgcatatacatatgtcctcctgcattagttgtaaaaataaaatccacaaacagaatcacataatatatactgtaaattgctttttgtactgatcaatacgtatagatcaaaacatttttaaatgtctaaaatttgataccttatagggttgtgtaataatttattatttttttggagagaggatcttgctgggtcacccagcctggagtgtagtgatgccatcatagctcactgtagcctccagctcccaggctcaagcaagccatatcctcctgcctcaaccttctgagtagctaggactataggcataatttatttaaccaacctccattgatagtttttaaattcatcattttaaaaagtgatggtataatgaaaatcattgcatatatatacctttgtacacttgtttcattctttgctcccagaagtagaattgctggtttgtacattttaaattttgatacatagtgccagattaaattcccttaaaagttataccagtttatatttttgtcagaaatgtgccagtggacccactgttaatcttgtcaacacttagcactcattttaatcattagtagtaatctgaagggtaaaaagtagtaccttgttgatgttttagttctcattagtgaatatgtacctccagatagtttatttttcttcatgctatggatagccatttgtcataataccattgcatgaaaagttgatcctttctccccaatttgaaatcccaccattattgtatagatacacaagtgcaggtatgcatgtataaagatcaatttggatctgttcagtttcatgcattatgtttattaatgcatcatgattcctactaacttgttaactaccgtgtgagttatgagtatgtttgacatgttttgacagttgttctgtcctacgaagtctcagtatatatggtaggcagtttatagtaattcttagtgggtagaaactaaagactgtttaagcagagggtcaaataatttaagaaaatcttgtgtcagttgttgagttactaacctgttacttttaatttagaattggcttgtgttaacttgtatagtattaaatatggtttcattttataaaagttaagaaggactttatataaaatgagcatcaagtcagattttaggaaatcgtgactatttggtgaaaagtaatacttatgtactaggcttatttatataaggcttataaagttactttataattccttgaatagcagttgatttctgtgcttgtaccttacttaacaattacatgcaataactttagttctagatcatagagagtcaacccacttacaggatttaatggactttacgtaaattaggggtacatgtacataataagcattcccaaagttgcaagttaataaaagaattagctccttttaatgattgttttcttaaaagaaatcattggtttattaagtttatcctcttaagttttagtaatgtgatcagtgagggtaaactgtcagtttctagttggaacttgattttacatatacttttgtgtttttttttttttttttttaatgtcacctttgtgtgaattcttgtccattctttctcagtctaacttttgcatacctctcttcatattagtattattgaactttttggaaggcaggtaatgtgctttttattactttttggcactaacacacaataagtttcttaaaattagtatttgttcagtgtgttagactcatgtttaatgtatgatttgactatttatagaagatcagaagaataatctagtgtacccctttatgttttaagatgacgaaactgagattcagagaaattggataactcctgaaatcacaggagtaaagatgtgaagctaggactaaaaaccagatctcttatttctttccaagtattttttatttcagggtgaccaaatatgtacttggttactgatgtccctttttgctgaatgttttttaacaattgtggcatacaaaggagagaattcagtttcatgggatcatgttattttgatagcatataggagtatgtaacataaagtatacagtcttgtgaataaatagcgacagtgttcctgtggattctctctgagcaactttctgatttcagttttggttgccgtagtccttatccatggttttgaccgcaaacatttattgactgtttactgtctctaggtagtatgttaggtattgaggtatgatatgtcatgaccaaaattttttttagccatttgcaatacagcttgggcgatgtaagttatttggcaagtggcaaaaaagtgtagcagggcatttatttatgttctgaataccaacacttacttgaatagttggaatttgaagtatgggctgtcagtacttcaggaacaagaccactgacagccagtacattgaggaagactttacgacagaatatttgtgaattcttggttacataatggaagctgtggaatgacatgagtagaggcatgaaaatccatttattcatgcacctcatttcaggagatcagcctaaatggtatggagcttcatgtagggacccaaataaaggaaatagtggagtgtcagtctaaggaatttatacttgagtagaagttatgaagcatttttagtttggggagaagtaaacattaaaagaatctaagattatgtagttggtggtgtcatctgagatggaatctgcagatagtaggtgctcagataggtaattgttagagcccgatatgcagtaggaagatctgaattagatagcttaatgggaataaggtggaagagaggatacacatacttagtaactgtttatgtggaaccaaggagtacttattcatatatgatgttaaaatatattaaaccaagttgattacttttttttttttaatataagatactgggaacacattggctgataaagaaactgctgaaaattagctacctgcatgtgatctctctctgttactaagttactatttggattctgctttgtcatttgcttagagtactgctactagaaccccaagagtacatttgcttatgaaaccctgtttattcaacagatgcttttgagtgctttgtgttatgtcacagagttacttcctgtccctagagaacattttgggaatatttactgtgatttcttttttttttctgcaacagccatatgaggtaggtatgataaatatccccatttacagattagagaatggaaatttagacagacatgtattgatgaagaaagttgttttcttgttttatctacatttttttcttcttcttttctttttttttgagtctcactctgccaaccagctggaatgcagtggcatgctctcggctcactgcaacctccacctgccgggtgcgagtgatcctcctgcctcagcctcctgagtagctgggattacaggcgagagccagcatgcccagctaacttttgtattttcagtagagatggggtttcaccatgttggccaggcagtctcgaactcctgacctcaagtgatccacctgcctcggcctcccaaagtgctggaattacaggcatgaaccactgtgcctggcctgttttatctacatttctttccctgataataaagccatgcagctcatggatcttctggagaagattcattcagtagaattttataactttgacttagattacacaaaattggtctatgtaggtcatatgatacttcttttaataagagaacttaaaacattagcatgaataggtatattttattcatcctggtacccatacagagtatttaactaattttataaaccatacatttcaattaaaatctgaccagctttattacatgaccacataaagattaataatccagggttgaaataacagctaaagatagaaatagatagttttggctgtgcaaggtggctcacgcctgtaatcctagcactttgggaggccgaggtgggtggatcacctaaggtcaagagttcgagaccagcctggccaacattgtgaaacttcatctctactaaaaatacaaaaatgagctggacgtggtggcaggtgcctgtaatcccagctacttgggaggctgaggcagggagaattgcttgaacccgggaggcggaggttgcagtgagctgagattgtgccactgcactccagcctgggtgacagagtgagactccatctcaaaagaaaaaaaaaagtgatttttttttttttcaaagaagactttagtggtataggagctaaggtaaagaagagaaaatttgagatcaagcagctttatcaagagatttaagtgacagaaaagtgatgatctggccgagcatggtgagtcacacctgtaatcccagcacttggagaagccaaggtgggaggatcatttgagggctggaattcatgaccagcctgagcaacatagagagaccccatctcttaaagaaaaagaaaagaagaaaaggaagtaatgatccttgtgagcaaaaacaggtcagtatcctcttgttttgactggatcagagtgttcaggcaaagcagtaatcaaactcctttttccatttactgctgttgtatagatctggtcccttggccagtcacagtggctcaagcctgtaatctcaacacttggggaggccgagacaggtgaattacctgaggtcaggagtttgagaccagcctggccaacgtgctgaaaccctgtctctactaaaaaatacaaaaaattagctgggtgtggtggtgggtgcgtgtaatcaaagctgctcgggaggctgaagcaggagaattccttgaacccaggaggcggaggttgcagtgagctgagatcacgccattgcactctagcctgggcaacaagagcaaaattccatctcaaaaaaaaaaaaagatctggtccttcacacattgttatatacataaggttgtacagccatttgagaaagagaccagttatgtctgtttccctaaacaactggcttgagagtccaatttatacaacttacttgtagaatctttagtaattaggcactttgggtgtcacctaggtcaacctccctcttaatgcagtgacaggtccagggaaattcaattcaagtaaagatttcacgtttcatgcctgcagtggttgttgttaaacattgagattgagagtgtagtctgagaataagaaaaccttgaatatcctttatattggttagatgttggtgcaatttgtttttgtgtatacatgtttacttataggaaactgtaattttcattattggcaattgtttctatgttccctttatttagtaaagcatatttagtgtttatgccttgccatggatacagggtggtcatatcagacattgtttgtatgttctgctcatgttaatggattggctgcaaataagatatgccaatgaccaataagatatgcttagtccacacttaattaagaacatgttgctgattttttttcttttctttttgttttttctaaagatagtcttgctctgttgcctaggctggagtgctgtggcacagtctctgcttgctgcagcctcaacctcccaggttcaagccatcctcctgcctcagcgcccccaccccacccccccataactgggactacaggtgtgcgtcaccacacctggataatttttgtatatttgtagatatggagtctcaccatgttgcccaggttggtctggaactcttgatctcaagcaatccacctgccttggcctcccaagggtgctgccaatgcacccagcatgttgctgattttctttgagacatagtggcaaagaaaattttctcagctctgtcttaccaccgtgaaagactgccttcagagtgtttcagttacctcttcccatcaatatgagaaaagatgagtgtgaggaataggtagagggaaaaattaaagttttttgtgtagcatgaattatgggaggggattatataggtaattctagcatgcacatagctcttgtataggcattgtgccagtaggtaggtattagagtagacatggttctttgtagtagccttttataaaattgacttgctttggtgaaacagctcctggcatcattcagacatttttatcctgttgtgtgggtgtggaaacactgggattgttctataagctttcttttgtttttgtgtgtatatatatatatatatatatatatatatttttttttttttttttttttttttttgagacggagattcactgcagtgcagtggcagaatctcggctcactgcaacctctgcctcccgggttcaagtgattctcttgcctcagcctctcaggtagctgggattacaggcatgtgccaccatgcccagctaattttgtatttttttttttttttcactagagacagggtttcaccatgttggccaggctggtcttgaactcctgacctcaagtgatccacctgcctttcccttccaaagtgctgggattacaggcgtgagccaccatgcccggctgtatttttataattcttaatggaaatcaaaaacacagaaagataggcaatagaagaatgagctatgatgtacccatcattttgattcagttatcatcacgtcatggccaatcttgttttagctatacttccatctgcttctcattctcttgttacttttgacctgtaaattttgaagaacagatatcccctacagtagccctgtgaggaagatgttatctctacttttgagatgagaaaattgaatgttaggataaatttcttttctaaaatcacccagcttgtaagtggcagaataagaattttaaccaggtctgttaagttcttcctctgcagtcagttgtttttccttctgtttaggtcagattataaccaaggagggtggtgtattctgacagcagctaaatgttttactggagtattaccaattcagtacctacagacacttgttaattgttttgggttagggttagcttagagttagatttttttcctttcctctgcattatttttgaccatttaaaaaatcatattgaaggccgggtgcgctggttcatgcctgtaatcccagcactttgggaggctgaggcagacagatcacctgaggtgaggagttccagaccagcctagccaacatggcgaaaccctgccttactaaaaatacaaaacaattagccgggcgtggtggcaggcgcctgtagtcccagctactccgaggctgaggcaggagaatcacttgaattcgggaaggggaggttgcagttcactccagcctgggcgacagaccgagactctgtctcaaaaaaaaaaaaaaaaaaaaaaaaaaaaaaaaaaaaaaaaaaaaaaaaaaaaaaaaaccaaccattacatatacagtttttgttaatactgtaagccttattaaaagttattctctggccaggcgcagtggctcacacctgtaatcccagcactttgggaggctgaggcaggcggatcacgaggtcggagatcaagaccaccctggctaacacagtgaaaccccgtctctactaaaacatacaaaaaattggctgggcgtggtggtagtcccagctactcgggaggctgaggcgagagaatggcgtgaacctgggaggcggagcttccggtgagccaagatggcgccactgtactccagcctgggcgacagagcgagactccgtgtaaaaaaaaaaaagttattctctcatattttaaaatttctattcaagacaaccctgggcaacatggagaaacgccgtctctacaaaaaaatacaaaaaattagctgggcatggtagtgcatgcccatagtcccagttacttgagaggcttaagtgggaggattgcttgagccggggaggttgtgaggttgctgtgagctgtgatcccgccactatactctagcctgggtgacaggacgagatcttgtcttaaaaaatgtataaataaatcaaatttttaatttttatccgtaacattttaaatcctgtcttaaaatatgaataataaaagtatgggtagacaaatatttgttactaataaatgtaggaaagtgtcttgtgtatgctttaactatatttcaaattttttccctaaaattgatttcatggtacagttattggcttaaggaaatgtgattttataaaacctttgcttacctacctctacaaacaccagttgctttccagaaacatcataattatttaaaatacattctgacctgtaatatgaatgttgaatgtgattaaaataatacattttctagtgttttattatggaaatttgcaaacatatacaaaagtagagagaatgttacagtgaacctaatatagccatctcacttgctttaataagtaccaacattttgccaatctcacctttctactgcccagctaattctgttctttccttccagccttccttccttcgcttatttttgctagactagtttaaagcaagactcaggcatcattttacacactaatttttcagagttcattttctgttactacaatgccattatcatccaaacaaaagtaattctaattctttagtataatccgataccctgtccgtatttagatttgcacatttatcccaaaaatgttattatacagttgttttaagtcaggatccaggatctgaacaaattcacacatggcatttgtttattttgtctcgtccatcttttttagtttataaaaagtcttcttttttttttttttttttagtaaccatttgttgaagaaacagagtcattgttctgtggaatgcttattctgaatttaactgattcatcataatgtttgtctttttctttatactccatatttcctagaaactggaaaccgtaggtacatatagttgcttgattaaattcaggtttactttttcagaggtggtctggggtggcaagaaaactttatacttgggctgtgtccagttgaatcgctattagctggaggttttccatataaatggactgaaatacgtatcttttttgttttgttttgttttttgttttgagacggtctctctcccttgcccaggctggagtacagtggtgtgatcttggctcactgcatcctccatcccccaggctgaaacaatcctcccacctcagccccgcaagtagctgggagtacaggtgtgcaccatcatgcctggctaatttttgtatattttgtagagacagggttttgccatgttacccaggctggtcttgaactcctgagctcaagtgatcagcctgcctaggcctcccaaagtgctagaattacaggcatgagccactgtgcccagctatccaaaatacatttcatataggaaggacagtgcttaatttctttgctttttaagtttttatcatgggttctatcttagctcactgaggttttttgtttttgtttttttgagtatcattatgcagcatggatttttatgggtttttgccattcagttacttgtactcattcttttgatggtcagactgtcccatcatagccagtagaagccttttaaagttgttttccttgcctttttgacctaatctctttagtctttgttagctgtcttgctttatggcatattaagatgttccaggcttatgtgattggtacattttctactttagacatttagcattagccacttctccaaaggttctttctagaaaccaaagtaggccgggcgtggtggctcacacctgtaatcccagcactttgggaggccgaggcgggtggatcatgaggtcaggagttcgagaccatcctggccaacatggagaaactctgtctctctgaaaatacaaaaattagccgggcgtggtggtgggtgcctgtaatcccagctacttgggaggctgaggcaggagaatcgcttgaacccaggagacagaggttgcagtgagccgagattgtgccattgcactccagtctgggcgacaagagcaagactccgtctgaaaaacaaaaaaaccatagtatgagtgtgtaatgaggttagccaactctacagttagaaggaacagtcccacaaaactgccctcatttcaggtactagtggcaagtttggcggtgcccagaacccaccctcacttcaaaccagctgactacaaactcacaggttccccatatccaccctctctttggagaattcactggaacaggtcagggaacgaaggaaagcactatactcacaattacacttttattataagagaaggatacaaaaattaaaatcaggcaatggcagagagcagaatctggaaggggtccaaactcagagcttctggccatcattgctggtggagtcatagacagtgttaccttctcttggccacaacgcgtgacagtatacacagagtattgctcactagggaagctcacctgagtctttggtgtccagagtttttattatggcttgatcctgtactgtccacatgactcacctttagtctctagccctttctggaggtcagaccaatgtctttagtcagtagttcctctggaggtcagaacttaactgatacatgagtcacgttgctacactgacgtgactgaagccctaaggcaaacaaacacagttctatctgtcaggatattacaggggcctagagatcacctcccagtagcgaagggcaaaggccagacctttctttggttaatgttaatttttcactgcctgttgtgctgtgggtacttctggctctaggtctgtcaatgccttcttttcaatggagctatggaataaatgttttatggaaagagaaaaattcttgtttatactagtatttctaaagtactttttacttctttatacattttatttcttttctcttgggttgaaaaccttggaatcttaggtcgaaaatcttgcttgcttagtaggagaaaaatactgtcttctttttagatttgtaattctttgattactgaagaggcctcaaatattttccacatttgttaaccacttgataacctactcatgccctttgcctatttatctattgtgagtctttgtgattttcttattaatttataggaactcctactaaaaagctaatagtaatccttagtcacttgttattgtcattttcaccagtaatcacatctttggatatttgaattaaatgatgggtggcaggtatggaaaatatcttcttctttttggttttctaccctttcctctcccactaaactagaataccagtcattgtgtgatcagtcctgggtctatcatctgttagaaagaaaactgggaaattgggcttgtctctgggcaggacttctttgtattgttgctgtcacttccatatgatggaagtgctgcttaccaggaggttcagtccttcactagtagatgcctggtattaactaaagtatgtgcagctctcactgaagtggctgtagttgcttaagctttggaggaagaggcacacactttctctctaaaggaccagatagttaagtattttaggctttactctccaagaggcaaaattgaggctatgatgtatagatgtttaatgtaataagagagaaaactcctgctgcgtcccgtttgtccgggatgagccatccaggatggtgggcatgttttgtgcccagttggcatcagttcgagactgtctccggatccacaggggactacccgggggggatgagcctgggagtagaggccaacaccatgttattttctgctttgagagcagatcctgaatcaccctcctcttcttcctgaaagcctggtgggagaccacgtgacctgttgagtcttccactctagagccaagattatactcctcactgtcctgcagttgccagcagtagggtggagttccctatttgaaggtggccccagtgtgtgtcctgggagtgaagtgtgtgaggatgcaaaggggtagctgtctgtgttaaacactcagctctgttcttggctacgataggtaggttctcctctatatgtttctttagctgtgaagtgttcagtatgcacatttctagagaattccacactgccaggatgaccaggtatggaaaaatttctctctgctacatgccaagatcaccttttcttttcttaaattttaagttaaagggtacatgtgcgggatatatgggtttgttacataggtaaacatgtgccatggtggtttgctgcacagattatcccatcacctaagtattaagcccagcatccattagctattcttcctgatgctgtccctcctcccacccttcaccctctgacaggccccagtgagtgttgtttccccatcctacccccgtgtgtctatatgttctcatcagtaacaggattgctgggttaaatggtatttctgcctcaaggtctttgaggaatcgccacactgtcttccacaatggttgaactaatttacactcccaccaacagtgtaaaagcattcctttttctccacaaccttgccagcatctgtctttttctttgactttttaataatatccattctgactggtgtaagatgatatatcattatggttttgctttacatttctctaatgatctttgatgttgagcttttttttcatgtttgttggctacatgtatgttttctttttcttgtaaatttgtttaagttccttgaagatgctggatattagacctttgtcagatggatagattgcaaaaattttcttccatcccgtaggttgtctgttcactctgatgatagtttgttttgctgtgcagaagctctttagtttaattagattccatttgtcaatttttgcttttgttgcaattgcttttggcttttttgtcatgaaacctttgctgtgcctatgtcctgaatggtattgcctaggtattcttctagggtttcatagttttgggttttacatttaagtctttaatccatcttgagttgatttttgtatgtggtgtaaggaagggattccgtttcagttttctgcatatggctagccaagactccaagcaccgtttattaaatagggaatcctttccccattgcttgtttctgtcaggtttgtggaagatcaggtggttgtaggtgaagatcaccttttcatttatggacacaaggaaatcttgttctctcaggatctgggtccatgccatttcttggatgatgactttgctttctgttgaaaaccttaccttgtcacatttaaagtgttctgttattccactcttcttacttcatattgaaagaggttttttttgtattgtatttagtaaaggaaattttttaaatggtcacataagggagaaaatctgcaaggggaaaaaagtgttgctatttctaaaggaatagtaaagtcccaagtcatagaatatgcttactacatgtagtcttatttaagggattttaatgtagtatgttcatgttcttttttcccccgtgatccagctatggattttaagtttttacaactttctttctatgtgactttaggaaataacttctttgagccatagttttgtcatatgaatgatgcggataattcttaacctctctcactataggatttgtaagaatatcatatacttcatacagttcttactcatgtttccagtcaatggaagctattgctatgccaacagtatcattatctctaaagaataaaagcaaagtggaagtatgtattttggaacttcatgtttgtttttactttaaaaaaaagattttttgtagcttttttccccctgaaagtaggtatcatggagttttatataaatataaaattttcatgttgcccactatgatattaaaattcatgagagcagacagttttattattttgtattttattcccaaagttaagaaaatgcctggtacctaactagattctcaggtgatatttttattgaatgaatgaatttattgacctgtgttcacacccctggctaagtttacttttgttttggtttactaagtttgttcctttagacaagtttaatctgaattgtaatttctcatctgcaaatggaaaccctaatgatatgaatgtattacatgtaatgataaatgaagtcatgcatgtaaagctacttagcattatgagtggtatacaatgaataactcaataagtattagcaatgattatgattatgattttatgtgccatttacttaggacatgtggaggaaagaagagcatttgtggccgtgaaagcaatgagcgtcacatttcctcacaggttaccggtactggtagacccatgggaaatgatgccttgtgttagtagttggatttttctctgttgttcttgggtagacttttggtagctggcagttagtgaggatgtagagagtgaaaatatttagaagaggcttctgattaaagatgccagtcttaggactatacacaatggatgaaaacgtgggaaaatgatattgtcattaacatcacttaggagctgttagaaatttgtatactggagtcaaattttaggttctagagtcacagttatcaaaactatccttgaagtggtttaaattaattttaacctaggttctggagccagtgtaatcaaaactatccttaaaatagtttaaattgttcatcaagtatgtagacaaactcattctcaattaggtttatttctttcagctttttttggtcagcattgtttatgattatggtttcttagggtgaggtgtgtgcatgtcaaatgaagagattagtgtttgtttaaaggcacacagctgctacagcttattggagtttccctcaatttgctttaagtatcatggaagaggagaatggaactgaggtcgtctatactctaacgatttctaaagcttctgattgaaagcaatatattggctgtagatcatgcagaggaaaagctgaactgtcccagattggaagagattaaatagatattacagctaaaagcaatatgatgatcctgaaataaatcctggatctgaggggcggaaactacaaaggatattattgaggcagtagtttgaatatggactgtagattagataataatagtgcatcagtgttaaattttatgatttgataaatgtactttggttatgaaggagaaaattcttgctgttaggtaagacattctgaagtatttatggataaagggaaattctgtctgcaaattacagtagttcaggaaaaaatatatccctgtgtgtatacgtatgtggagagagagcatgtatggagtgtgcactgacacaagtgataaatagaacacaagtgttcacagttggtgatctgagtaagggggacgggagttctttgtactattcttgtaatttttctctaactggaattatatcaaaacaaaagataatattcaaaggaaaacgagaaaacaaaaaacaatacagtgattattattgaagattttgtgtttagtctcacaaaaatggtgtgtctccttgtactgctttacatcttctcagttcaggcctcttcttatcccaacaataccgtttaaatgtaacccacgtagaggcctcctttgatcacataaatctaaagtaagtcgttcttcctacccactcttttcttttgtgcgttgattacagcttatatcatgtttgctttgttcttgtttaatgctgctttctccttcaagactgaactccatgaggacaaagactatgtctgttttttcccccacagaatacccagtcctggcacataatatcaatacacgtgttttggatttggatgaatgaatgaatgaaggaatattccttccactttagtcagcatcccttagctacctaatctctgagaaaacaaaatgcctcctgtaagataaagctgtaagataaagcctatgcttttggatttggtattcattcactcattcattcagcaaatctttatttaatacagtcatctgattcctgtttatctctagcttccttttgttaacatctctcagatctgcatgacttttctaggtctgcaccttctggccagtcttctgtcaatctgttaagtgcgtttcatagttccctgtcactctccacatatttcttagaaagtatcacatttatgataattatgcctactttccctggctgtagatactgccttttttttttcttttgagaaagagtctggctctgtcgcctaggctggagtgcagtggtacgatcccagctcactgcaacctccgcctcccagttcaagcagttctcttgagtctggaagaagatgtgtatgaggctagggcaggtggaaggggtgatcatagcaggcagggcaaatagaatcctaagcgtggaaaactgccccatgaagactcagagactttagcagctcgatttgtttagggagttcaagcagtgtgatatttgggactgataagttgggggaggggtagaggatgagggtgaaaagttgtggtggaaaggaagagcatattatatttgctgtcttaggtgttattgtcatttaataaagcatttagaatagtttcctggaatataatgaatacaaagtggtagctgctgccctaaaagtcataatcgtcttgagttttaaagcagcagaaaaacttggatcaacctacctagcatgtaggaagatttctttgaaggcagcatggaggttgacggactagtgattgaggaaatcagtgcaggttgaaaacagggtctgatttaaggcagtagggatagaaatagggatatcgtgtcattaagtatttaaatgttagaatttacaggacaaatttgctgatgttcgtatgtcttttaggaggaagacaaatctaggataatatgtgagagattgggtgaatctagacttcatcaacaaagagaaatgaaaaattaaagcaaggagatttaaggggcagagctcagttttagaaacaataatgttgaagaaatgcatataggagattgatttggatttgttgagtttcagagggattggatagatagtttaggagagatttgggagtcagcaatcaacagtggtggtagcagcagctatgggagtaagtgagctctactacaaaggtaaggcggctgagatccctcccaaactttggagagttgacttatctgccaagtaccacattttttctttgacatcctattgggggaacccacccccaatatttcactgtaggttctattttccattagtgtcggccggctgagaaataaagagagacagtataaatagaggaattttacagctgggccgccgggggcgacatcacatatcggtaggacagtgatgcccgcataagcctcaaaaccagcaagtttttattaagggtttcaaaaggggagggggtgtaagaacagggagtaggtacaagatcacatgcttcaaaggacaaaaagcagaactaataagagtctaacaaagatcacatgcttctgagggaacaggacaaaagacaaaagcagaactactgataagggtctatgttcagccatgcatgtattaccttgataaacatcttaaacaacagaaaacagggtttgagagcagagagccagtctgaccacagatttactagggcggagtttttccccaccaccgtagtaagcctgagggtactgcaggagaccagggtgtatctcagtccttatctcaaccgcataagacaggcgttcccagagcagccatttgtagacctccccacaggaatgcattcctttcccagggtattaatattccttgctaggaaaagagtttagcaacatctctcctacttgcacatatgtttataggctctctacaagaagaaaaatatggctctttttgcctgaccctgcaggcagtcagaccttatggttgtcttcccttgttccctaaaaatcgctgttaattctgctctttttcaaggtgcactgattgcatattgttcaaacacacatgttttacaatcaatttgtacagttaacacaattatcacagtggtcctaagatgacgtacatcctcagtttacgaagataacaggattaagagattaaagacaggcataagaaattataaaagtattatttggcaactgataaatgtccatgaaatgttcacaatttatgttcctctgctgtggctccagctggtcgctctgtttggggtccctgacttcccacaacaacatcctcctaatatccctgtgatatggttgtgttatattaatcatctattgttgagtgagaaattacagtaaacttgattgtttgaagcaacataaatctattattttatagattctgggcatggcttcactgggcccttttgggtctctgcaagacttcagtcaaagtgttggccagggctgaggtcttctctgaggctcaactaggaaaggatctgcttatgggcttgtggtggttgtcaagattcagttccttgagaattgtcagactgagactgtagcttcttgctagctggccatcttcaattccttatcaggtgggcctctctatatggcagcttgcttcatcaaagctggcaagagagagtcagctggcaagatgggagttataatcttaagtaacgattacctacatgatatcccatcatttttgcaatatttttttggttagaaacagatcacaggccctgcctacacttgagggtggggatttcactggagtatgaatacagtaggtggatgtggtacagataattgctgctgtcttagaggctgtctgctgcaggtgccatttttattcctatttacaaaagaggaagccaaaacagttgtccaagatcatataattgcaagtaatggaccccaaaccaaacccaagctttctaactccactgcctggatacttctttaccataatattttaaggaatgaggaattgttgtggaaatggccgttttgaggaatttattggtgacttttgtttgatgccatggattgatgtgggttgaagtcatgtggttgttattatagtgaatgtgcaagtagaagggcctgagtagagattattcttagacggtagttttaaaaggaagaaaggaacaatagccatatgttgtttcgtactgaattgtttctctttataaccagaaatttttcttttccagttttctgagtttaaccagtaatgccattcagttgccaatctcaagcaaagcaaacataagccagttttaatctactttttaagaaaagtggtagtccttttcacagtgcctgacggtaagtcacagtttctacctgtatagcagagtttaatctattgcctttaaaataatgattctggaaatcttaattttaaaaccttatttgctttttccttctctaataattctctaaggcttatgtggaaagagcataagtaaaggtacctactactttctgggaatttatttatagttatcgttcaccagtttcattaatcttatggatcttttcctccatttgttgaaagtaaaatgctagaccatgaaatctgtcccctttttatgtcattattttaagttttttttctttctctttttcttttgagatagggcctttctctgttgcccaggctggagtgcagtggcacaatcgtagctcactgcagtcttgaactccttggctcaagccatcctcctgcctcagcctcccaagttgctaggaccacaggcatgcgccaccaagtgtgtctaatttttttaaacattttgtgcagacagggtgttgctgtgttgcccaggctagtcttgaactcctggcctcaagtgatcctcctgccttggcctcccaaagttctgggattgcaggcatgagccactgtggcttgttgtgtatgttatcctcattttatagatgaaaaagctttattttctagatgaggtttttccttgggcatgttaatcacagagttgttttaaatattctgatctccagacctgtacacttgctactgttacacaaagtgatggaactattaaggaggatttcttgctcagtgtagaggtgaaaagcagttcaacaggcagctgatggaagtgatggaactattatggaggatttctagctcagtgtagaggtgaaacacagatgatggaaggaattgggattcatttcccctaatttgcaagtaacggctactcttaatttgcgattagtaagtttaaccttaatactaatctattttttattatgttgaggaagaagtggggggaaaactctcagggataagatgattgaccaactgttttattcttaatgtaaaacactgagtgaaatatgcacatagcaacctttactctagctactggccttttacttcttgaagaatcaaactttttttcctgtggcagaacagttatgcttttcttttcactaaactcattactatttatttttatttttttttaagcattgagtttactgaactatccagttagccatcctagacatcaaagcccattttgatatttttgttatttatatagtaatactgtactgtgtcataatgatgatagtatttgtgattgtagttgcatttgtaaaattgatcacttctcttgtttcttctagtaactgtatcagagggtgaggtataagctcacagaattcagataaatcatcatgaagttatatgtatttctggttaacactggaactactctaacatttgacactgaacttacagtgcaaacgtgagttgtatttcatttatttttttttcgcattctgtctgcatgcagaaagttaaagaaatattattctactgtttaaaaagtaggataatggcggccgggtgcggtacctcacgcctgtaatcccagcactttgggaggctgaggcgggcggatcacgaggtcaagaaagatcgagaccatcctggccaacatggtgaaaccccgttcctactaaaaatacaaaaattagctgggtgtggtggcgcatgcctgtagtcccagttaatcgggaggctgaggcaggagaatcacttgaaccccggggggcagaggttgcagtgagtcaagatcgcaccactgcactccagcccagcgactgagcaggactcttgtctcaaaaaaaaaaaaaaaaaaggtcacataatggcaagtcttaaatcttttatcccaacatattttatatgtaattgttatctttctctaaaagactagttacattctttaaattgtgctttgtcctgttggcatgaatataatattcatgtaaacagtaaacagatcagtaaacagatctactcacacaaaacaaactgatttccattgttgtaagtacctatagtcaattgctattctatttttcacaggccattgttaagggaattatcggaagcttcataaaacaatgtatatcacttccatggtttttggtcatttgtttttattcagtttatgaataaatgtcaataggtttattttgtttgttaaggagagaaaagataaatttaatattttcatccaaatttagtgggactcaggtatctaataattctctggcttgacaggacaggaaagaaatagaagtagtaaatatcatttccaacgctgtttatatttttatagtgaccataatagcaaagactttatggcaatgcagaataaaactgacattggaattattataaatagtgtgtgaaataataatctttgttgtaaaaattgaattatggattttttttccagaattagttttcattaaacttgattttacacttacatgaatgattttttggaagatagggataggaaggaatcttcacatgtgttagacttaatgtagtttctaaaacgtgtggagattttaacaaaaattatgtagcctgaggattttaatagacctgataaccagtttgagcatattcttttgctgttatttacctaagatcagccagttgttatggaatcctgttcattaaataggaagcttatatttttattactgtttttggggaaggttttagagtgaaattcattaacctctaaggtgtttttacatagtactacttggaaaataaagtcattgggcaaatataaacaactgatttattttatttcattttttgaagtgtggcagaccttaagcatgccattcaaagcaaatacaagattgctattcaacaccaggtgctggtggtcaatggaggagaatgcatggctgcagatcgaagagtgtgtacctacagtgctgggacggtaggtattcaagagtcctttcacacaagaatgcaacatctttacatcacattgggaactcagtgttcaaaagtaaggtttatttgcttcatatattgaaggcaatgtgcacgctcagtatttataacattttattttggttatttctccttttttaaaaaatataggatacaaatccaatttttctttttaacaaagaaatgatcttatgtgatcgtccacctgctattcctaaaactaccttttcgacagaaaatgacatggaaataaaagttgaagaatctcttatgatgcctgcagtttttcatactgttgcttcaaggacacagcttgcattggtaagatataaacaattattttaactgattgtttctaaaagcattcggatctaaatagcacagtagtctaaattgcataggaagtatgcaaaaagtttgacaaataaactttaaaaatattttccggttaatatatctagttttaaaatttctgggtcatacattgctagttattctactttgataatgacagtattttagagtggaaaagctgtcttaacatgatttggtcaccttctttcctagataatatgtaaatagttacttttgaacattaattaataaaggacctacgttagagtagatgttaacaaagagaacttaaatagctctaaaaatgtatttattttttccatatttgtggttagttcctggaggagaattaggtttctttctaatatatagctcttaacactgctacttgcagttcccacggattgtttcccagtcatttgattttcctttttaatttactatttaccttctagtaatatcattgacattaagctatcagtggtaagctacatttttcttttaatcatggaaaaaatgtgttagaaccaatgaaataatagtatttaccttttgtaaaaataaataacattactgaacattagctttaattagtaaaactcttagagatttgttttaagcagtatatacatttgtacaaatacccccatttttcacataggatacaacattaaaaggaaataacctcatctgtgtgaaacatatttaataaacatttgagagtccaaaagtgctttgagaggacaaaattttaaaaaatatacttatgtaaactgaatttacataatagaatcttttagagttgtttttttgatttggttgaaaagtgtacatactacattttgatagagaaagtattaaatatgaaaataagttaaaggataactaggatatataattttcaagtgattagaaagagcaattaaaactatattcaacgaaagaatgaaaacaagaaagtaaaaagaaaagataaaatgtcagaaatacgtatatcaggaattaaatataagctggcattatttctcttttaatgcatcatatttaaaatattttttaaatacataaatagtaatctgagattcagataccaagcattctatatcagtctgttttcatgctgctgataaagacataacccgagcctgggacgaaaaagaggtttaattggacttaacagttcctcgtggctgaagaggcctcggaattatggcaggaggcgaaaggtgaaattggccaaaacaaaggggttacaggtcccatgcaagttcgaaatccagcatggcagccaagttttgaagctccaaaatcatctcctttgactccaggtctcacatctaggtctcactaatacaagaggtgggttcccatggacttggggcagctctgtccctgtggctttgcagggtgcagcctccctcccggctgctttcacaggctggcgttgtgtctgtggcttttccaggtgcacagtgcaagctgttggtgggtcttccattctggggtctggaggaccgtgaccctcttctcagagctccactagggagtgcccccagtagggactctgtgtgggggctccgaccccacattttccttctacactgccctaacaggttctccatgagggccctgcccctgcagcaaacttacctgggcatctaggcgtttccgtacatcttctgaaatctaggcggtggttccccaacgtcaacttgtcatttacattaggtattttattgacttattatatagagacaggatctcactgcgttgccgaggttggtctcaaactcctgggctcaaatgatcctcctgtcttggcctccctaagtgctgggattataggcgtgaatcagcatacctggcctcttttcagatttataaaatacattgagccaccatgcctgccctcttttcagatttataaaacaaattctaaagctgctataaccaaaacagtaacataggtctaatcaattcattatgtgaggaattagaggtgatatttcttcatggtccgggatgtggtctctaaagtcagattttctgagtttatgtctgattacttcatttacctgtggtgtgaccttttactcatttaccttattggtaaatttaagaatagatttttgtttattgagatatactgaaaaatgcacaaaccataattgtacatccattcttagtgaattttcacaatgaacacattgataaaaatagtttcaggtgtggtagcttatgcctgtaatcccagcactttgggaggccaaggcaggcggatcatgaggtcaggagtttgagaccagcctgactaatatggtgaaaccctgtctctacttaaaaaaaaaatgcaaaaattagccgggcatgctggcatgtgcctgtagtcccagctgctcgggaggctgaggtaggagaattgcttgaacccaggaggcagaggttgcagtgagccaagatcgcaccactgcactccagcctgtgtgacagagtgagacttcatctcaaaaaaaaaaaaaaacacacacacacacacacacacacacaaaaccccacaaaaacaaatagtttcaacccagaagccctacttaggactttctccccatcagttttcatcctccctgttcattaatgctaaccagtatcttgacttctaaaccaacattacttggtctctttttgaatgtaaataaatggaataatgtagtttctgttcttttgtgtctagcctcttttacgcaacatgatgttcctgagaatgattcatgctgttgcatgtatccgtggtttgtattctttgctgcattgtattctattgtacgaatatgccacagtttttttttaaatctgttctgctactagtaggtattgagattgtttccagtgttcaactattgtgactaaacctgctgtgaatgtcctagtacattcttttgactactgtagtgtagagaaatcagaatactttgacagtgttaaatctgtcttctcttctacttctgcaatctcttctgtaccgaaatgtccatagttctttcagatttcatcttgaaatggactcagtgcacactgtcttttttcttccatatattagctatcatttacatggtaattctattaatttcacttgtcttggttgttaaagcttgatttagacttctctgttttcagccgtgtcttcttccagtgtagccttcaatcagtgatacgtttctttttaacttatttcctgagctaaatctctgtgttgagttcttttttataaagcaaaattagaccaagttcttaggaaaagaatttggttataatttccatttgctcttggagaaagtttttctgcatttagttttttttacttttaattctttttttctacactaaattagggtaatttagtgtagatttcatgttaaaatttttgcttgtgtttgaatgaatagaattattcctctataagacagtgtaggaaagggtataagatagcacttcctggtgagcaggaagctgccctggatcacaggggatccccagttagaagcagaatctatatgtgtaagagagggatggagatgacttattttgtccttctccttggacaaaatttagcagttctagggctttttctcacgttttttcctttggctttttgcaagtatgtaacataaataggtattatttcttctgctactctctaatttcagatagggccccaagaggttcttgctgtccacccaagcttcccacaaggaggattcagttctcccaagttttgctaaatgtctgcaatagtggtgtgaacctaaattatcgtgagacaggtcacaatcaatttagaaaatttattttgtcaaggttaaggacacacccaagacatagcttcagggggtcctggtaacatgtgcccaaggtggtcagggtacagtttgcttttatacattttagggagacatgagacatgattatgtgtaagaagtacattggtttggttcagaagggcaggataactcgaagcggggtctttcgagtccttgatcagcctttcactcaatacataatttagtctggctcagtgcatctgcatttttacataaacaataaacagtaagacagaggaagcagataggcctttgtttcaggtcagcagaggtgtgactttctgtcccgcacatatgaagatatgctgtcagtttacattgtgaaggtgaaattcagcagaactgttttaggttaaagatcttgaagcctacaagaaatttccctgtggcaaaattgtgagggaggtatgtagctttcttatctttgtagctatcttatttaggaataaaattggaggcaggtttgcctgacatagttcccagcttgatttttccattggcttagtgattttgggttcccgagatttgttattctttcacagtgggtgccctttttatttgacccaagttgatcagttttcatattcttcacaggtttgctgcatatattgtatgtgtggggtggtagttgtttcctggtttcatcaagatggtgtcttgtttcccttttgtatttggttggtttcatgaaggagaagcaggcaaagacatctttacttcattatcttacaactgaaactattagccctttcttatacacatatggaattaagaacaggtgaagataagtcatttcttcaaggccagatagccagggagaagaagaacctcttttctccaaggccagtgctctttacattcatatcattgtacttccagaaagtaaactttcatattttacctgtaaagatatttcttttcacctaaatttgttcattaacaatattatcttctaaaacagaatggtcataattttggcatccttatcagccaaatatatcagcaaatatatcagcaaaatatatagagaaaatacttcctctttcgttgcatcccccactccatcttaatgatggtggaacatttgttttgcattttcctagactttgatatacacacaaaatgcatttctctctcatacctctgttgtttcacatttctaactgaccttagcctccccaaataaatgaaagcatactatactttttttgttgttgtttttgagttggagttttcgctcttgccacccaggctggaattcagttgtgcgatcttggctcactgtaacctctgcctcctgggttcaagtgattctcctccctcagcttccccagtagctgggattacaggtacgtgccaccacacccagctaatttttgtatttttggtagagatgggtttcaccatcttggccaggctgacctcaaactcctgaccgcaggtgatcgcccgcctcagcctcccaaagtactaggattacaggtgagagccactgcacccggcccatactatatatattattcctttttcattgttgcaaggtactcccatttttgaaatatcataaatttattttcctagtaaacagtgctacagttgaacttattttacatttataaattttaacaattcataaaattgaaacatttgaatattttatatcctccatcttttgtgtgtttaagaatcttcctcctacattttctacatgcgctgcaagaacttcttgctttaatgtacccaacagctcttttactcgtcatttcaagggtgtcacctctctttcatctgtttaaaaaaatctgaaaacattttatttgttatttcataattccagagtttttttccccactctattttagtggcagcatgattgattaattgacattaattgacatggcacataatcattttactgacattcctaccccataaagaccatcttacttatttactttggtaactagataacttacagtagcttttttacagtttgcctggacaacttaaaaataatcattttacatccattctttcaggtgccttcccgttccaccttatgacattttctgtattatcagtcttcttaaagcttctatttgattacattcttcatgttaaagagttcttgaggaggtcttttctgagtaaagtccaattcctccttccttaccttagtatcttctgggtctttgatcatctggccctctttctctcgctttttcctccagaccatttgttcttaaccaatagtgcttcatatttcctgtctttgccacttcgtgttccactcttttgaagtacttctttccctatctttgttcatattcttttcatacctttatacttaagttaaatgccttcagttaactttgacttctcatttcctgtctcttttgtgacttttatatctgtctcataccagttaacaaaggtagaatgatctgtcttatttccttgccaactaaaatattttataacagtggacttctttatatatctccatatataaagcaatctacattgctgccttgtaatttatccttaataaatatttttgtacttcattgtttggaactgaatgtcagatgtctattttctgaaggtattttcactgcttaaatattttctagaatctcatttagggttactaaatctgttatagttatactaatctagaagtagatgacgtattttagctttgccttcaccaaaaattgtttttaacatgaatgtcttgagataataccagaggacagtgacatttgagatggcagatgttgatcattagtcaaataattgaaagccctactcctttgtttttcagaggtagcgtcttatggattcttcttttgactgtctcttaggatacctgctcggaaacacctcacttatgtttaggctgtcttttacagaagagaacagagataaactaaaacagtcctttgaaatactttgttaaatgtgctctacaacgcatccacacgtacatgtttgtaaacatatgcaaacttctgcaaacaaaccattgccttcttttactttcttttgtattctttttcaatataggaaatgtatgaagttgccaagaaactttgttctttttgtgaaggtcttgtacatgatgaacatcttcaacaccaaggctgggctgcaatcatggccaacctggaggactgttcaaattcataccaaaagctacttttcaagtttgaaagtatttattcaaattatctgcagtccatagaagacatcaagttaaaacttactcagtatgtttgccattaaaatggataatatttgtttaaaatgcctttgaaatttatcagtaggtttaattactgcaaatttgtttcttataggccatatatttattgtaactgaggtttattctttgagccttttctgatagtgatttgtaggttatagtttttaatttaaaaaatgataattctctttacatgtatgagtctcccacatttctttctcttgggttgttttacttgtaataaggctccaaatacttttgaaatcatttataaaaccaagaatctttgcttatgtcttgtccactaaacaaaaggcaagatgtgaaaaataacatctaacagaaataggcacattatcaaacactggacaatggaggaaaattttaagttctttgtaaatattgccatcctgatacaggtttgatgaggtggaaaaaaagcccttgtgttgtctgttttcttttcttttttttttttttttttctgagacggaggctcgctctgttgcccaggctggagtacagtggtgtgatctcagctcactgcaacctccacctcccaggtttactccattcttctgcctcagcctcccaagtagctgggactacaggtgcccgccaccacgctcagctaatttttttttttttttttttttttggatttttagtagagatggggtttcaccgtgttaggcaggatggtctcgatctcctgacctcatgatccacccgccttggccttccaaagtgccgggattacaggcgtgagccactgcacctggccagcccatgtgttttcaatgctcattaagatggcaagttttagttctttgcatagaagtacatttaatacacattgattctttttttaaaagggctgtagttaacctgaaaatataatggtaattcagcatgaaactaacccttattgtgtgtgtgtgcatatgcttttagcattttgtgttcaggttttttgttttggatctgtagatttttttttcttttaattatcttgaccttgcattagaggcaagtagcatgttctggttttaatctttgtctttttgtttctttgatttttttaaatttgggtaaccttttaaattcagatttctctgttttgtggtatgaatattagtttttggccttaaaaatggaaaaatcttgagaggaaagagaaggattcattaagactagaaagatctttttaaagtttaaatgagaaccttttccagtaactttctaagggttctgttagctgtatcagtttaaagtagggacctacatcatattgcacatcattttatagaagttaaatcaccaggatttcatattttccctatgcttttgacaacataaacctgttttcagggtgttatgagtacaagagttatcttcattaggattgttatttttatcctgtgctttgaatatatcaaatatatttgatttacttaaggccataaggttatattctaagatactgctatggtgaatctttttgtaaatttaaatttgttacatctttaaggttttgtaaaagcctagtgttcacttgtttagtatagctactttactttagagtgttttttttttttttttgagacggagccttgctctgtcacccaggcggctggagtgcagtggcaccatctcagctcactgcagcctccgcctcccaggttcaagcgattcttgtgcctcagcctcctgagtagctgggattgcaggtgcctgccaccacactcggctaatttttgtacgtttagtagaactgggttttgccgtgttggccaggctggtctcaaactcctgacctcaaatgatttgctgccctccacctcccaaagtgctggcattacaggtgtgagccaccacgcccagccctagagtgatattttaatgggttaacgatagcctctctctgctaaactagacttttagtgaatagctagctcatagactagtttatgtatgtataatgttatggagaggtggtgagatttgtgtgtataataatatgtgatttcattcaaacatgctaattcctaattagagcagtctcatagttttactgacagatcttttgtattttaagtttaggaactgcagtttcagtaatggccaagattccactgttggagtgcctaaccagacatagttacagagaatgtttgggaagactggattctttacctgaacatgaagactcagaaaaagctgagatgaaaagatccactgaactggtgctctctcctgatatgcctagaacaactaacgaatctttgttaacctcatttcccaagtcagtggaacatgtgtccccagataccgcagatgctgaaagtggcaaagaaattagggaatcttgtcaaagtactgttcatcagcaagatgaaactacgattgacactaaagatggtgatctgcccttttttaatgtctctttgttagactggataaatgttcaagatagacctaatgatgtggaatctttggtcaggaagtgctttgattctatgagcagggtaagtaacgttaatttgatgtttgttttgtcattttactactttatattatttatcctaaatattttatgtactgagagaatgggtattaccttttagtagaaataaatgagtcttggagtgaaacttttaacataggttgaatcaatttaattcataattttatttatgaattgattgtaaatttgtgaaggttatttgagattaataggaaaaatttctagtgcaataagaataatttacatacagtaaactacacaggtcttgagtggttcatgcaagaaataaccaccacacaaattaagataatgaaaatttcattgcccagaaagtccccgtgtgactctttccagtcactatttccttagaggcaacttctttctgacttttaaagaagaaatttcaaatgctgcatctgcctgagatctttaaaattcgaaacgtagacactgtattttttagttgtgttttatagttatagtgagcttgtattttggattaaggtcttctccagcacacctgaaaaccttttaaacctgaggaataaataccccttcatatctgatctgcaaactgatgcaaaatctttgacaccaggaaggaagttgatggggcatgtttcattgttacagaaatgttaaataatgttgtgaaggctgctgttgtcaaaagataattgttaaaaatagttaaacttttcaaggagcatgcagtcgaagaagagttcatctttgattcactatttagagcaggatagcacattgtctgggatgcctaaaaaagaatacatacattgctgttaaagttataggatatagtaaaatggaaaacaaaaaatctaggttttgtcattctttcagtatccagtttcttgatgacattctgtaggctaagcatttctttcaattaagacatgtttgccagagaagtatgtaatatgttatgcaaagattgatcactgccaaaaatgaattaaagttagtgttcctgcttaaactattgacaatgtagtggcctgaaaattccatttatgattgtagataattgcttacatgattgtgatcagaataattaaggtattttgttttgttttgttttttgagttagggtctcactctgtcgcccaggccggagtacagtggcatgattatagctcattggttctccacctcccaggctcaggcaaccctcccacctcagcctcccgagtagctgggaccacaggcacacaccaccatgtccagctaattttttcattgtttgcacagacggggtcttcttcctatgttgcttagcctggtctcagattgccgggctcaagcagttctcttgcctcagccttccaaagtgctgggattacaggcatgagccactgcacctggcctatcattttttctgtaattaagctgtgccagatgtcaaaacagagttatatatatgaagcgaacctgaaatcggctttgtatttccatgttcagtgtgaaatttcacggagtcttaggtttttttcaatttgtatgactaaaggaaaggaacagtgattctctgacttaatgaaaagtcagccaggcacagtggcctgtaccagtaatcccagtgacttgggaggctgaggcgagaggatcacgtgaggtaagaagttcaagaccagcttgggcaacatagtgagactctatctctaaataatgaatgaaagaataaatgtcagaaatatttataccttcagaaaatagaaggtagctatagtgttattactaagcaatcacttttttctttttagaagttaatgctacattgaatagggcatagcaacaacatctgaagtctttgtgatattttacatgaccattatattaaaaaagcaaaatacaaacttgtatttggttattgttgtttgttatcaaattagctaacgtgactttaaaatagtgcctataatatagtactcattagttgtatatgcccattagattttttttttacttactctatcaggaggagtagctattggatgctgggcttaatacctaggtgctaggatgatctgtgcagcaaaccaccatggcatacatttacacatttatgtaacaaaccagcacatgctgcacgtgtatgccagaatttaaaataaatgtttgaagttaaaaaatatatattattctgaagcagttaaaatccaatttggaaaaatttatttatattttacaaagtatatgatctgtaaatgtgatgtattgcgtaatgaatggcactttaccatgatgtttttattagatcagtatttttctcctctcaggagtactatactttttcttgataagactaggggcatttctgaagatgaaccgagtatgagatttctctctgattggagatggttttttactctcattttattaaacggttgtccaaaacaactaacctacctgatatctgatcgcagtagcactgttttccaatgaagtaataaaaatatatgccaagattttttttccgtaaaaggaaattcagtttaaatctatcgtttttgcagattaattccaattgtaacagtattttgtaacatgtactttcaactggcaaattaaaatgtctttctcaggaaattagtcgagacataggtattttggaaattaggatttggaattggaatagcaggggctcaaatctgggcattgagattcatttactatctgtcatccataagtcacttaacatgggattcctagttttatatttttgtggattaagagatgctagttttgtaaattattgtttttccccccattttatctgtcagaaaaaatttagaattgttgctgcattttcgtagactcatatttttagattttagtgtattcttgctaaaaagactctttttttttttttgagttggagtctcgctctgtcacccaggctggcgtgcactggcacgatctcggctcactgcagccttcacctcctgggtagctgggactacaggcacccgccaccacacctggcaaatttttgttttttttaatagagatagagtttggccatgttggccaggctggctttgaactcctcacctcagttgatttgcctggctctgcctcccaaagtgctgggattacaggtgtgagccactgcacccggccttaagatgactcttgagtgttctgtttcagtttatactacaaaaggcacactgatttgtgtttctaacgattaataattggaagtgtctgcatttagcacatttctaaactgtccaggttaatgcagcataatttattgagctttagcatttctttcagacaacttgtgatcttaaggaatctatgcaggggagtgggtttaaaggtcagaggcttaagaatagagagtgtaagaaactttgttttatagtttctttctcagttaaaaataatgttagcctttgtagatgccaaagagcccaagagtaaaaattaatgtatcactgactgttaattattgaaaattggctaaataattactaatgagattctgtggtttacatatatgtaatttaaaacatttcttcaaagatttttattatctgtgaacaaatggaataaaatattaagtgcagtgagattttaggaagtcagctgggcacggtgactcatgcctataatcctagcactttgggaggccaaggtgggagtccattgcctgagcccaggagtttgagaccagcctaggtaacagggcgatacccatgtctacaaaaaattagctgattgtggtggcacatgcctgtagtcccagcagttagggatgctgaggccgtaggatcgcttgagcctgtagtgagctgtgatcatgccagtgcactctatcttgggcaacagagccagaacgtggctcaaaaataaattttttttaggaagttgtgtaaactatttagtgaatcataaaatactctcctatgtttgtcagtgcagttatactttcaatgaattattaaattcagatacgttgtattcaggagcccttgtttttggtttaagttgttggtatgttaagtttatgtataattgtaattttgcctaggttgaacatttgaaccagttttcatctttatcatgctaataatttgtaaccaggctttgatgtgcatagtactctggtgtagaaatacatacacattggactcagttaccacaaaattgagaaaagtgagaaaatgttcacaaatgtacaagtagcagttgagacaacattgtgcaaaactagtggtgcctattctgaaataatggttttgttaattttttcggtagtgttagacatgcaccctcttaactacactggattcttgataataacaataaaatagtttagtttaattgcttttgtatgttctctgtgtacaagactctatgcattaatcagttttttccgactgtagagaaggcagcatgaaggaggtatttgactaggatctttagagggagaggttgacttaaacagttaatagaagaattgtgaacaaagatcagagatagaaaaacacggtttatcttgaaatgatagacatttcagcttgaaaagatgagaggcttgaaagatttttttctgatatcagatcaatgaagcctttattatattctacatttattggatattatctgtgggcagaagaaagtcatttgagggttttattaggaacaagcatagtaagtcaataatagaaggtttcatggttgctacccaaatgtagatgttagtaaacagtcagaaacacagtttgagaatctgtgagactgatcagtgattgaagtagacttgggagttatctccagaaaagttatacttaatgctgaaatcatattgagaacagtaaaaatgggctaaggacaaaatccttctgaatcatctgttttagaaagattagacagagcaaaaggagccagaaaaggatgctgtgaaggaaccagtagaagtaacttcccagagcctttaaggttgggaaaaggttatctgccatgctaactgttgaagagcctcaagtgaagcaaggtcattggtagtttttggctgagcagtttttatggaaaacgcagacagaaaggctgccaggtaaaatatttttttaagggaccaggagcattatcggaaaattttataggttccatgaatttcagcaaatgtgtagaattagagccagggaacaggagagatggaagagtgagaaagcataattgagcaagccgattcgttgaggagatagaaatagagaataaggaagttttatcttctatctccaggagggagataaatttgccgaagaagtttatatgttttaaagagaagttgcattgataagagaattaatgatattatgctgacattgaaaatacctttttctgagagatattgacaagtgattaaattgtagcactgattgaaatgtgtgattcttgaaaatgtattcctaattatgtattctagtgtaaattcagtgttgtcaaggatatttatcctgtacttgaccttcttatgaaagtataacttagagttcccatgcttttgcttttttatatcttttatcacttattttctctcaagctgtatgtcagaatagcatgaaatacattttaaactatttgctgaaaattgtattcgtatgtgtttccttgtcatttagcttgatccaaggattattcgaccatttatagcagaatgccgtcaaactattgccaaacttgataatcagaatatgaaagccattaaaggacttgaagatcggctctacgccctggaccagatgattgctagctgtggccgactggtgaatgaacagaaagagcttgctcaggtacctattttagacttttctcaggaaaaggaatttatagtttttttgtacacatgttttatatgcagtatcaatttggtgtgcaccatattttcactttttccttgaatatttttaatgtattaatattgtttccttgtaataaaacagaaaagcatgttcttaatgattgagtttcctatagataaaccatgaaactgctacttgaagctgggggcagaaatcaacattttttgatatgttgaaagcagatgatcatatcagttattgtgctaaaaatttcaatgcttcctactaccccccataggacgaaggataagaaatgaaattttttaagaataggagaatacgaaatcttgaagaatttactaatacttctaatagtaatacctaggttttagatctgatttaatgccataatcctagaactgttgaaaaagtggattttaaaccaaaggaaggtgatggaatggctaagtaagagtggtaaatgtggacgtttctaagcaaaccagtgcctctcttaaattcatgattgcctgtccttcacccaccatggctagcattgtggcaaaaccacccatagctgaatagttgaatgtcataatcagattctgacagctgtttttctagtttaattttccttgccaatatttttcctctaattgtgttctttcattggaacatataaataattacatcccccatattaacttttccttttttttaaggcaaaacatacttcgttgatgttatagttaaggctgatataaattagaatgtcctttagtatttccttttagtgataagttcaattttgttgtttgaaaatctcttaaatcaccttgatttctggaggatatttttacctggttcatttaaaattctgggttgttagcattttacttttggtcttttccttctgtttggtttttagcaattttagtatgaggtgccaagatgtggcattctttgtatttatcctactttgggcttgtagtgcatcttgaatctgtggtttaatgacttataccatttgggaaaaatctgagtcagtttctcttcagatcttgcttttccctcacttgcttttccttctctttcagtgattccagttagacgtgttagacctttcaccatatccgtttcatttacacttgtttcctgtattttctttctttttatctatctgtgcttcattctagatattttcctttaacctacattttaactttttctctttttttagtggctaactgtagctaaccacttgctcatttcttaattttgttcatttttcagctctacagtttcagtttggttcttttgtatatagtttcatattctctacccaaatatttagtcttagccattcatcatctctttgatcacattaagcacatagcagtttaaaagtctgtatctgttcactgtattgcctgttcctgtgtgtctgtttatatttggtggtgtttctttctttctttcttttttttttttttgagaggaagtttcgctcttgtagcccagactagagtgcagtggcgctatctcgtctcactgcaacgtctgcctcctgggtgcaagcgattcttttgcctcagtctccgaagtagctgggattacaggcatgcaccaccacgcctggttaattttgtatttttagtggagacagggtttttccatgttggtcaggctggtctcaaattcctaacctcaggcctgcctcggcctccaaaagtgctgggattacaggtgtgagccacggcgcccagccgtatatttggtggtgtttcttataattctcactcttatggtcttgccttttcatgtacctgattttttttttaataagaaaaatcaaagaaatattgtgaggcctaggataacgttttcttctgcagataatttatgtttgcttttggtatgtgctttgggcagttaccatcgtaaattactttagttcagtttcagagatttggatgattggacattggtattctagttcttgggatggccattctacttttagtttacccctaatcataggatataggttttaaggatctgaacgcaaataaactgttccattgtaccttcccttggctggctctggtcttcaacttttaattttctagctcttcagggaagtcaggactgctgactacttatttttaattttggactcctcttcaagaatcagcagaattcttcagagcaaatggcattcctagtcacctaatttctctctcttggtttcgttcttctcctggatcctagccctttcactgtgcctggcttcttcactgctttcctagagccacagggagcttgttgacattacaggctaccattactagagacagaactctgcattcctgatcttttgcttcttctgcagtgtagttgttttttaacttccatagaatgaatcaaacaaaatttatttaaccatacttcttttggtgattatttagtttgtttcagtttttccatattagaaatattcattgagtatctcaatacacccatttttacatgcatgttctagtatttctttaggcacaatcttcaataatgggattgttggattaaggtctttgttcattttaaatattaattaataatgttctaatgcctgccatagaagcaaaatcagtcaacacttccagtaagtaagaagagtaatttctctgttttcagcgtttgccatgtatttactctgaattgtcattcgccacattttctttttcatgtttttgttagaatttatcatatattgtctagatagttctttgtctattatagatcatacagatattgtctctgttgataatttatctggatcgaaaaacagaagctttaaattttgatgcagtcaaattcattcgtatatttattcatattaaattttatgaacccatcattctataaaatcgtctagatttctttataactgtttctcagattgaataaactagggctttcaattatcttaccttagacactcgatttttctctctctactttttctttgctccccttaatctctcagtatattaaaatattgctattagtcctcctgctttatatcacttgaacagtcaatcaggcaggtacattggatggttttatatttgttagtaatataaaaagatatttattgctgatattgccctcaagtctacttggtttcagcaaatggagttttgtcatacaaggaaaagtctttttctgggagtatggttggggtgataatactgttttctcaaaatggaagaatgcattatttgcgatttgagcttttcttgtttgctttctaaatctgaccatttaacctccctgaatccaaactgaagtaaaattcagtgaaaatttcttatgctgttcctatggatattacaaaattagtttttgttgtcttgtgctcatagagcatcctctttcaacatcttcattccccctaatttatttgtagtattttgctattgttttaaactatggtagctcatggaatataccaaaagaatactataggtaatattgttacttttagaaaatatcttttctttcttcgatacctgagtggtatagtccttttaaacatttcgagacaattaaatatttaattatttaagttctccaatggatattttttggtactcctaagtattcataagaagtcatctacattgtctggttgtatattgggagcaaaatttaagacagaaatatagaccccaaggtagacagttttcaattgagtgtgcctgaatgaatctgaatgtgataggattcagtttaataagattattttcagtgagctcattagagaaaagggatacccagttgaagctaggaggtatatggtgagtgagacaacttttcacacctatattgtcatgtatttggctgccgccaagggatgtacagagtcaaggttttagcagaagcatcacttcctcagccaaactttcctgatcttgaagtcttggtcacatcatcaggttcattttctcttagtatgctgtacttatcctgtaccacagttgtaattattaattacatgctgtttaatacagtctcttcccagaatataagctccaaggaggcaggacactactctatcttccattgctttatctacaaggtcttaccacacattcctgatacatacatagtaggaacacatcaatacttaataaaatgagaatttggatttgtgtttttttgaatttgagaaatgtcacaattctatctagtctttggctggtctggaatttttatttacagatgtctcatcagttacttttgttgctgctaatggtatttccctgttgcagatagtttgtcagtacttcctttagaatgtgtttggtgaaaagtagataaattcagattatccaattttaggagttgattttacatcttgctaaacacatttacaacgtagtataatacattctagctacatagcaattaaaacttaaaaccactcccaattaaaagggaaaagaaattgtggaaagggatttattatttattgcctgaacatctcataagatagagaccatttcaaatgcctttgaaggggtaatttcttaaaaccacaacatttatgaggattgttgtagacccttctacatggttttggcaaaaaaaaaaacctcactcaccaaacctatgaagctgtgtaacactggagccctgaatattcattctctagccattcgtatccttgagggttatcagcaaaattaggaaatatcagggagttttcttcctgaaccaaatttggctgaatgcatctggaacacgattgtgagtactacagtgctatcatgtagctcatgtagctgtcaaaccttatttgcgtgtaaaaatattagtaatgaattagtactagatgatttatgaggattttttctatttagtctgaattatattgagtatggttgagcaaatatgaaattcaatttattgaaaaactttattttttccttcttttagcaatacagtacttacatgcttttggtgtcaggaaatcctttactaatggtctagaatttaaatttgggattgattctatatttttcatggattcaataaaaagtaattgaaaaccagactttgcgaaaggagttaaaagtagcaaattgttttttaatagattacattatagaaaaattatagttgcagttaaccaaagagtatattgaaagggaaaaaaaaggcatacagttactatgaaaattgtcttcctttcagatattgaaccagtttgacttcagtatatatcattcaaatctaaatgtcatgatatttaggaaaacatgaaacagagaactaccagaaaattggaagacacacttgtcctttagctggtcataaagttaagttcctagaaggataatgatttatttacagtatcaatacagcaatcagatggatgtacaaaacctacaacattattacatataagccacttggtatatagcataggaaaaaactttgtggtagcaataaagtatgtagtaataaccttattaagttatctttggaccacatataaagaaaactaaatatacttgagagataaatgaaggaagattatggcagatctatctaaattaattagtaacttaatccctacttgacagatacaccgttcacctacaaaaacaaatgagtaaggacagttaagaaaaacagaaaaaatgtttttctaccaaataataaagtataaactctgtgtaaaatcacacaattcagtcattgtcctagcaatgggagcacagatcagtggaacaaaatatagtccagagataactggagagggcagtgacttttcgataaatattttaagggcattttattatttttatatttgggaaagtgtctaatatactactgtataatgtttgctagattaaatcccttattgtttttcagtgaccttgttttgtttgtgctgtgactgaccgatagtgttataggattctagacacatagcttctaattctcattgactattaagtatgtccttggctaaggggctttatctttctagcctttattttcttctagtttgaaaaattaaagatgagcataggattcttaaaaattctaggtcttgtgccatttatgcatctactacattcgagaaaaatagaggatttaaaaatattgttagaaggcacaaaacgggttgtgtttaacataaagattttccacttaactgaatgtccatacctttcatgaaacaaaattaatgtcctttgaaacattttttttctcaaagggatttttagctaatcagaagagagctgaaaacttaaaggatgcatctgtattacctgatttatgcctgagtcacgcaaatcagttgatgattatgttgcaaaatcatagaaaactgttagatattaagcagaagtgtaccactgccaaacaagaactagcaaataacctacatgtcagactgaagtaagtgattcatttatatctgttttaagatgtttagaattggtatttatatttatttctcccaaatgatggagtgatttttccctaccattttattgaaataccttattgatgaactttggaattagactatttttgtagagcttatatatcttgatttctttggcttaactaagttagtaagcttactaagtaagtaactaagttagtaccagttttgtgaatgttgaaaataatggattttattctctaggtggtgttgctttgtaatgcttcatgctgatcaagatggagagaagttacaagctttgctccgcctcgtaatagagctgttagaaagagtcaaaattgttgaagctcttagtacagttcctcagatgtactgcttagctgttgttgaggttgtaagaagaaaaatgttcataaaacactacagggaggtatgcaagttgaattctattttaactgtatatgaactttaacgaattggtgtatgtttatgtttaacataagctttcctcttctagtgggctggtgctttagtcaaagatggaaagagattatatgaagcagaaaaatcaaaaagggaatcctttgggaaattatttagtaagtgttcattaataattttaattacttttgtaaaggataaaagtttttataagcttggattttctgtagcatacttcatgtatatgctgttaattgccattgatatctaggaaatattccttttaaagtgtatagaattgaatccattgttaaattttaaacttgaagtactgtcaacttttaatttaccttttcaaaaaccagagataattttttgccttaaataaagctctaaaataaccatattgttaatttctttatacagggaagtcttttttaagaaatcgtctgtttaggggactggactcctggcccccttccttttgtgtatgtatttattttctaaccatgactaaattccatatgttttaaaaccttttttccagaagtagagacatttatcagaaacttgcagaaattgttgtggtttaaaaaaaatcaaaataaaagtgtttttggctttaaaagccagttagcataaagctttaagattgatatttgtattcataattttttataaagaatcttcataacacaggaagataccttatactgcactgttaggattggtagaaatagattcacttggtttttttgttttcttgtctcttcctttttttctttccacccttccctcttctttcttccactgctcttctccctccctcctcctttacagcctccacctcctccttgcccttctatatttcttttcacaccacccccctttatattttgacaaatcagaataataaagcctcagttagacttcactggaattaaacgttagtttgatatgtttctaaaccattgttatctggggtcattaattgtattacatgagaagaaacgtttccatgatcaaagcaatatgggggcttttacccccatcagatgtactcttagctgtgttgttgttgttttggagacaagagtcttgctctgttgtccaggctggagtgcagtggcacaatctcatctcactgcaacctcagcctcctgggttcaagctgttcttgtgcctcagcctcccaagtagctgggactagaggtgcctgccaccacatccagctaagtttttgtattttagtagagacggggtttcaccgtgttggccagctggtctcaaactcctggccccaagtgttctgcccaccttggcctcccaaattgttcaggttacaggcgtgagccaccacgcccagccatgcttagctgttaaacatgtttctatactgcaggatttcttagtgttttaaatgtatgcattttttaatacataagaattggtgacatagcgattcccatacttatttatccgagaactcttttttatgaagagcatcttggcatctccacatctgcttgagtaatgatttaaaacaagtttttttttaatgcaactcttggcaattttataaataatgaattggtaggatttaccggttaatattttttacagaagctaaaattgaccaaaactttgtcttctggaaaatgtaatggaaaagtcaaggcaaaagcaaataagaaagtgttacatggaaaaaaaatacatgtgtgcattttaatatattagtggctttaacatggtaaatattatatatatacgcacacacttacatatgtgtttaccatatgtatatgtacatacatacacatacaggataggaatattgtaattcccataaaatgatgctaaaagcagagaaggggaggaacaagtgcatcccttgatcactagcagttcacgtcgcagtttactaaaggctcaggaaaataatatgccatatctttattgaatggtctttttttgtttcttaatcatttaaagcactttagtttgtttggtatagtaaatttaattatagtaagaaatcatataaatttgctgtctgtttttggttgaaaaaaaattttttaagtaattaatttttttggtacagactcaaaagcctcgaaagtttgactgtgaacttccagatatttcattaaaagatttacagtttctgcaatcattttgtccttcggaagttcagccattcctcaggtaaatgtcattagtattttcagttaatttataaccttaaaatgtttttattattctggagttgtgccaggtagtaccagtaatcttaagaggtgccaagataagaaatatttaagatagactggctttcctgtgactgctttgagaaatttctggggaataaaaaatgatggaaaatgaaggtaaagtaaaggaagacatgccttacatgtaattatttttttctagtagttactttttgcatgctgttgggaaatttctgactttttacttttttttttttttttttttttcttgagacggagtctcgctctctcgcccaggctggagtgcaatggtgtgatactggctcactgcaaccttcacctcccatgttccagcagttctcctgcctcagcctcctgagtagctgggattacaggagcccaccaccatgccctgctaatttttgtatttttagtagagatgtggtttcaccatgttggccaggctggtctcgaactcctgacctcatgatccacccgcctaggcctcccaaagtgcttggattataggtgtgatccaccacgcccggccctaagtttttactttactgattatgacacaaacttcttaaaatacttctggtattaatttaaaaaggaatgataaggctcttgttatttttgacatattaaaatatttggtagcagttatctactagttcattaaaaaatcagaagtctaattgcattgcattctgtttcagggttcccttactttgtgactttgaacctctacaccagcatgtacttgctctacataatttggtaaaagcagcacaaagtttggatgaaatgtcacagaccattacagatctactgagtgaacaaaaggcaaattcaattctttcaaacagttttcttcattagtgtaaaatgtttgtgttcctgcagctctttaagcctttctttctgttttccaggcatctgtgagtcagacatccccacagtctgcttcttcaccaaggatggaaagtacagcaggaattacaactactacctcaccgagaactcctccaccactgactgttcaggatcccttatgtcctgcagtttgtcccttagaagaattatctccagatagtattgatgcacatacgtttgattttgaaactattccccatccaaacatagaacagactattcaccaagtttctttagacttggattcattagcagaaagtcctgaatcagattttatgtctgctgtgaatgagtttgtaatagaagaaaatttgtcgtctcctaatcctataagtgatccacaaagcccagaaatgatggtggaatcactttattcatcagttatcaatgcgatagacagtagacgaatgcaggatacaaatgtatgtggtaaggaggattttggagatcatacttctctgaatgtccagttggaaagatgtagagttgttgcccaagactctcacttcagtatacaaaccattaaggaagacctttgccactttagaacatttgtacaaaaagaacagtgtgacttctcaaattcattaaaatgtacagcagtagaaataagaaacattattgaaaaagtaaaatgttctctggaaataacactaaaagaaaaacatcaaaaagaactactgtctttaaaaaatgaatatgaaggtaaacttgacggactaataaaggaaactgaagagaatgaaaacaaaattaaaaaattgaagggagagttagtatgccttgaggaggttttacaaaataaagataatgaatttgctttggttaaacatgaaaaagaagctgtaatctgcctgcagaatgaaaaggatcagaagttgttagagatggaaaatataatgcactctcaaaattgtgaaattaaagaactgaagcagtcacgagaaatagtgttagaagacttaaaaaagctccatgttgaaaatgatgagaagttacagttattgagggcagaacttcagtccttggagcaaagtcatctaaaggaattagaggacacacttcaggttaggcacatacaagagtttgagaaggttatgacagaccacagagtttctttggaggaattaaaaaaggaaaaccaacaaataattaatcaaatacaagaatctcatgctgaaattatccaggaaaaagaaaaacagttacaggaattaaaactcaaggtttctgatttgtcagacacgagatgcaagttagaggttgaacttgcgttgaaggaagcagaaactgatgaaataaaaattttgctggaagaaagcagagcccagcagaaggagaccttgaaatctcttcttgaacaagagacagaaaatttgagaacagaaattagtaaactcaaccaaaagattcaggataataatgaaaattatcaggtgggcttagcagagctaagaactttaatgacaattgaaaaagatcagtgtatttccgagttaattagtagacatgaagaagaatctaatatacttaaagctgaattaaacaaagtaacatctttgcataaccaagcatttgaaatagaaaaaaacctaaaagaacaaataattgaactgcagagtaaattggattcagaattgagtgctcttgaaagacaaaaagatgaaaaaattacccaacaagaagagaaatacgaagctattatccagaaccttgagaaagacagacaaaaattggtcagcagccaggagcaagacagagaacagttaattcagaagcttaattgtgaaaaagatgaagctattcagactgccctaaaagaatttaaattggagagagaagttgttgagaaagagttattagaaaaagttaaacatcttgagaatcaaatagcaaaaaggtaagaattaagtttagtctgtaattataaaattaaaatatcagtgattcgtttgtgtgtaatcacagtaccttgatttactcatttaccttgggtaaatgtagcaactagggtattttcatgaagtaaaaacttactttttagtcagtgtttcttgcaaacagaaataacagactaaatccatgagtggcttttaaaaaatgaatcagcagtttggattttcagagaaattttttgttgaaagtttgaaatgttttaaagagctctttgtttttccttttttaattcataagtttctctcttgatttatttgcagtttttttctaaactcttcagtagaaaattacttatattctctttaaatttagtatatatattaaagactaaaattttgttctgtatattgtttactaaaggctaaatcataggaacatgtagagtgttccttattgttattttcttaatacagtttcagttatttcctctgtgcttgaaagatgtttttaaactgcaggtttgatactgtggttgtggattggtggtgtttttacatgtttattattgttactgtctgatattactgccttttgatcacagaagatggtttttagtatttttacttttaatctgggtttaattgggttttgtgacccagtaaacagtttctagaacataattaatatgtcaattcattctatcatgaattctccaagtgaatacatatatatttcaacctgtgtcttgccttatcacttttatacaatctcttgtgattgtctgctctataatactcatatattggtatactgttaaacctccccttttggcttaaaattagaagctctttaggtattctgggggagggggcattagagggtttaatagaggaattagggtttgcatgtatgtttaaaagctggatgagagaaggttaacagagtcactgccaaaggccaggagagcttgtcaaagagaatcagagatggacagtacttaaagcagtaaaaacagattatattcagacactattgcagtatggaaaatagacctcattatagaactaggctcaattctgaatacagcatgaacaagttgagatttatagctaaggagggtaggggtcagtggatggaaaattactaagaggaaacataaggaataaggggaattttggttaaattgatctaacaggattcttactgaaggcaggccagggcaataaaataccaagggtggggacgataatttgttcagatattgatggagagatactctctaaactgagttagcaagacacttgatacagctggacagcgtaggcccaagaagaatagatgtcaaggtcgaagcctagaggagcctgattgaagtttagttcaggagagcccttttcaagctcaaagtgaaataagaggaaaacagaggctgaagcacccatgaaagctgatggttctccatggctcactgggaagctgctacaagattcaagaatctcttcaaagcaccagtctgactcaatctgcatcacaaaatggatagttctcagttacttgccaggaagctactgtgaaactgcatctggctacagccacctctggagaataaacctgtgctgcttttctgccttccaaaaccttgcacatcttcttttcattggcaaactggaagctatacctgggattttgggaaatgttggatctctgtattgcagtgcatatgagatcataaaagaggagcaagggcaaagcctatggaatactagacagtccagcactttataccccatttaaaactcagcatttagacagataaatgtcttttgcccatattgagcttccaaataaagacacttaaaataggaaacagcagttggattgaagacaccacctgaagaaattcattaaaatccattgtttgtctccatgtcttctggacaggacacatacttaatgttgaataagaacatggtagaaacctccctcccctgtgtcgtgaagtgagagttttaggaattttacttggccttttctaccataatagtccttgttccacgagtaagggaagcaacatggggattctgccagtttcttctgtctattccagtaacgattctggatctaaggatgtacagggtgagtatcccaaatccaacatgctccaaaatctgaaactttttgagccctgacatgatgcttaaaggaaatgctcattggatcattttggattttggattttcaacatttgagatattcagctggtaaatataatgcaaatattccagaatctgaaattcagaccacttctgctgccaagcatttccgataagggttattcagcttgttcctgtctcgatgagggaaccattgggcccaaactctgtcacatgagcaccaataatctcctcctcctagtggaccacagtcacttcagtcttctcagagccagtgtttgggtgaaggttagaattctcttggtaagttaagcctctgaatactagatctagagttttctgttttgtcttgtttttacagttttagtaggtctttaaccggttcattctattagggcctacggacaaccactaccacagatttctctgggtcaaaaccttctaattactgctctggctctgttgcttgtgatagagccacattatgtttctggtggtgaagtgccactaactggcctctgccactccagaagcctgtcattcccattaaattcattgaatttcagtgttctcatctccctctgctgtttcctgcagagcaccactacagagccttttaagcttgtattttgcaaagccttggtaaagaacatattctctgggacctctgagggtataagtaataaatcccttccagtatacctgcctttggatgccttccttgtagatcaggagtcagcaaacttgtttttttgtttgttttttgtttgtttgttttttgagacggagtttcgctcttgttacccaggctggagtgcagtggcgtgatctctcttcaccacaacctctgcctcccaagttcaagcaattctcctgcctcagcctcctgagtagctgggattataggcatgtaccaccacgcctggctaattttgtatttttagtagagacagggtttctccatgttggtcaggctggtctcgaactcctgacctcaggtgatccacctgcctcagcctcccaaagtgctgggattataggcatgagccaccacgcctggccagcaaacttgttttttaaaggtccagatagtaaaaattttagcatttgcaggtcacacacagacttattcatataatctgtgtgcatgcgtgtttaccatcttttaaatatgtgaaaaacaagaacagtggctctctaacttctgtcacagtatctttttacgctctcaaaaattgactccagactgattttgttcatagggattatgtcagtatttctgtactaaaaattaatggcaagaaattgaaacttagtcattttcaaataacctcattacatggtggcatacataagacttttttttttttttttttttgagacagagtcttgctctgatgcccaggctggagtgcagtggcgcaatcacggctcactgcaacctcccaggttcaagtgattctcctgcctcagcctcccgagtagctgggactacaggtgcgtgacaccacgcccagctaatttgttgtatttttagtagagatggggtttcgccatgttagccaggatggtctcgatctcctgaccttgtgatctgcccacctcggcctcccaagtgctgggattacaggcgtgagccaccatgcttggccatacataagattttttaataaaaactttctgaaatgaaaactagttagaagagtaccattgttctatgtatttgcaaatctcttaagttttgattgaagtatatgaaaaaaaatccaaccttatgctgaatgcaattggaaaaggaaaggaatatttaaatagccttttcagatgattgtcgatattcttctttgttactaaatcaaaattcagcaagtggtaatttctgaacattagtttttattgtggaatatgcagctattagtggattttctcataatcttaacatttattggtacatctcttgtacttttttactcatgcatgattttatagcgtcatgagttggtcatttggaaaacacgagttcacggagttaatgcagatcttccaaatgttaacatattttagagctagtagtttttactattcaagaatattctaagaaaaaacagcctcccctttttcctccccactttgagtgctttgtggtgaaagaaaacgactacaagtaaagtttggtgctaatggctttaatttgtgttcaggtaccagcagtttgatcatcagtgccaatatcaacaaggtgaaaaaggcaacacttggtgttactatgaaaacagttctgacttcacttgtcctctgaatgtgtttggttaaccccaggcttctatggatcactctttgggaatcattagcctaaagaaatggttggcaaacttttataaaggcccagatagtaagtagtttaagttttgtgggccaaatggcctctatccaaactacccaactgtaccattttagcaagacagcagtgagactatataaatgaatggttttggctgtgttccgcaaaaaagcggattggatttacctgcccttgatctagagtatctcattttgccatctcattttcatttaatgttggttattcttaagtctactttccaggcagtcaacttggcaaagtgttaatagccaatattgagtctggaatctgtggtcattacacccgtataaataattttggcctcactccacattgtccctcctcttgtaactgtcattagatctattcccatatgtattttccatttttctgccaaaatatattaaagattctgtaattgctttgcaattccttctttgttagatttttaactctccagagatctggcaatttgagtgtagcaatgagtatggaagtaattaaatggtgggagaggttgttgaaaaggatcatctcattgagaagaggactacctcaagtgaagggacactgatttcctcagacaaggtagaaaaaggctgttgcttccaggaaggaaggtttggtgggatttggaggcacagctgggacatgtctcaaagccttgccttccttagtcactggtttttcaaacaactacaagtgtgtactgttatgccatgtactgccctggttttccagtgttctattttgcactggcactgaggtcattactgctttcagatcagagaattaatctcagtttccatgttttatgggatttttttttcctctagagtcatttttttgtatcaggattgagcttataaaacaaccacttttaaatatttcaggattaaagggattaatactagatttagagtttacatgaatgttgggagaactaggagactggatagtaatctggtgaaggccaagagatctataatagaaagtcatctgaagcaccaaagttggtggcttaggaaactgatacagttctttgaagactttcgtcagttgtctaccaagacccccaaacaggtggtttacttggaagtctcttacgaatctcacattgcccacatgtttggctacaaccacctccagagtctctctcttcagcatcccaggtcaggtgtgggtatcttgcattggcaatctcaaacatagaacctaccgaggagggaattctggataatatccctggcttctctgtcatgcagaggaaacctttgaagaagaaagatgcttatgtgaggacacacagtccggcatatttccctttcaccacttggttctacatatgccagaagcaacactgaggggccagctgccaaggagaaacatttctaagatatgtatattagctatgtctgtagctcccacagtaagtcaataagaaaaaggcacataatctgttagaaaaatggacagaggatatgaagagatcaaagaagaaatacaaatggccaacatgtagatactgttcggtccacttgtgattaaggaaatgcaagatcagaatatcttctatttaatttggaattattaaagtttatattagttttgaaatgtttagttttgaaaatataggagaaagctttctcctgtacctttgtacaaagaaataagagtggaacgtgtgtatgtggtttgcgtgggttgggaaatctacctactacatgattgatggccttgctcccaggaagtagtgatgtggatctgagtgagtatgggtgggagcattttaattttgtaattctaatttcaaaagagatttttattttattatttttttgagacggagtctcactctgttgcccaggctggagtgcagtggcatgatctcggcttactgccacctctgcctccagggttcaagtgatagaagagatttttttgaaaaaacagtgaactaattgtttactaggccccattttaattcagtcagtgttaagagttcaagcatcaagacaatctagtttatgttcaatgtaaattttctagttgctagaaatatttggatataatacagtcctttgctagaactgacattttaatggtgggaaaaactgtcagcctttagagatgccttttttcaagtttccgtgatactaaagttcccagctggtaacaaatgtatctgcttccttgagtttcaatgtccaggcctctttaaagtattcaggtatgcaaagacaggattatgcaagtttcttttagttggacaacctttattgatgaatttcttaggtttctgtggtagtgttgttagattaaatacagtcattccctcttgtctagtttcgttttatgtgatttcgctcttatggtttcacttaccccatggttcgaaagtattaaatgaaataaacaattcctgagttttaaattggacactgttttgagtagcatgatttgtccagtgtatccacactgtgggcactctccacacattagtcacttgttagccatctgggttatcagtattgcagtccttgtgttcaagtaacctttatttaatggctccaaagcacaagagtaatgatgtcgacaatttggatatgccaaaaagaagccttgaagtgcttcgtttaagtgaaaagatgaaagttcttgatttagtaagaaaaaaaatcaaatgctgagattgcaaagatctacagtaagacccaacctatctgtgaaactgtgcaggaggagcccattcattctctttttgccatcacacctcaaactgcaaaagttaggaccacagtgcatatattggaaaaaatacagggtttgttactatctacagtttcaggcatccactgggggtctcggaacatattccctgtgggtaaagggggactactgtatttttctaactgaactgtgatacgctgtaaccttttcttcatgacctccctcccctctacacagcttcttgctccccatagaaagctaggcagtgatgctagttaaaacacaaaacctattttagctgttagcctttcattgctagggttaattcccctgatagcagggccatttcagatcctagggtaatgtaaaattaaagttgtgtggcggatagtcacttggaacattttctaatgggagcttcaaacaatatggcatagtatgtacttttggccggattaatacagccacaagaaacctgtcttcagtacaggataatgattatttttcatgtcataacctgtgcaatctttgcttttactttgagtcagtaaatgagataccctatgctaatcatctagtgttcttattctggttttggggatatggtgtttacagagagtgtcaggggtgtctatatctgtttgtgtacctggaactgtgtcctcttgtccctgtattatctcagcacagcactgaccctgaaatggagtaccccaagcaatccaaagatgcaggacacctagaaattataagtactgcctgtgcatggcctccccttcctctcttgcacaatttgaaccacactttttctagtacaattctaggatcctttatcatgttgaaagctctccttcaggcttctttactgtgccacatttcttactaatatcctttctcacaatagtcttttcaaagcctagattcacacgatatcattctctagaccaaacctttcaatgactttatatggtacttgcaataaaattcataacctttactgttcactatctgtccttcacatctccaacctcagtgctctcttgatttcctccttaaaacctggccttattttttattcctaaaagtttcagcttaccttagaactgtggagatttcttcaaggaagaaatcggccaagcaggattctttgtaaaccctgcctatacctatttttatcccaagcacttcattattagtagtctgagtctctttgtaagtctctttgtgttccttagtaagcctttgatttatgtagtatgtcagttttccagccatggagtagtttaaacttgcacatccccctcttcttcagccccaaaggagaaactttaaaagtaaatccttcgccttaattaattatattatggaatatatattccttcctttaattaactatattatgaaataggggattcctattccagcctttctgggaactaagtaaggagtctcagactttgatagagatattatgtttcaaactgcatattctgcatttggctgtataaagctatccctttggaggaaaggttatctgagcagcaagtttaagttattagatttttatcaggaaatttggaagcatgaataaaaggattccaataattttttgtatgattattgatatagtgtattcttgtcatataattagatgaggaaattacataagaaacatgtagagtgaacattttaaatacatgcccatattaattgcagaacacaccctgatttcaaaaacattaaaatatcatttaaaaacaaagagggacatttaagcctaaaaattgacctggtaaagtggtttttagtaacagcaactttttgaggcttagcatgttttaatactaatgggatttttcatctgcttcttggagaaacctttttttaatgctaaggattaagttgaaagtagataagattaatacttagaagttataattagaagttaatgagttattaattagaagttaaactaatcattaaccagtgtttgttcatttcttcttacattgtcctattctcatctctagcactttgtcagcaacccctaagaggttagtaagtcttgaggaaaatgagtatgagttcaggaatatgtagatttgttcgtgtaagcgcttttcagttttagctttgtgttacctacttgctagcagaaatgaaccaaagactattttatggaaattaaagcatttcaacatatgcaatctttaggtgtttcttttttcctaagtcattgtgtaaatttcatgtataggaatagcaaaactgacatttgaagttgagtttacctatctcttatgtgtatagtcccttaccagaaaatactttttccttatgttgcccaggcaggatttgaacttctgggctcaagcagttctcctacctcagcatgcaccactgtgcttggctgttgtttttttttgttgtttgtttgtttgtttgtttaagagacggggtctcactttgttgccaggctgatctcaaactcttggactcaagtgatcctcccgcctgggcctcccaaagtgctaggattacaggtgtgagccacagagctcggccaaagaataaaagaatggctactccatgggcagagcagcctcttgatttttatgtatgttgatataagcaaattatctggaatttatctgctatactgataaaaatcagtaaaccttgttagtgtcagcatctaatctgtattaaacttttacttatttccctttactttttagattcaaagagagggttcacacagatatctttcatgctacattattgagcttaaggaagataaatttcccaaatatgatatttggtatatttgtgtgtctgtaattttttttttaatttaatgctgtatttaatttgtaagtcctgccattgactctaccagaggagattcttcaagcttagttgctgaacttcaagaaaagcttcaggaagaaaaagctaagtttctagaacaacttgaagagcaagaaaaaagaaagaatgaagaaatgcaaaatgttcgaacatctttgattgcggaacaacaggtctgtatctcttttcccatttcatttgagaactaaagaaggtagacataaattaacaaatataatttctttatgtagctgagggtttcttatatcttagatgtgatgttccttacattgatcctgttaataaatttcctcttgacaaaagcgtgtatttttaagaattttttttagtttttttactcatctggagaataatatttttcatgttaacatgtcacatgacaataccaggtgcttcaaaatgagatagtcttagattagaagcgagggaatgtaaattctgttcctggtttcttcgctacctatgtgatcttgggtagatcatttaacctcttgagaactaagattagttttctatgaaatgatgtttttaatttaaaaaatgattttattttttctctaagcttcaaattctctaaactttgttatcctaaattacattcagatcaacataaatatcttaccctttctgtaattcttatccttctgataggctggtggtagattaagatggaatattattgaattttgttaatgtacttactgttgttggtcaatagtaagaggtagtgcttgtgaagtatattggggttagataaaaaaccagatgccttgttagagagcttgtactttacctaaagtaccggacagcaactaattacaatggcataattagatttatgcttaatgaagatcacccactggaacatggagaaagattggagaactgcaaagcagtcagctggaagagcaatttggtagctactgctagaattcaggccagggataatattgcagtttggggtatacattttgagggatacatttctgaaactgcatatttatggttattgtacacatgtaatattatgtggtatttttccttttcctaaaatgttatgattaattagtgtctttgtggaatttgaaaaaatgtaaatcagagaacagaaagaaaataaagtatagttgaaacctctaacaattttagatttttaaggcctagggaaagaaagaagagcctgggaaaagggaatgagaaaagcacaaccagaaaaaaaagtgtgtggcttaagggaagccaaggaaagttaagtgttcagggaggaggaatagccaagttttaagtaagaattaacaatgaggaggtcagtagtaacctctctctaagaatctggatttatagattcttctctatgattcattctgcatattgatctctttatctccttgtgttgcattgtctctttgtgtgttctcttataagcacatgtacaatttctaaaattaagtaaagaatgaattaactgaaaccaaaagtatagtattgacagctgtctaatttacttcttcatgttttgatgtctcttttaatatttataaaataagagtgggagaaatgacttctaaggtaccttccaattttccatgatgtgtatgttaagttgtatttatttaaggttatttgtacctagctctgtataggtgatttctgagttggaactaattggttcttaactagcccccttttttcatatgccaattacaaattgtgatcttcctaaaaattcatttcaaattacagaaatggcaaaaatcatgaaaataaacataatggtttatatgtttttgtgtttattttctatatataaataaaatacatagagatggcttaatacaaagccatatcataaatgtttctttttgagagatcttcctgtatgctttgtggccagaaattgtatttaattaaatatttaatgaatttattaaaattttattaaaaatgctgtattacttttcctaaatctccctattgccagcattgttagcattattcattgatccaaggaccgtagagctcttaaaattttggaattagaaatgctgggcagatgagaagagtcagtttagatcatttcatcattcatctattgcttttacatttcaagaagctcaaaaatcttgtaagtaaatttttttttttttttttttttttgcctcttagagacagggtcttactctgtcattcaggctggaattcagtggtgcaatcatggcatgctataacctggaactcctgggctcaagcagtcctccctccttagcctcccgaatggctgggactacaggcatgtgccaccacacccagataatttttgtagagactcgatcttgtgttgctcaggctggtctcaaactcctgggctcaagcagtcctcccacctcagtctcccaaagtgctgagattacaggcatgagccaccaagtccggcctaagcaaaatttttatacagattcagtcatctttattaaagaaactgtgcagttaaaattgccagattgtctcatgagatccatgcaggcagttggatatgtgagcttcatagtagagtttataaaagtgggggagtaataagtctagccaatataaatcagcattccagtgaccattgcagatatccaaataagagaaataaggaagatagagtcatatctaaaatgttagggtggtgatcaaatgctaaagacaaaaggaaggtttcagaggcaggatgattactccaggaaaaaagtaagtgtctaacagcactctaagttattgatgcttctaattcttcaaaatttcaaaatctctgacagtaaatatatggacaataataagaaaataggatttgctttcaagagacttaaattggaaccaagaaagtagaacctttgccacagtatgttacattttcatttttaaaacatttcatgaatcataccccatcatctccatgtacaaatgtgccacagaaaagaatatttgcaagtgtattttctaagtcctagctgctaagtacatgtagataaatttaaataattattctcttgttccagaccaattttaacactgttttaacaagagagaaaatgagaaaagaaaacataataaatgatcttagtgataagttgaaaagtacaatgcagcaacaagaacgggataaaggtttgtactgttttgtactattttttttaattttttaaagtggaaaagatacattcatgcaaagtggtttttttaattacatgatacttttttaaatttgggtgtccttcagatttgatagagtcactttctgaagatcgagctcgtttgcttgaggaaaagaaaaagcttgaagaagaagtcagtaagttgcgtagtagcagttttgttccttcaccatatgtagctacagccccagaactttatggagcttgtgcacctgaactcccaggtgaatcagatagatccgctgtggaaacagcagatgaaggaagagtggattcagcaatggagacaagcatgatgtctgtacagtaagtatggctgttttcaactgtttttaaggctgttgcaattccatgagttataaagctctgaagagaggaaatatttttagcattactggttgtccacagcccatgtatctgaaagttgtgctattttaattctttaaactgtaagtgtgagaaaacattactgaatgaattattttggggtactctcatactatacaaagtatgtgcaccagaattatcccatactgccaagataaggatcatggcagtagtattgagccattcttatttacagggaatgttgacattttggggtccattacctataaaggggttattgtagtaatgaatatattcattttggtgaggaaaagcagggaagggttttttattattattattattatttgtttattcatatatagcaaatgtttactgcattacattgaaatcgtggcagagcttactggaatttagaactttcttaccagttctcctttgaatatagtgaaagaccactaaaattgtgcttacaattatatctatgggagagacagaattgggaggagaaagtgctcctctccaaaggacgttgcaagtagagtttgcatttctagttgcacctctgggatttcagatatgggaactgagattccaagggcagaaagtccagatcattatgccacacaaagaattatgcccagtgggtcagtgaacactacacccccatgtgagatgaagtaattcgaacaagtgattaccaaaagatatcatagaatcagcgtggtcccctcgttcaaacagatccaggttcaagtcctgagtctagcacctgcttgggaacttgagcaagtcatcatcccttatttttccatcaagataacagttatgataacaagacaacagttatgatacaccagcagggttgctttgggcattaatattttattttattttattttaattttttttttttttttttttgagatgaagtctcactcttatcccccagggtggagtgtgatggcacaatctcggctcactgcaacctccgcctcctgggttcaagtgattcttctgccttagcctcctgaatagccgggattacaggcgcctgccaccacgcctggctaatttttgtatttttagtagagacggggtttcaccatgttggacaggctggtctcgaactcctgacctcaggtgatccactcacctcagcctcccaaagtgctgggattacaggtgtgagtcatcgcgcccggccaggcattaaatttaatgagcacccgtgagaaggtgtggtacacagtaagaaaccagtaagtgcagggtccttcgtaccccacccctgcccactcagaggaacgcagttagttgcatcttcatataagcccattatctgcctgacctttgagaaactttggtaggggagttttttgtttgtttgtttttgaaaaataaaattacttaggtttctgtctggttgaaaggaaaataatagaaactggttacctttagactttgaaaggtataaggcttaagaatgtatattaaaaataaaatagaacaaaaaaagtaattcaaaaaagcaaatcaagaaaacatggttaagcaaatcaagaaaacatggttatttagcaaacatgaatgacataaatatgtccggacatgtgtgaaaaaagtgaaccaccgtatgaaaacctctttaaaaaaatatatccaacttcattctacatataggagaggtacctagagccaaatgatacagaaagtttactataatatgattcagaaagatatagaagggaaaaatataaaagcaattcaaagcaaaaggctttaaaaagctttaggaatttaaggcttttttgattgacacataaattatacatatttatggtttacagtgtgatgtagttacatgtgtacattcaaattagagtaatccatcacctcaaacgtttatcatttctttatggtgagaacattcaaaatgctcttttctagctattttgagatagcgcagtacattattgaacaccaaaacttattcctcctatctggctgtagctttgtacccgttgaccaacctctcctcatctcctctcctctgtactctccccagcctctggtaaccactattctgctctctgcctccatgagataaacttaagattctgcatattagtcagatcatatgtaggtttttttataaatatgaaaattgagttattcagaaccaaagtatcaaaaacagatatgacgaaagtggtactctgagagaactgtgtagttcctttatttttaaatgaattaataaaaacaaatgagccacaataaaataaaagttgtttagtagatattgggataaatagaaggtacaaataaatatatgtaatgaaaagagggacataaccttagttatagttttaaaaattacagagaacatgatacaagttaatctcacctgtgaattataaatacaaggaatcctgaagacaattttagcttatcaaatgccttgtggttttatgatgatatagtcatcatgacccagtggagtattttaggaatgccagagtaataactaaacagtacaatacctaaccatactcactgattacttgagaaggtttatagagtcatgtcagtagaagctgaaatgacaattgatataatgcagtgcccatttatttaataatttaacaaaatccttagcaaactaggagtagatgggaacttctttttatttgtaagtatatgggagagacatagcaagcatcgtatttcatattgaaacatttagaagtatttttcataaattcaggaagaacacaatgccagctgtcaccattgctattcagcaagacaataaataaaatttggaaacaactttttctgtcctcattttcagacagtatcctttacaaaccacaaagacctaaagacatattagaattaacagagtacatcagagtgtagatcatagatatatccaaagaggagtagtttgcatatttaccagcaaaagctgactttaatatgtataaaagatcccaataaaaatttaaaggaataacttacttgtaattaatttaacaaaaatgcgcaagacttcgtgaagaaaactaattttatgaagaggcctgaaagctgtacgtggaaagatagatggaaatggagtattattaagatatcatttttctcctcagatgtattcagtgatttaatctattactcaattaaaattctaacaatatgttaggttaaaaagtgatgtttgatgctagaattcatattgaaaaatgtgtaaaaataattgatagcatttctaaataaagatcagagagtagagatttgtcctgctaggtgttaagatatagtcttaaactacatagatagtatttaatgcagtacattattggtatagccacaggcaaatagattaatagagttagtaatcttgaaacaaaccagtggatatatatggaaatctacatgtagcattttaaatcagtttggtaaaaggatggactcctcagaatgggacattagaggcattgttgatctattggtgagaggtgagccatattttacattgtatatataaacaaaatgatagatgtagtaaagactaaatttttttttgaacttacagagtttcttaaagaaaataaaagactactctgaaatcagagtaggagagcacttggaaaacaataatcaatattacattgaaatttaaagcttgagtacaacagaaaacaaacagcagaagtctggtggttaatatttttaataaataaaagataaaaaccaaacttttaaataatgccagttttttataatctcttttaaaaaataggagagaactcttcccaacttatcctctgaggccagtattatatactaataccaaatcagacaaatatatcaaaagaagaaaatacatcttctataactattgacacaaatattatcggcaaaatactaatagatccagtctggcagaatacaaaaggaattccaattggctttatttcagaaatgcaaggttggtttaacatctaaaagtcaatttatgtcatacactatatcagtagaatgaaaggacagaaaaacacatgattatctcaatagatgaagaaaaagcatttgacaaaacccaacagcctttcatcataaaaacactcaacaaactaggaattgacttcctcaacctattgaaaggcatctgtgagaacccacaattaacatcatactcaatggtgtaagactgaatgcttttcccctaaaatcaagatgaagatggcctgctcttgccactgctgctcaacattgtacttggaagttctatcacgggcacttaggcaagaaaaagaaattaaaggcattggccaggcgcagtggctcatgcctgtaatcccagcactttgggaggccaaagtaggcagatcacctgaggtcaggagttcaagatcagcctggccaacatggtgaaacccagtctctactaaaaatagaaaaattagctgggcatggtggtgggcacctgtaattccagctacatgggaggctgagacaggagaattgattgaacctgggaggcagaggttgcagattgcgaccctgccctccatcctgggtgacagaacaagactctgtctcaaaaaataaatatataaataaataaataaataaataaatgcatctagattggtaaggaagaaaaaacactatttgcatacatagcctgatcttttattgagaaaatcctaaggtctcctctaaaaaaatactgtaattaacaaactagttcagtaatgttggaggatacaggttcagtgtgcaaaaatcaattggagctggctgtggtggtgcacagctgtggtcccagctactcaggaggctgaggcagcaggatcccttgagcctaggggtttgtgaacagggatgacataacaagaccctgtctcttaaaaaattgtcattctttctactagcaatgaatcatccaaaaatgagatttaaaagacagtttcttttctgatggcatcaaaaaggtaaaaatacttaggaatagatttaacaaaagaagttcaatacttgtacaaattacaaaacatggaaaaaagttaagacagtagggcatcctgtgtttactgactggaagacttaatcgccttaagatggtagccctccccaaattggtctgccagttcaacacaattcttatcgtaatctgaactgtgttatgttgcagaaaagggcaggctgatcctaaaattcatatggaattgcaagagtcccaaaatagccaaagcaatcttgaaaaataacaaagtctcacactgtttgactaatcttaaaatttgtaagcaagggtaataagacactgtagtgctagcctgactgtatcagtggaatagaattgagagtccaaaattagccctcgtattcatggtcggttgaacccttgcattttggttaaaggtgctgagacggttccgtgaaggaaatagctttttcagcaaatggtgcagggatagcatagctggatattctgatgcaaaaggatgaagttggaccctttcctcaccatatgcgcaggttgacttaaatagatcatagacttaagtgtaaaagctaaaattataaaactcttacagaagtcaatctttatgaccttatattaggcaaagcgtcttcctaaagaagtagcatgttgaagaagacagaatacctgccttgcatcaagttacttgaaatataatttggggattatacctgtagatgaattttaaaacctaaataataattgtttttctcttttctaattttatagcattttttaaaattttctaatttgtttttggatgttttagtgtattgggcaaattcacacctggcatagtaactagtgagtacatagcatccacttagtaaatctactgaagtctttagccactcaagaactacgactttatcatggattcactcacaaatattaagaagagtggttcttaatgcctgggaaaaataagaaatttaaagacatctctagataaatgcatacacagagttttacttattatttcaaaaggtttatagatctaggtctgctacagagaatatacaacattgaaattacatgctaactgaaataagctacttttgaaaatttatgaaaacccttaaattgttaaagtgatctgtattaatattttaatttctaaaatcttgaagaatgtaaagtttagaatttcaaactgaaactcaattattctcacttttagagaaaatattcatatgttgtctgaagaaaaacagcggataatgctgttagaacgagtaagtaaattatcttcttgaagtaccatgtttaatatgttcacttttggatacagttaaacttcattctggaaacatacaatagactatttgtgtttataaattaagcctttttatgattaacttatttttgtggtcactgttttgggaagtcttgtcattttatgatcttactagctaaacagttgacagtatttcagccctcccatttgttgtaatttcccaatgaatgaagtctgtatgagaggaaaaagttggtagtttttattaaatcaatgtgtaaagtaatagttttacctaatagttgagaattcagcaaaataacatgaatggcaattttaaacatctatttcaaaaacaaagtattaaatactttttagaaattggcaaaaacaaaaataagtaatgaagggagttatgttttaaataaagaattgaaatggacaagaaaaaactgataataaagggtttcctttattaagggtactataagatcaaattggtacacatttggcagatcattgttcatagattacttttcattctttaggcctataagattttcaacaatgcctttttttctttacaagtgtaattgtctgataaaaatccaaattcagaattgtacaacttcatttgaatatagcatgttttctattatgaaagggttatgtgatttcctacctcagcataacctgagatgtatgttacagatgccagatgtattctcttaggcacgacttagttaacaaagtctctggggatagatctcagatttttactacatatttatatattccacaggtgtttctaacgcatattaatgtttgacagtccttggtaaatgtatctaaaattgctctggttctcatttaagctagtagataatgaaaacatttggaaagaattcatagaaaataatatccccatcaacttttcatacccgaaccaaacatttttgtctgatgaaaactttgacaatcactgtttagatttatttgagtaggtacaaggataaaccacaggtctcttttttccacctgcagacattgcaattgaaagaagaagaaaataaacggttaaatcaaagactggtaagatttttcttcttggtaaattggtcttaacatttttaaatgatttgcattatatttcattatgaagatattccatcagaggatgtatgctttgattgacttatctatagtaggtttgttggtccgtgttggtcattgaatcctattgattttcagaatgctgaagtcaggccagagttatctcctgactgtagagtcagactttttccttagcgtgaaattaaataatgtcttggcgaatttacggctgtaggtctgaccagttgaagcacctaagattcttaaaatcatcaaaatcagttttgagttatgtttcataagtcttttgaagattaatatatttactcacaagtgtgcgtaattttaatacattgtatcttttcatgattttttttttttaagacagtcttgctctgtagcttaggctggagtggtgtgatctcgactcactgcaacctccacctcccgggttcaagtgattcttgtgcattagcctcccgagtagctgggattacaagtgtgctgcgccgtcacacccggctaatttttacataattaatagagacaggatttctccattttggacaggctggtctcaaacccctggcctcaggtgatctgcgcaccttggactcccaaagtgctgggagttacaggtgtgaaacattgcacccagcccttttcatgaatcttatttttaagtgaaagtgcctgaaaggcaagagatcccatgtggtactgtaaattgccctggtttttattgatttgttttgtttcgtttttaattaaaaaaaaaaaatcgagacaaggtctctctctgttgcccaggctgaagtgtggtgttgcgatcttggctcactgcagcttcaacctctttggctcaagcaatcctcctgtctcagctcagcaagtagctgggagtacaggcgagtgccgctgcacccagctaatttttgttttgttttggttttaggttttgtttttttgttttcgtagagatggggtttcaccatcttgtccaggctggtctcgaactgctgagcttaagcgatccgcctgcctcagcctccccaagtgctgggattccaggcaaattggaattgcctggaattttgctgcacccaaccaaactgccttggctttaacctgtgaatttaaccaattatttgaacagcccattttacagccaattatatgttttaataaagcaataactttattttttcaagttctattaattacagtttacaaggtagtaggaacagtttaaggcttctgttcagtgcttttgaaaagttttgaaatactttttagaatttagtttgtttaaaaaataaaaatatcttgtagtgagacatctcctttcttctttataccttaacttgaacagcaaaaatcacatattttgtagtgattatactgtgaatcattaattttatcacaaagacttagaagaatggtctatctcagacatgactcaatacgtgttttatgtagtatgctttatttatcctcaatagaatataagctctgtttggcaacgattttaatcagttttggtcaccgctacattcttagcatctagaatggtaactggtatactatagatgcttggcatatgtttgtagattaattgtgaacccaaaagtatctgagacaggtctcaatcaatttagaaagtttattttgccaaggttaaggacgagctcatgacacagcctcgggaggtcctgacgtgtgcccaaggtggtcaggacacagcttggttttatacattttagggaaacatgagatatcaaccagtatgtgtaagatgttctgtctggaaaggtgggacaactcaaagctgggaagagggcttccaggtcataggtagataagagacaaatggttgcattattttgaaactgtgattagcctttcactgaatacacaatttacatgtcagaggatggtagaggaattgtcacttatgcttcagtctgacttagtgaagcagtggggccgagaaagcaatcatatacgcatttgtctcacatgagcagaggaacagagggatgactttaagttctgtctgttttttgtccacaaggaattttcttgtgggcaaattgtgaggtctttgtagctatcttattttaggaataaaatgggaggcaggtttgcttgatgtagttcccagcttgacctcccttttccttagtgatttttggttcccaagatttattttcttttcacagaataaattgtctttcagacccagagagcatcacagtcacattcagaaaggtgtccaaatgtaaatcacactttcacatagaattacagctatattaacaaattttttcttccattgtcttcatttgtaatatataaaaaacttaagcttttaaaaaactaaagttgaattatggtcttaaaaatgatggtcaatcttatcttcactggcaggatatagaccatttgtctggataattttagtaagttgctatacagttttaggccttcctagttattatttgttgtgttatttctctttttaaaaccctgcattgtaccagatagcgttcagagggaagtggaggaaattcagagcatcaaaatttgacttaataaacaaaatatccttatctgtccttggtttgtgcaaccatgcattgggctcactttgcttgggaaaaataggtgtttttatcaccctttcaagcttcaaagaccttggtttccatgcttcaggtcttatgagatggtgtggataaatggattgaagcttgtttgaagaggagaacagtcccggctaagccatggctatagcttatcagctagcaagtaccatttgattgtgctcatcatcagtagaggaagtgggtggctatcacaaaattcattgtttcgtgattgttcacaaatttttttaaactgagacatctcagaagtttcattgaagatgagaaaagaggtttgttctcagccttcagaactttatgctcccaccccagtttaaccagagtagtagtgtttgctaggcaaggtgtcagagcgctggcattgcaaagtggttgactctggtttggtaagaagaatttactgacaacagtataggtttgaaaaaggaaagttttattagagcgaatgatgctgaagagtgtagcggggtgcctcagcaagagaggactgagcacgctgtggtgaattttcccgacgagcatatatggaccttaaggcaggatcttaaaggtagtttggaccaatgttggccgtgtaggtcatgataaatgattacatttgtagacatttttgtgtcttgatgtcagcaagggttgctcagtgagttttggcatgcatacattccagagatgtatagaaattctagttacttacaacatttttgaaagagacctgtaaccacatgctaggaaagtttaattacttctaaattcctcagcgttctgcctccagatggcctgtttgatggtcaccaggtgtctttgcttgctgcattatacttctaacctctgccttccaggctcaagtgatcttcccacctcagacttccgagtagctgggaccataggcgcttgccattatgcccagctaattttttgtatttttggtagagatggggtttcaccattttgcccaggctggagattttaaatctttttggtttttttgttgttgttcgttgggacaactaagttcctcttcaaagactcaacttcctggtcataagttgtaagattgtaaatcaaccccatccccttctcccctctgccttctccttttctcacaaatcgcacatttaccctatttggaaaaagtttaggtctaagctaaccgggatcagcttagattgtgtggtccaaccccagccaataggggaaggacacagaaacagaaactgcattagagataaaaccccttccttcctttgtttggtgttctcttgcaattgtgacggctgtgagcagcagccttctgcagaagtaaagttgccttgccaagaaattttctaagtgtgggtttcttttggctacactgagcacttgtttccaacattgttgtttttccaaaatgaaagattttgaatctaatgcattagaattttcaaagtcttcttgaaaaatctgtccttttctctgtgttttatttttaagattaaatatgtaatataaataaaatgtgattcaaaaatcagtttttaatctaaccaaatttactaatggttgaaataaaggaattattaatgaacataagaactatattagtttgggctctttaaagctggacacaggcttgaatgtgtataacagtgggtgaaatttttgcagtaagaatgcatgtggtactcaaattgtgtaagttcataaactttttttttttttttagatgtctcagagcatgtcttcagtatcttcaaggcattctgaaaagatagctattagagagtaagtattttagttttgtgagtatttttctgtgaaaaacattcactgaattgttaatgaaaatgtatttcagtgacagtaattcaggtccaggttcattgagctgagtgttttgcactcagtagtagtaaaagttttttactggaaagcaggatttttctaaattgttattctggaaacatttagattgccaaaactttccagttatagcattatgattaacataaatatccttgaagccttttcctccagttaacttttcagcatgtgctacctcttttaaatcccatttatctccttagtcttgttaatctatattggagagttttgatttgtttttaagttgcagtgtttcctcatttttagtttttagatttcattttattaatattctacaaattcataataaacaaattgtcaatgaaaaagtcaaactctgtaaaatatttgaagatatttattctgagccaaatatgagtgaccatggcctgtgacacagccctcaggagcccctgagaacatgtgcccaaggtgattggatgacggtgagggggaaacttggtttcatacattttagggagacatgagacattgaagatacaagttggtttggtccagaaaggtgggacaaatgaaagctgggagggtagcttccaaagtataggtagatttttaaagttttctgattggcagttggttgaaagagctgtcagtagaaaggaatgtctaggttaagataagaggttgtggagaccaaagttttgtcatgcagatgaagcctccaggtagcaggcttgacagataatagtttgtaaatgtttcttatcaggcttaacctctgtgttagtgttaaattcttgttgacttttcctgaattctaagagggaagaggacatgatgaggcaagtccaacccctgccttttgtcatggcctgaaccggtctttcaggttaaatttatagggcactggcctagaggaggaagttcattcagatggttggttgggggggcttcaaattttatttttggtttacagaatttatcaattgaatggtttaataattttcctgcactgtattaccccatagttaaaatttttctttagctttggttatttaaatggagctctggaataataaaagcattgttagcaacttatgaatagtgttcagccttttatcctgacaggaaggttgtgaaagttaagaccctttatttaacttcttgtattttatttctaaatttctaaatttgttgaccacatttttggttgtcagatatctgtgattctgacttttcatgtctcattaaaatttcagcttctctcagtgatttgaaagggcttagtggtatctgtctttaccagctgtatgtcatgtcaatgggccgttcattaatgttctttcagagactagagagagaggagctccaaaaatactgcctcatagaggccccatttcccatctccccattttaagttattagcttttggcatttagccaaaacagtgcctgccacatggttgttaggtaatcactaagtatttgtagggtaacttaataaataaaagacattaaaagtttccccagctgattttgatgatttattaggtataggatctaccaatttaaatgatcttgacaactgaaaaatatgagattgattttcaaggccttttgatgttttgaacaacagttctgaaaaaggctgtaaagagacaaacaccacttaagcatgtaattggttgtacccacttctctctttccctgtcttctttcgggcttataccccaaaatagtaatatctgttgacttgtttagacctaattaagcacctgacttctggacctagctcagttgctttttcttgtaaattgaagagcactacgcttggagacaagatctaatttttctttgacactgtgactgtatatgagatgtctctctgaatttttggtgtgaaatgaaagattaggagaaaaaaatcttgctatgactggataaactgtaatgggtgctttttctgcttttcatccagttccttaacgttagataatggaaactttattaaagaaatttttggtttttgacagcccctgacatcatcctttgaatatctgtgcatgaccagcagtcccattaggacctgggcagaatgcaaaaagagtatcatgtaaatgtgaaatttaatattgcattaatatcttaatatgtagttgagcattaagtatatatgctaagttttcagaatagggggattgaaagtaactctaagtcaataaaattgcatttctttattcagttttcaggtgggagatttggtactcatcatcctagacgaacgccatgacaattatgtgttatttactgttagtcctactttatattttctacattcagagtctctacctgccctggatctcaaaccaggtgagggtggtaagtgtcattccatgactaaaatataaacctggaatggaggaaattatataagtaaagatttttttttccctgtttaattacataagaattaagtcactaagagggaaccctgtgtaatctgcttttttataaaatctgtattatctagattattttcatggattccatttacaattcagaattaaggagtgtagcttatacaagggaccagttgatcagaatgtgcagtgattcggacatctcataaccctagcagttggataattatattgtcaaagttctcatgtacagactattcaaaattaatgtatgcttatttgtttatataaaagcattattttttggcaaaaaaaactttcttaaattataaaatgtctagtctatcctatatcatcctatgttcctaccttgtttatcattatactgttctatacacttatttttttaaaaaggaaataagctttgttaaaatatactaagtaacaatgtctattatctatttgatttttaaaatactaaaaaagaacagaatagatatcctggtgtacttagcctaagtagaattatttttaacactaaaacgaagtaagtccatttagattgatgttcctaatctaattggactgactcgcaacaaagtcaagtatcttatctatatatttataggtagatattttttttcgaaacggagtcttgctctgtcgcctaggctggagtgcggtggcacgatctcagctcactgcaacctctgcctcccaggttcaagcaattctcctgcctcagcttcccgagtagctggaattacaggcgtgtgccaccacgcccagctaatttttgtatttttagtagaggcgggtttttacctgttggctaggctggtctcaaaactcctgaccttgtgatctgcctgcctcaggctcccaaagtgctgggattacaggcttaagccaccgtacccggccttttttttctttaagacaagtttttgctctgttacccaggctggagtgcagtggcatgatcattggtcactgtagccttgaactcctgggcccaagcaatcctcctgcctcagcctcctgagtagctgggactaggtactatagatttgcaccaccactatgcccattttttgttttgttttgtagaggcagggtctcactttgttggccaggctggcaaattcctagtcttgaactaggccactttccccatgacaactccatcatggctatttgtggttggctgtttagagtatccatgaaacagtaatcatgtaaccatgaaaaatgagaagatagcttataacttactatttttacttagtactttttaccttgaaaataatgaacctgatatttcttttgtcttttatggggttttttgtttttcagttgtttaaaccttttatttgaatatgatttttaaacttagaaaacgtcgcaaagattaatatagtacaaagaaaacctatatttctttacccagattcatctgttaacattttaccccacttgcaatatctttttatgtgtatgtatctaagatataaatatacacaaatgtgtgcacacttttttcccccagaaccgtatgatgataagttacatcttagcccattacctttcaacatttccatgttcattttgaagacttcagtgtttaaatctgggaaacattaaaaaacttttttaaaaatgcacactgccattttaaaggctttcacaagtactgcagaaaagaatttatttatttagcagtcttacttaaccattaataaacccccgttccttttttcctttgaggaatgccagttgggatcatgctacccctgtgttgttgccttgggagagtgcggattaccagagtgtttttatgccttttgttagtggggctcattactcttctggcatcatacaagcatttatttttagatgattctttttggatgttagtaattggctgtgaagtgctagtttgctcttcagagaattcaccacatcctattgtccaggggcttttctgtttctgtcatgtgatcgtattcttcaggttcatttctgattatttatttgggatatctttcgtgtatatcttcccaaattatttgtatggaataaaaatccgtatgccttctttcaaaggctttagcctaaacacaatacagacagttcccaacttatggtagttctacttaaaactttttgactttaccatggtgtgaaagtgatacacattcatagaaaccctactttcggtatccacacaaccattctgtttttcacttttagtattcaataaattacatgagacattcaacactttaaaatatgctttttgttagatgattttgcccaactgaaggctaatgtaaatgttctgagactaggctaagctatggtgtatggtagattagaagtattaaatgcattttcaacttagatattttcaatataggatgggtttaatctggacataaccttatcataagttgaaaggcatctgtagtttattgcctgaatacttttgaagtcaactttaataagacagcttcttttctgatgaatttaaaaagagaatgggtaccacttggcttgtcacattttggctttatgaggactaggattatcctgtcttctctcacatttttcagctttccttcattcacacaagtactgcccttctagaattttttggttcttagcccttattaatttattgagtaccaactgtatacacactgctgtgccaagcactgtggcatacagatgtgagaactgttcacttgctgcctttgagaacttgtattaaaaatactgcccatcaaaaatgagtgttttgctttgaacctatacattttgtttgcttaaagaaatgggaaaggtcatctttattagttctttcaatagatggcactactgagttagcctagaattcctacatatttgttcatgatttgtatttagattattagtgtttcctgaactgttatttctgtgatttgacatatcatgtatatttaatgagtaacaagagctacgcacacattactggttgaaaagtaacatgaatttagttttgtaaattgttaagcatttcagtgatttcggtagtttttaaagtcttacatattcttccactcatgagagcttttttagtctgttttaattctggcagttatgtttccatgaataattataatcatactttcaaaaagagattaactatttaatttcattagcttcaggtgcatctagaagaccctgggtacttggaaaagtaatggaaaaagaatactgtcaagccaaaaaggtaaaaacgacatcatgtgatactaagcctgggaactgtaaagaagattttaatactgctttatatagaaatcattaccactgctgttcttggcctttatgctcatttcagttagagcttgaatttaatgagagtgagtttgaactggcaatttataagtattcatgctgcccttagatagtagagatagattaactacaagctatcaacatataatggaaaactactacgaagtctgtgttctatgactgttgggttgctagtgtaactaatttgcaagaaaaaaaatccctgcataactagagacaaaaagggaattttatttataaatacagttgatcttttattattttcatactctgcccccattgaagaaaggtagtgtaaagtatttggtggcttaacaaatggactgattgtataacaatatcagtgctctagagtcagtgaagtcttctgagcattcttcctgactcactataatttggggcctcaatgtacaaaatacgaatagaacttgaaccatatagtattgaggtttctttgatacttaaaacaatgatgtatggcccactttgtattatattctttagcagtggctaccaactttgtctttcctattcactcaatatattccctgttcgtgaggggtagatcttacatagtttgataaagacccccacatccctccaacccctcaatactcagcaatgagtaatatgataggagggtacttgtgtgatgtggtttatttatttttttgtttctttttgttttttgggtgtgtgtgtgtgtgtgtgagagagagagatgtggtttaatcactctagtgattccattttttctttccctcttaggcacaaaacagatttaaagttcctttggggacaaagttttacagagtgaaagccgtatcatggaataagaaagtataacttatggacaaaattaatacattctatgacatttttttctgatttgtcctgcagtgctcattcatcactccaaaaacagcaggccatctttttatgcaaaagtcagcgtgacaatatacttcactggtgtacatcgtttactttttaactggcttcattttaggaataataaattcatcagaatccttggctgaattaaaatggtttttgttttttggtttttttttttacccagacaactctagaaatgcggaccaaactacttcattttctcaaagggcataccttgtgcattgtggcttatgatgagccatattaattgcctgttaaatatacactagcttgaacttagatgttaaatgttattattaccagcatttgtccttttgtgaaatcagtatcagaatacttgcactctttaacacattctttataaaatgtataaattattcagaactatttaaaataaagaggagtgttattgcatgctgataatcattttgagtttgcctcagtagatactaaagcaaattgtttcagtttttttaaatgccctttgatgtttcaaaaaaaaaaaggaactgtaatttgattgactgattttaagatcagccataagtaatcagcaatcttcaaaagcactttcagtggattggtcatctgggttctaaagggaagagtctgtgctactaaccatttcaaatgcagactcaaaccttcccaacatctttatgactctagaataatcatattgatgaaatcgtaattcatggttgagtttcagaacaaaagatattcattgcacattaaccatttagaggtcatttaaataacaaaatattgtattgtaaaagaactgtacaattttaaaacaataaagatttgaacctgtaaatgtgtgtgccttttaaagaaggatacatttttaatatatttgagtgattgctgggaagtgtgaaaatattgttatgtatcatatcaaagagaaacatgtttattacaaaaatgttctttaactatatactatgtaacagggtaaacagtgttatgtagaatagaattgtgtaaactagatctttagagaagttgccattgagcaaagttatttaaatgagttagttgagttggatgagaattgtttgaggtttgttgctagagaacaataataaaataattctttttcagaaaatatttaatttcttcataaaaataagttaaatatttttttaaatatgtatatctaatagtacaaaatggaataaacatcatagtgtatagaaaactgaatttgacaagttaatgaataaatgaacaaatgatttca

SEQ ID NO: 19 - protein, 1594 aa, Homo sapiens
MKLYVFLVNTGTTLTFDTELTVQTVADLKHAIQSKYKIAIQHQVLVVNGGECMAADRRVCTYSAGTDTNPIFLFNKEMILCDRPPAIPKTTFSTENDMEIKVEESLMMPAVFHTVASRTQLALEMYEVAKKLCSFCEGLVHDEHLQHQGWAAIMANLEDCSNSYQKLLFKFESIYSNYLQSIEDIKLKLTHLGTAVSVMAKIPLLECLTRHSYRECLGRLDSLPEHEDSEKAEMKRSTELVLSPDMPRTTNESLLTSFPKSVEHVSPDTADAESGKEIRESCQSTVHQQDETTIDTKDGDLPFFNVSLLDWINVQDRPNDVESLVRKCFDSMSRLDPRIIRPFIAECRQTIAKLDNQNMKAIKGLEDRLYALDQMIASCGRLVNEQKELAQGFLANQKRAENLKDASVLPDLCLSHANQLMIMLQNHRKLLDIKQKCTTAKQELANNLHVRLKWCCFVMLHADQDGEKLQALLRLVIELLERVKIVEALSTVPQMYCLAVVEVVRRKMFIKHYREWAGALVKDGKRLYEAEKSKRESFGKLFRKSFLRNRLFRGLDSWPPSFCTQKPRKFDCELPDISLKDLQFLQSFCPSEVQPFLRVPLLCDFEPLHQHVLALHNLVKAAQSLDEMSQTITDLLSEQKASVSQTSPQSASSPRMESTAGITTTTSPRTPPPLTVQDPLCPAVCPLEELSPDSIDAHTFDFETIPHPNIEQTIHQVSLDLDSLAESPESDFMSAVNEFVIEENLSSPNPISDPQSPEMMVESLYSSVINAIDSRRMQDTNVCGKEDFGDHTSLNVQLERCRVVAQDSHFSIQTIKEDLCHFRTFVQKEQCDFSNSLKCTAVEIRNIIEKVKCSLEITLKEKHQKELLSLKNEYEGKLDGLIKETEENENKIKKLKGELVCLEEVLQNKDNEFALVKHEKEAVICLQNEKDQKLLEMENIMHSQNCEIKELKQSREIVLEDLKKLHVENDEKLQLLRAELQSLEQSHLKELEDTLQVRHIQEFEKVMTDHRVSLEELKKENQQIINQIQESHAEIIQEKEKQLQELKLKVSDLSDTRCKLEVELALKEAETDEIKILLEESRAQQKETLKSLLEQETENLRTEISKLNQKIQDNNENYQVGLAELRTLMTIEKDQCISELISRHEEESNILKAELNKVTSLHNQAFEIEKNLKEQIIELQSKLDSELSALERQKDEKITQQEEKYEAIIQNLEKDRQKLVSSQEQDREQLIQKLNCEKDEAIQTALKEFKLEREVVEKELLEKVKHLENQIAKSPAIDSTRGDSSSLVAELQEKLQEEKAKFLEQLEEQEKRKNEEMQNVRTSLIAEQQTNFNTVLTREKMRKENIINDLSDKLKSTMQQQERDKDLIESLSEDRARLLEEKKKLEEEVSKLRSSSFVPSPYVATAPELYGACAPELPGESDRSAVETADEGRVDSAMETSMMSVQENIHMLSEEKQRIMLLERTLQLKEEENKRLNQRLMSQSMSSVSSRHSEKIAIRDFQVGDLVLIILDERHDNYVLFTVSPTLYFLHSESLPALDLKPGEGASGASRRPWVLGKVMEKEYCQAKKAQNRFKVPLGTKFYRVKAVSWNKKV